(19) United States
(12) Patent Application Publication
(10) Pub. No.: US 2004/0175434 A1
(43) Pub. Date: Sep. 9, 2004

(54) Antimicrobial compositions

(75) Inventors: Charles S. Schasteen, St. Louis, MO (US); Jennifer Wu, St. Charles, MO (US); Pierre Buttin, Nantes (FR); Pieter Hillebrand, Breda (NL); Fredrick R. Scott, St. Louis, MO (US); Mercedes Vasquez-Anon, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Novus International, Inc.

(21) Appl. No.: 10/652,745

(22) Filed: Aug. 29, 2003

Related U.S. Application Data
(60) Provisional application No. 60/407,050, filed on Aug. 30, 2002.
(60) Provisional application No. 60/441,384, filed on Jan. 21, 2003.
(60) Provisional application No. 60/441,584, filed on Jan. 21, 2003.
(60) Provisional application No. 60/456,673, filed on Mar. 21, 2003.
(60) Provisional application No. 60/456,732, filed on Mar. 21, 2003.
(60) Provisional application No. 60/465,549, filed on Apr. 25, 2003.

(51) Int. Cl. A61K 33/22; A61K 31/195; A61K 31/19

(52) U.S. Cl. 424/659; 514/557; 514/562

(57) ABSTRACT

Antimicrobial compositions and combinations comprising a compound of Formula I

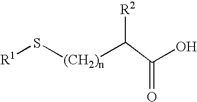

wherein R¹, R², R³, and n are as defined herein, are disclosed.

### REFERENCE TO RELATED APPLICATIONS

This application claims priority from provisional application Serial Nos. 60/407,050, filed Aug. 30, 2002; 60/441,384, filed Jan. 21, 2003; 60/441,584, filed Jan. 21, 2003; 60/456,673, filed Mar. 21, 2003; 60/456,732, filed Mar. 21, 2003; and 60/465,549, filed Apr. 25, 2003; which are all hereby incorporated by reference in their entirety.

### FIELD OF THE INVENTION

The present invention generally relates to the provision of an antimicrobial composition and of methods of controlling the growth of, preventing the growth of, or killing, microbial organisms. The present invention also generally relates to methods for inhibiting or killing microbes in water and food, including human food, livestock food, pet food, or other animal food; methods for inhibiting or killing mold in water and food; methods for inhibiting or killing bacteria in water and food; methods for inhibiting bacteria in water and food; methods for inhibiting mold in water and food; methods for killing bacteria in water and food; methods for killing mold in water and food; methods for delaying formation of mold in water and food; methods for enhancing digestibility and/or palatability of water and food; and methods of delaying or stopping mold growth in water and food.

### BACKGROUND OF THE INVENTION

Control of bacteria in animal feed is an ongoing challenge for the industry, even more so as consumers focus on the health and safety of the meat they feed their families. Control of bacteria is desirable to protect the health of the animal for which the feed is intended. Gut bacteria in the animal compete for nutrients and can be detrimental to the health and performance of the animal. Second, and of growing importance, the reduction of bacteria in feed is an important part in the reduction and control of food borne diseases.

The use of antibiotics in animal feed has recently come under scrutiny, in part due to concerns that overuse of antibiotics may result in resistance to the antibiotics in the animals treated. Additionally, upon consumption of animals treated with antibiotics, humans may experience problems due to allergies to the antibiotics, or in becoming resistant to the antibiotics as well. Thus, a need exists for alternative means of inhibiting bacteria in animal feed.

Two main alternatives are currently available for control of bacteria in feed: thermal and chemical methods. Heat treatment of feed is costly, but efficient. However, heat treatment alone does not avoid re-contamination of the feed between the time the feed is treated and consumed by the animal.

Chemical treatments primarily involve the use of formaldehyde or an organic acid to the feed. Formaldehyde has been used extensively in the UK, although its use remains under scrutiny by the EU Commission.

Organic acids, on the other hand, have many applications in the animal feed industry worldwide. For many years, nutritionists have used organic acids in piglet diets for their positive effect on health and growth. For instance, formic acid is used to decontaminate raw materials, and propionic acid is used to control mold. Other organic acids commonly used include fumaric, citric and lactic acids. Organic acids are also known to modify the gastrointestinal flora.

Approvals for the use of non-therapeutic antibiotics in animal feed have been withdrawn in several countries and the practice of feeding low-dose growth promoters is fast disappearing worldwide. Already, producers who want to sell in the drug-free market are searching for non-pharmaceutical replacements for feed antibiotics. Most data suggest that the growth-promoting effect of antibiotics can be entirely ascribed to their antimicrobial activity and the physiological repercussions from that. Thus, the search for replacements has focused on naturally occurring molecules with known anti-microbial activity.

The primary effect of antibiotics is antimicrobial; all of the digestibility and performance effects can by explained by their impact on the gastrointestinal microflora and the resulting reduction in immune stimulation. Organic acids have antimicrobial activity; however, there appear to be effects of organic acids beyond those attributed to antimicrobial activity. Reductions in certain species of bacteria are associated with feeding organic acids, which are particularly effective against acid intolerant species like E. coli, Salmonella and Campylobacter. Both antibiotics and organic acids improve protein and energy digestibility by lowering the incidence of background immune stimulation and the resulting synthesis and secretion of immune mediators, by reducing production of ammonia and other growth-depressing microbial metabolites and perhaps by reducing the overall microbial load. Unlike antibiotics, the antimicrobial activity of organic acids is pH dependent. Organic acids have a clear and significant benefit in weanling piglets, and have been observed to benefit poultry performance. Organic acids have several additional effects that go beyond those of antibiotics. These include reduction in digesta pH and increased pancreatic secretion.

Pigs are susceptible to weaning stresses (separation from sows, environmental changes, and physical effects of solid feed) and a variety of pathogens such as E. coli and rotavirus. These pathogens are reduced in adult animals by the reduction of pH in the stomach, but young pigs have lower hydrochloric acid secretion from the stomach. In addition, the failure to acidify gastric contents coupled with low pancreatic enzyme secretion can lead to insufficient nutrient digestion and also increase the susceptibility of weaning pigs to enteric diseases.

A number of studies have documented the effects of organic acids on performance in young swine, particularly early weaned piglets. A recent publication by Partanen (“Organic acids: their efficacy and modes of action in pigs,” in Gut Environment of Pigs, p. 201, Piva, A. et al., eds, Nottingham University Press, Nottingham, UK (2001)) reviews the literature in this area and provides the results of a meta-analysis of existing data. Only studies using individual acids in the absence of antibiotics and copper are considered. In the analysis of 46 weaned piglet and 23 fattening pig trials, significant feed-to-gain improvements were seen with formic, fumaric, and citric acids and also with calcium diformate. Weight gain and feed intake effects were significant for formic acid and diformate. The author concludes that dietary acids have a beneficial effect, especially on weaned piglets, that is primarily associated with changes in the gastrointestinal microflora. See Eidelsburger et al., J. Anim. Physiol. Anim. Nutr. 68: 82-92 (1992).

Without being limited to a particular theory, it is believed that one mechanism for the action of organic acids as antimicrobials is as follows. The acid changes the microbial populations in accordance with its antimicrobial spectrum of activity. Once inside the cell, the change in pH causes the dissociation of these weak acids. The multiple effects of organic acids are due to this intracellular dissociation and the cellular response to it. The antimicrobial activity is a result of the deleterious effects of the free proton and, perhaps, the free anion on the bacterial or fungal cell. In the gut enterocytes, the dissociation is thought to result in the synthesis of secretin, a hormone that stimulates pancreatic secretion. Thus, organic acids have benefits that go beyond antimicrobial activity. For feeds, the activity to control fungal growth dominates, while in the gut, the populations being affected are primarily the bacteria whose growth is most affected by acidic conditions. It should be emphasized, however, that the mechanism of action of organic acids is quite different from, and in addition to, that of inorganic acids such as HCl. See, e.g., Eidelsburger et al., supra. The importance of low pH on the antimicrobial activity of organic acids can be explained by its effect on the dissociation of the acid. At low pH, more of the organic acid will be in the undissociated form. Undissociated organic acids are lipophilic and can diffuse across cell membranes, including those of bacteria and molds. See Huyghebaert, Report: CLO-DVV (1999)and Eidelsburger et al., supra. Once in the bacterial cell, the higher pH of its cytoplasm causes dissociation of the acid, and the resulting reduction in pH of the cell contents will have the effects of disruption of enzymatic reactions and nutrient transport systems. See Cherrington et al., Adv. Microb. Physio., 32:87-108 (1991). In addition, the process of transporting the free proton out of the cell is energy requiring and this contributes to reduced energy availability for proliferation, resulting in some degree of bacteriostasis. This direct antimicrobial activity is believed to be responsible for feed and food sanitation effects that contribute to the use of organic acids as preservatives.

Most food production animals, especially pigs, poultry and cattle, require supplemental methionine in their diets for proper growth and reproduction. 2-hydroxy-4-(methylthio)butanoic acid (HMBA, sold under the trade name Alimet® by Novus International, Inc. (St. Louis, Missouri)) is a popular source of supplemental methionine for animal diets. Alimet® has become a preferred source over powder d,1-methionine (DLM), for feed mills wanting to solve the common problems associated with increased production capacity because of the efficiency advantages of the physical form of liquids. Easier bulk handling, accurate dosage, elimination of packaging with its disposal and the inventory shrink issues, as well as dust reduction, are popular features. Alimet® contains 88% methionine activity, while liquid DLM contains only 40% methionine activity. This low level of relative activity in DLM means water takes the place of valuable energy and protein components in the feed, reducing nutrient density.

Preventing or delaying the growth of mold in animal feed compositions is beneficial, in that less feed is lost to spoilage, and illnesses associated with the molds or toxins they produce can be avoided.

Mold in feed rations can render the feed unfit for consumption. Moldy feed may decrease the digestibility and/or palatability of the feed, both of which can adversely affect production and health of the animal. Additionally, many molds produce mycotoxins which affect the nutrient value of feed, or which may be hazardous to the health of animals, including livestock and humans. Aspergillus aflatoxin B₁ mycotoxin is a potent liver carcinogen; certain Penicillium mycotoxins affect liver or kidney function; and Fusarium mold species are associated with pulmonary edema in swine, liver cancer in rats, and abnormal bone development in chicks and pigs. United States Department of Agriculture, “Grain Fungal Diseases & Mycotoxin Reference,” available at http://www.usda.gov/gipsa/pubs/pubs.htm. Pigs are particularly sensitive to the presence of Fusarium mycotoxins, especially deoxynivalenol (DON), also known as vomitoxin.

Detoxifying feed which has been contaminated with mycotoxins can be quite difficult, and often is accomplished only by subjecting the feed to extreme processing conditions. For example, corn contaminated with aflatoxin, a mycotoxin produced by the Aspergillus species of mold, can be detoxified by treating the corn under pressure with hot, moist ammonia. Thus, the need exists for a way to prevent formation of mold and the mycotoxins they may produce.

Mold growth in untreated, stored feeds is especially prevalent in hot, humid conditions. Higher temperatures increase the chance for mold growth, particularly when coupled with high moisture levels in the feed. In chicken feed, mold growth rarely occurs in grains containing less than 14-15% moisture. However, even feeds with a low average moisture level may have pockets with high moisture levels, producing microclimates ideal for mold growth. This often happens in warmer climates, especially where cool night time temperatures cause condensation inside silos or bins where grains or feed is stored. Even these relatively small pockets of mold growth can be problematic; mycotoxins can be problematic to birds at parts-per-million levels (e.g., tricothecenes, produced by the Fusarium species, and aflatoxins, produced by the Aspergillus species).

It is known that certain feed additives prevent mold growth (i.e., act as antifungals) or bind existing mycotoxins. Such additives are often used where moisture control in grain or mixed feed is logistically difficult. The use of certain organic acids, including propionic and acetic acid, as mold inhibitors is known. Adsorbents to bind mycotoxins include aluminosilicates and clay loam products, whose chemical structure allow the capture of aflatoxin, and perhaps other mycotoxins. Mycotoxin adsorbents have variable efficacy, due in part to their variable binding characteristics. Use of a separate mold inhibitor or mold adsorbent increases the costs associated with animal feed.

A previous study (Doerr et al., Poultry Science, 74(1), 23 (1995)) suggested that Alimet® may reduce the growth of mold in samples of Sabouraud's dextrose broth, potato dextrose agar, or ground corn (19t moisture) treated with either Aspergillus parasiticus, Aspergillus ochraceus, or Fusarium moniliforme, and in ground corn (17.5% moisture) with its naturally occurring fungal flora.

Enhancing the palatability of animal food is an endless endeavor by food manufacturers. Addition of palatants to the food is desirable as a means to increase acceptance by the animals, resulting in improved health of the animal, increased weight gain, etc. Palatants are frequently used in foods for canines, felines, and aquaculture.

### SUMMARY OF THE INVENTION

Accordingly, the present invention provides methods for inhibiting bacteria in animal feed.

In another aspect, the present invention provides methods for inhibiting mold in food, food ingredients, and animal feed compositions.

In another aspect, the present invention provides an anti-bacterial composition comprising a compound of Formula I, as defined herein, for use in inhibiting bacteria in animal feed.

In yet another aspect, the present invention provides an anti-bacterial composition comprising a compound of Formula I and one or more organic acids for use in inhibiting bacteria in animal feed.

Briefly, therefore, the present invention is directed to an anti-bacterial composition comprising a compound of Formula I, as defined herein.

The present invention is also directed to an anti-bacterial composition comprising a compound of Formula I and one or more organic acids.

The present invention is also directed to a method for inhibiting bacteria in animal feed, said method comprising treating said feed with an anti-bacterial composition comprising a compound of Formula I.

The present invention is also directed to a method for inhibiting bacteria in animal feed, said method comprising treating said feed with an anti-bacterial composition comprising a compound of Formula I and one or more organic acids.

The present invention is also directed to a method for inhibiting bacteria in silage, said method comprising treating said silage with an anti-bacterial composition comprising a compound of Formula I.

The present invention is also directed to a method for inhibiting bacteria in silage, said method comprising treating said silage with an anti-bacterial composition comprising a compound of Formula I and one or more organic acids.

The present invention is also directed to a method for inhibiting bacteria in animal feed comprising computing the concentration in said feed of a compound of Formula I necessary to inhibit bacteria present in said animal feed, and applying said compound of Formula I to said feed in a proportion sufficient to achieve said concentration.

The present invention is also directed to a method for inhibiting bacteria in animal feed comprising heat-treating said animal feed, computing the concentration in said feed of a compound of Formula I necessary to inhibit bacteria present in said animal feed, and applying said compound of Formula I to said feed in a proportion sufficient to achieve said concentration.

The present invention is also directed to a method for inhibiting bacteria in animal feed comprising computing the concentration in said feed of said compound of Formula I and another organic acid or mixture of organic acids necessary to inhibit bacteria present in said animal feed, and applying said compound of Formula I and said organic acid or mixture of organic acids to said feed in a proportion sufficient to achieve said concentration.

The present invention is also directed to a method for inhibiting bacteria in animal feed comprising heat-treating said animal feed, computing the concentration in said feed of a compound of Formula I and said organic acid or mixture of organic acids necessary to inhibit bacteria present in said animal feed, and applying said compound of Formula I and said organic acid or mixture of organic acids to said feed in a proportion sufficient to achieve said concentration.

The present invention is also directed to a method of method for inhibiting mold in animal feed comprising directly or indirectly making information available for computing the concentration in said feed of a compound of Formula I necessary to inhibit bacteria present in said animal feed which has been heat-treated, and directly or indirectly making information available for applying said compound of Formula I to said feed in a proportion sufficient to achieve said concentration.

The present invention is also directed to a method of method for inhibiting mold in animal feed comprising directly or indirectly making information available for computing the concentration in said feed of a compound of Formula I and another organic acid or combination of organic acids necessary to inhibit bacteria present in said animal feed, and directly or indirectly making information available for applying said compound of Formula I and said organic acid or combination of organic acids to said feed in a proportion sufficient to achieve said concentration.

The present invention is also directed to a method for inhibiting bacteria in animal feed, said method comprising heat-treating the feed in combination with treating said feed with an anti-bacterial composition comprising a compound of Formula I and comprising one or more organic acids.

The present invention is directed to a method of inhibiting mold in an animal feed composition, the method comprising applying a compound of Formula I to said feed composition, wherein said feed composition comprises corn and soy.

The present invention is also directed to a method for delaying the formation of mold in an animal feed composition, the method comprising applying a compound of Formula I to said feed composition, wherein said feed composition comprises corn and soy.

The present invention is also directed to a method of inhibiting the formation of mold in an animal feed composition, the method comprising applying a compound of Formula I to said feed composition, wherein said feed composition has a moisture content of about 17% or less.

The present invention is also directed to a method for inhibiting mold in silage, said method comprising treating said silage with an anti-fungal composition comprising a compound of Formula I.

The present invention is also directed to a method for inhibiting mold in silage, said method comprising treating said feed with an anti-fungal composition comprising a compound of Formula I and one or more organic acids.

The present invention is also directed to a method for inhibiting the growth of mold in an animal feed composition, the method comprising computing the concentration in said feed composition of a compound of Formula I necessary to inhibit the growth of mold in said feed composition; and applying said compound of Formula I to said feed composition in an amount sufficient to achieve said concentration.

The present invention is also directed to a method for inhibiting the growth of mold in animal feed comprising computing the concentration in said feed of a compound of Formula I necessary to inhibit the growth of mold in said animal feed; and applying said compound of Formula I to said feed in said concentration.

The present invention is also directed to a method for improving the mold resistance of an animal feed composition, the method comprising discontinuing the use of DL-methionine as a feed supplement; computing the concentration in said feed of a compound of Formula I necessary to inhibit the growth of mold in said animal feed; and applying said compound of Formula I to said feed in an amount sufficient to achieve said concentration.

The present invention is also directed to a method for inhibiting mold in animal feed comprising directly or indirectly making information available for computing the concentration in said feed of a compound of Formula I necessary to inhibit mold present in said animal feed; and directly or indirectly making information available for applying said compound of Formula I to said feed in an amount sufficient to achieve said concentration.

The present invention is also directed to method for inhibiting mold in animal feed comprising directly or indirectly making information available for computing the concentration in said feed of a compound of Formula I and another organic acid or mixture of organic acids necessary to inhibit mold present in said animal feed; and directly or indirectly making information available for applying said compound of Formula I and said organic acid or mixture of organic acids to said feed in an amount sufficient to achieve said concentration.

The present invention is also directed to the use of a compound of Formula I in the manufacture of a nutrient composition for inhibiting mold in animal feed by treating said feed with said nutrient composition.

The present invention is also directed to the use of a compound of Formula I and one or more organic acids in the manufacture of a nutrient composition for inhibiting mold in animal feed by treating said feed with said nutrient composition.

The present invention is also directed to a method of inhibiting mold in an animal feed composition, the method comprising monitoring the concentration of methionine supplement in said feed composition, adding additional amounts of said methionine supplement as needed to achieve an anti-mold effective concentration of methionine supplement in said feed composition.

The present invention is also directed to a method of enhancing the palatability of animal food, particularly dog and cat food, and food for aquaculture.

Other objects and features will be in part apparent and in part pointed out hereinafter.

### BRIEF DESCRIPTION OF THE DRAWINGS

FIG. 1A is a graph illustrating the effect of varying doses (0.108, 0.3, and 0.83 g/L) of formic acid and Alimet® at pH 4.5 and 6.75 on the number of colony forming units of S. enteritidis after 4 hours.

FIG. 1B is a graph illustrating the effect of varying doses (0.108, 0.3, and 0.83 g/L) of formic acid and Alimet® at pH 4.5 and 6.75 on the number of colony forming units of E. coli after 4 hours.

FIG. 1C is a graph illustrating the effect of varying doses (0.108, 0.3, and 0.83 g/L) of formic acid and Alimet® at pH 4.5 and 6.75 on the number of colony forming units of L. plantarum after 6 hours.

FIG. 1D is a graph illustrating the effect of varying doses (0.108, 0.3, and 0.83 g/L) of formic acid and Alimet® at pH 4.5 and 6.75 on the number of colony forming units of C. jejuni after 6 hours.

FIG. 2A is a graph illustrating the pH-dependent antibacterial effect of formic acid and Alimet® on the number of colony forming units of S. enteritidis.

FIG. 2B is a graph illustrating the pH-dependent antibacterial effect of formic acid and Alimet® on the number of colony forming units of E. coli.

FIG. 2C is a graph illustrating the pH-dependent antibacterial effect of formic acid and Alimet® on the number of colony forming units of L. plantarum.

FIG. 2D is a graph illustrating the pH-dependent antibacterial effect of formic acid and Alimet® on the number of colony forming units of C. jejuni.

FIG. 3 is a graph illustrating the effect of varying doses (1,3 and 5 g/L) formic acid and Alimet® on the number of colony forming units of S. enteritidis after 4 hours at pH 4.5 and 6.75.

FIGS. 4A and 4B are graphs illustrating the effect of varying doses of a combination of formic acid and Alimet® on the number of colony forming units of S. enteritidis after 4 hours at pH 4.5 and 6.75.

FIG. 5 is a graph comparing the effects of hydrochloric acid, formic acid, lactic acid, and Alimet® on the number of colony forming units of E. coli over time, at pH 4 and 7.3.

FIG. 6 is a graph showing the effect of moisture level on the number of colony forming units of Salmonella in meat meal premix.

FIG. 7 is a graph showing the percent recovery of Salmonella for different levels of Alimet® in meat meal premix containing 20% moisture.

FIG. 8 is a graph showing the effect of Alimet® on the number of colony forming units of Salmonella in meat meal premix containing 20% moisture.

FIG. 9 is a graph illustrating the % CO₂ in the headspace for a starter mash with a moisture level of 16.8% having no DLM or Alimet®; with 0.2% DLM; and with 0.2% Alimet®.

FIG. 10 is a graph illustrating the % CO₂ in the headspace for a starter mash with a moisture level of 14.8% having no DLM or Alimet®; with 0.2% DLM; and with 0.2% Alimet®.

FIG. 11 is a graph illustrating the % CO₂ in the headspace for a starter mash with a moisture level of 12.8% having no DLM or Alimet®; with 0.2% DLM; and with 0.2% Alimet®.

FIG. 12 is a graph illustrating the % CO₂ in the headspace for a starter mash with a moisture level of 14.8% having no DLM or Alimet®; with 0.2% Alimet®; with 2 lb/ton 65% propionic acid plus 0.2% DLM; with 1.5 lb/ton 65% propionic acid plus 0.2% DLM; and with 1.0 lb/ton 65% propionic acid plus 0.2% DLM.

FIG. 13 is a graph illustrating the % CO₂ in the headspace for a starter mash with a moisture level of 14.8% with 2 lb/ton 65% propionic acid; with 1.0 lb/ton 65% propionic acid; and with 1.0 lb/ton 65% propionic acid plus 0.2% Alimet®.

FIG. 14 is a graph illustrating the % CO₂ in the headspace for a starter mash with a moisture level of 14.8% with 2 lb/ton 65% propionic acid; with 1.5 lb/ton 65% propionic acid; and with 1.5 lb/ton 65% propionic acid plus 0.2% Alimet®.

FIG. 15 is a graph illustrating the % CO₂ in the headspace for a starter mash treated with propionic acid and propionic acid plus 0.2% Alimet® with a moisture level of 16.8%, 14.8%, 12.8%.

### DETAILED DESCRIPTION OF THE PREFERRED EMBODIMENTS

A method has been discovered for inhibiting microbes in animal feed, said method comprising treating said feed with a compound of Formula I.

Compounds of Formula I have the following structure:

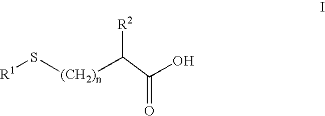

wherein R¹ is an alkyl group having from one to four carbon atoms;

n is an integer from 0 to 2;

R² is selected from the group consisting of hydroxy, amino, —OCOR³, or —NHCOR³;

and wherein R³ is an organic acid derivative;

or a salt thereof.

The term “organic acid derivative” means a derivative of any suitable organic acid resulting from removal of the carboxyl function from the acid. Preferably, the organic acid has from one to eight carbon atoms. Suitable organic acid derivatives include, but are not limited to, derivatives of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid.

Preferably, R¹ is methyl, ethyl, propyl (including n-propyl and isopropyl), or butyl (including n-butyl, sec-butyl, and t-butyl).

In a preferred embodiment, the compound of Formula I is selected from the following list of compounds:

1-hydroxy-1-(methylthio)acetic acid;

1-hydroxy-1-(ethylthio)acetic acid;

1-hydroxy-1-(propylthio)acetic acid;

1-hydroxy-1-(butylthio)acetic acid;

1-amino-1-(methylthio)acetic acid;

1-amino-1-(ethylthio)acetic acid;

1-amino-1-(propylthio)acetic acid;

1-amino-1-(butylthio)acetic acid;

1-carboxy-1-(methylthio)acetic acid;

1-acetyloxy-1-(methylthio)acetic acid;

1-propionyloxy-1-(methylthio)acetic acid;

1-butyryloxy-1-(methylthio)acetic acid;

1-benzoyloxy-1-(methylthio)acetic acid;

1-lactoyloxy-1-(methylthio)acetic acid;

1-[2-carboxy-2-(hydroxy)propionyloxy]-1-(methylthio)acetic acid;

1-[2-carboxy-1-(hydroxy)propionyloxy]-1-(methylthio)acetic acid;

1-[2-carboxy-1,2-(dihydroxy)propionyloxy]-1-(methylthio)acetic acid;

1-[hydroxy(phenyl)acetyl]oxy-1-(methylthio)acetic acid;

1-[2,3-dicarboxy-2-(hydroxy)butyryloxy]-1-(methylthio)acetic acid;

1-[2-carboxy-1-carboxymethyl-1-(hydroxy)propionyloxy]-1-(methylthio)acetic acid;

1-(3-carboxyacryloyl)oxy-1-(methylthio)acetic acid;

1-(2,4-pentadienoyloxy)-1-(methylthio)acetic acid;

1-(2-carboxypropionyloxy)-1-(methylthio)acetic acid;

1-[(4-carboxy)amyloxy]-1-(methylthio)acetic acid;

1-glycoloyloxy-1-(methylthio)acetic acid;

1-glutaroyloxy-1-(methylthio)acetic acid;

1-formylamino-1-(methylthio)acetic acid;

1-acetylamino-1-(methylthio)acetic acid;

1-propionylamino-1-(methylthio)acetic acid;

1-butyrylamino-1-(methylthio)acetic acid;

1-benzoylamino-1-(methylthio)acetic acid;

1-lactoylamino-1-(methylthio)acetic acid;

1-[2-carboxy-2-(hydroxy)propionylamino]-1-(methylthio)acetic acid;

1-[2-carboxy-1-(hydroxy)propionylamino]-1-(methylthio)acetic acid;

1-[2-carboxy-1,2-(dihydroxy)propionylamino]-1-(methylthio)acetic acid;

1-[hydroxy(phenyl)acetyl]amino-1-(methylthio)acetic acid;

1-[2,3-dicarboxy-2-(hydroxy)butyrylamino]-1-(methylthio)acetic acid;

1-[2-carboxy-1-carboxymethyl-1-(hydroxy)propionylamino]-1-(methylthio)acetic acid;

1-(3-carboxyacryloyl)amino-1-(methylthio)acetic acid;

1-(2,4-pentadienoylamino)-1-(methylthio)acetic acid;

1-(2-carboxypropionylamino)-1-(methylthio)acetic acid;

1-[(4-carboxy)amylamino]-1-(methylthio)acetic acid;

1-glycoloylamino-1-(methylthio)acetic acid;

1-glutaroylamino-1-(methylthio)acetic acid;

1-carboxy-(ethylthio)acetic acid;

1-acetyloxy-(ethylthio)acetic acid;

1-propionyloxy-(ethylthio)acetic acid;

1-butyryloxy-(ethylthio)acetic acid;

1-benzoyloxy-(ethylthio)acetic acid;

1-lactoyloxy-(ethylthio)acetic acid;

1-[2-carboxy-2-(hydroxy)propionyloxy]-(ethylthio)acetic acid;

1-[2-carboxy-1-(hydroxy)propionyloxy]-(ethylthio)acetic acid;

1-[2-carboxy-1,2-(dihydroxy)propionyloxy]-(ethylthio)acetic acid;

1-[hydroxy(phenyl)acetyl]oxy-(ethylthio)acetic acid;

1-[2,3-dicarboxy-2-(hydroxy)butyryloxy]-(ethylthio)acetic acid;

1-[2-carboxy-1-carboxymethyl-1-(hydroxy)propionyloxy]-(ethylthio)acetic acid;

1-(3-carboxyacryloyl)oxy-(ethylthio)acetic acid;

1-(2,4-pentadienoyloxy)-(ethylthio)acetic acid;

1-(2-carboxypropionyloxy)-(ethylthio)acetic acid;

1-[(4-carboxy)amyloxy]-(ethylthio)acetic acid;

1-glycoloyloxy-(ethylthio)acetic acid;

1-glutaroyloxy-(ethylthio)acetic acid;

1-formylamino-(ethylthio)acetic acid;

1-acetylamino-(ethylthio)acetic acid;

1-propionylamino-(ethylthio)acetic acid;

1-butyrylamino-(ethylthio)acetic acid;

1-benzoylamino-(ethylthio)acetic acid;

1-lactoylamino-(ethylthio)acetic acid;

1-[2-carboxy-2-(hydroxy)propionylamino]-(ethylthio)acetic acid;

1-[2-carboxy-1-(hydroxy)propionylamino]-(ethylthio)acetic acid;

1-[2-carboxy-1,2-(dihydroxy)propionylamino]-(ethylthio)acetic acid;

1-[hydroxy(phenyl)acetyl]amino-(ethylthio)acetic acid;

1-[2,3-dicarboxy-2-(hydroxy)butyrylamino]-(ethylthio)acetic acid;

1-[2-carboxy-1-carboxymethyl-1-(hydroxy)propionylamino]-(ethylthio)acetic acid;

1-(3-carboxyacryloyl)amino-(ethylthio)acetic acid;

1-(2,4-pentadienoylamino)-(ethylthio)acetic acid;

1-(2-carboxypropionylamino)-(ethylthio)acetic acid;

1-[(4-carboxy)amylamino]-(ethylthio)acetic acid;

1-glycoloylamino-(ethylthio)acetic acid;

1-glutaroylamino-(ethylthio)acetic acid;

1-carboxy-(propylthio)acetic acid;

1-acetyloxy-(propylthio)acetic acid;

1-propionyloxy-(propylthio)acetic acid;

1-butyryloxy-(propylthio)acetic acid;

1-benzoyloxy-(propylthio)acetic acid;

1-lactoyloxy-(propylthio)acetic acid;

1-[2-carboxy-2-(hydroxy)propionyloxyl-(propylthio)acetic acid;

1-[2-carboxy-1-(hydroxy)propionyloxy]-(propylthio)acetic acid;

1-[2-carboxy-1,2-(dihydroxy)propionyloxy]-(propylthio)acetic acid;

1-[hydroxy(phenyl)acetyl]oxy-(propylthio)acetic acid;

1-[2,3-dicarboxy-2-(hydroxy)butyryloxy]-(propylthio)acetic acid;

1-[2-carboxy-1-carboxymethyl-1-(hydroxy)propionyloxy]-(propylthio)acetic acid;

1-(3-carboxyacryloyl)oxy-(propylthio)acetic acid;

1-(2,4-pentadienoyloxy)-(propylthio)acetic acid;

1-(2-carboxypropionyloxy)-(propylthio)acetic acid;

1-[(4-carboxy)amyloxy]-(propylthio)acetic acid;

1-glycoloyloxy-(propylthio)acetic acid;

1-glutaroyloxy-(propylthio)acetic acid;

1-formylamino-(propylthio)acetic acid;

1-acetylamino-(propylthio)acetic acid;

1-propionylamino-(propylthio)acetic acid;

1-butyrylamino-(propylthio)acetic acid;

1-benzoylamino-(propylthio)acetic acid;

1-lactoylamino-(propylthio)acetic acid;

1-[2-carboxy-2-(hydroxy)propionylamino]-(propylthio)acetic acid;

1-[2-carboxy-1-(hydroxy)propionylamino]-(propylthio)acetic acid;

1-[2-carboxy-1,2-(dihydroxy)propionylamino]-(propylthio)acetic acid;

1-[hydroxy(phenyl)acetyl]amino-(propylthio)acetic acid;

1-[2,3-dicarboxy-2-(hydroxy)butyrylamino]-(propylthio)acetic acid;

1-[2-carboxy-1-carboxymethyl-1-(hydroxy)propionylamino]-(propylthio)acetic acid;

1-(3-carboxyacryloyl)amino-(propylthio)acetic acid;

1-(2,4-pentadienoylamino)-(propylthio)acetic acid;

1-(2-carboxypropionylamino)-(propylthio)acetic acid;

1-[(4-carboxy)amylamino]-(propylthio)acetic acid;

1-glycoloylamino-(propylthio)acetic acid;

1-glutaroylamino-(propylthio)acetic acid;

1-carboxy-(butylthio)acetic acid;

1-acetyloxy-(butylthio)acetic acid;

1-propionyloxy-(butylthio)acetic acid;

1-butyryloxy-(butylthio)acetic acid;

1-benzoyloxy-(butylthio)acetic acid;

1-lactoyloxy-(butylthio)acetic acid;

1-[2-carboxy-2-(hydroxy)propionyloxy]-(butylthio)acetic acid;

1-[2-carboxy-1-(hydroxy)propionyloxy]-(butylthio)acetic acid;

1-[2-carboxy-1,2-(dihydroxy)propionyloxy]-(butylthio)acetic acid;

1-[hydroxy(phenyl)acetyl]oxy-(butylthio)acetic acid;

1-[2,3-dicarboxy-2-(hydroxy)butyryloxy]-(butylthio)acetic acid;

1-[2-carboxy-1-carboxymethyl-1-(hydroxy)propionyloxy]-(butylthio)acetic acid;

1-(3-carboxyacryloyl)oxy-(butylthio)acetic acid;

1-(2,4-pentadienoyloxy)-(butylthio)acetic acid;

1-(2-carboxypropionyloxy)-(butylthio)acetic acid;

1-[(4-carboxy)amyloxy]-(butylthio)acetic acid;

1-glycoloyloxy-(butylthio)acetic acid;

1-glutaroyloxy-(butylthio)acetic acid;

1-formylamino-(butylthio)acetic acid;

1-acetylamino-(butylthio)acetic acid;

1-propionylamino-(butylthio)acetic acid;

1-butyrylamino-(butylthio)acetic acid;

1-benzoylamino-(butylthio)acetic acid;

1-lactoylamino-(butylthio)acetic acid;

1-[2-carboxy-2-(hydroxy)propionylamino]-(butylthio)acetic acid;

1-[2-carboxy-1-(hydroxy)propionylamino]-(butylthio)acetic acid;

1-[2-carboxy-1,2-(dihydroxy)propionylamino]-(butylthio)acetic acid;

1-[hydroxy(phenyl)acetyl]amino-(butylthio)acetic acid;

1-[2,3-dicarboxy-2-(hydroxy)butyrylamino]-(butylthio)acetic acid;

1-[2-carboxy-1-carboxymethyl-1-(hydroxy)propionylamino]-(butylthio)acetic acid;

1-(3-carboxyacryloyl)amino-(butylthio)acetic acid;

1-(2,4-pentadienoylamino)-(butylthio)acetic acid;

1-(2-carboxypropionylamino)-(butylthio)acetic acid;

1-[(4-carboxy)amylamino]-(butylthio)acetic acid;

1-glycoloylamino-(butylthio)acetic acid;

1-glutaroylamino-(butylthio)acetic acid;

2-hydroxy-3-(methylthio)propanoic acid;

2-hydroxy-3-(ethylthio)propanoic acid;

2-hydroxy-3-(propylthio)propanoic acid;

2-hydroxy-3-(butylthio)propanoic acid;

2-amino-3-(methylthio)propanoic acid;

2-amino-3-(ethylthio)propanoic acid;

2-amino-3-(propylthio)propanoic acid;

2-amino-3-(butylthio)propanoic acid;

2-carboxy-3-(methylthio)propanoic acid;

2-acetyloxy-3-(methylthio)propanoic acid;

2-propionyloxy-3-(methylthio)propanoic acid;

2-butyryloxy-3-(methylthio)propanoic acid;

2-benzoyloxy-3-(methylthio)propanoic acid;

2-lactoyloxy-3-(methylthio)propanoic acid;

2-[2-carboxy-2-(hydroxy)propionyloxy]-3-(methylthio)propanoic acid;

2-[2-carboxy-1-(hydroxy)propionyloxy]-3-(methylthio)propanoic acid;

2-[2-carboxy-1,2-(dihydroxy)propionyloxy]-3-(methylthio)propanoic acid;

2-[hydroxy(phenyl)acetyl]oxy-3-(methylthio)propanoic acid;

2-[2,3-dicarboxy-2-(hydroxy)butyryloxy]-3-(methylthio)propanoic acid;

2-[2-carboxy-1-carboxymethyl-1-(hydroxy)propionyloxy]-3-(methylthio)propanoic acid;

2-(3-carboxyacryloyl)oxy-3-(methylthio)propanoic acid;

2-(2,4-pentadienoyloxy)-3-(methylthio)propanoic acid;

2-(2-carboxypropionyloxy)-3-(methylthio)propanoic acid;

2-[(4-carboxy)amyloxy]-3-(methylthio)propanoic acid;

2-glycoloyloxy-3-(methylthio)propanoic acid;

2-glutaroyloxy-3-(methylthio)propanoic acid;

2-formylamino-3-(methylthio)propanoic acid;

2-acetylamino-3-(methylthio)propanoic acid;

2-propionylamino-3-(methylthio)propanoic acid;

2-butyrylamino-3-(methylthio)propanoic acid;

2-benzoylamino-3-(methylthio)propanoic acid;

2-lactoylamino-3-(methylthio)propanoic acid;

2-[2-carboxy-2-(hydroxy)propionylamino]-3-(methylthio)propanoic acid;

2-[2-carboxy-1-(hydroxy)propionylamino]-3-(methylthio)propanoic acid;

2-[2-carboxy-1,2-(dihydroxy)propionylamino]-3-(methylthio)propanoic acid;

2-[hydroxy(phenyl)acetyl]amino-3-(methylthio)propanoic acid;

2-[2,3-dicarboxy-2-(hydroxy)butyrylamino]-3-(methylthio)propanoic acid;

2-[2-carboxy-1-carboxymethyl-1-(hydroxy)propionylamino]-3-(methylthio)propanoic acid;

2-(3-carboxyacryloyl)amino-3-(methylthio)propanoic acid;

2-(2,4-pentadienoylamino)-3-(methylthio)propanoic acid;

2-(2-carboxypropionylamino)-3-(methylthio)propanoic acid;

2-[(4-carboxy)amylamino]-3-(methylthio)propanoic acid;

2-glycoloylamino-3-(methylthio)propanoic acid;

2-glutaroylamino-3-(methylthio)propanoic acid;

2-carboxy-3-(ethylthio)propanoic acid;

2-acetyloxy-3-(ethylthio)propanoic acid;

2-propionyloxy-3-(ethylthio)propanoic acid;

2-butyryloxy-3-(ethylthio)propanoic acid;

2-benzoyloxy-3-(ethylthio)propanoic acid;

2-lactoyloxy-3-(ethylthio)propanoic acid;

2-[2-carboxy-2-(hydroxy)propionyloxy]-3-(ethylthio)propanoic acid;

2-[2-carboxy-1-(hydroxy)propionyloxy]-3-(ethylthio)propanoic acid;

2-[2-carboxy-1,2-(dihydroxy)propionyloxy]-3-(ethylthio)propanoic acid;

2-[hydroxy(phenyl)acetyl]oxy-3-(ethylthio)propanoic acid;

2-[2,3-dicarboxy-2-(hydroxy)butyryloxy]-3-(ethylthio)propanoic acid;

2-[2-carboxy-1-carboxymethyl-1-(hydroxy)propionyloxy]-3-(ethylthio)propanoic acid;

2-(3-carboxyacryloyl)oxy-3-(ethylthio)propanoic acid;

2-(2,4-pentadienoyloxy)-3-(ethylthio)propanoic acid;

2-(2-carboxypropionyloxy)-3-(ethylthio)propanoic acid;

2-[(4-carboxy)amyloxy]-3-(ethylthio)propanoic acid;

2-glycoloyloxy-3-(ethylthio)propanoic acid;

2-glutaroyloxy-3-(ethylthio)propanoic acid;

2-formylamino-3-(ethylthio)propanoic acid;

2-acetylamino-3-(ethylthio)propanoic acid;

2-propionylamino-3-(ethylthio)propanoic acid;

2-butyrylamino-3-(ethylthio)propanoic acid;

2-benzoylamino-3-(ethylthio)propanoic acid;

2-lactoylamino-3-(ethylthio)propanoic acid;

2-[2-carboxy-2-(hydroxy)propionylamino]-3-(ethylthio)propanoic acid;

2-[2-carboxy-1-(hydroxy)propionylamino]-3-(ethylthio)propanoic acid;

2-[2-carboxy-1,2-(dihydroxy)propionylamino]-3-(ethylthio)propanoic acid;

2-[hydroxy(phenyl)acetyl]amino-3-(ethylthio)propanoic acid;

2-[2,3-dicarboxy-2-(hydroxy)butyrylamino]-3-(ethylthio)propanoic acid;

2-[2-carboxy-1-carboxymethyl-1-(hydroxy)propionylamino]-3-(ethylthio)propanoic acid;

2-(3-carboxyacryloyl)amino-3-(ethylthio)propanoic acid;

2-(2,4-pentadienoylamino)-3-(ethylthio)propanoic acid;

2-(2-carboxypropionylamino)-3-(ethylthio)propanoic acid;

2-[(4-carboxy)amylamino]-3-(ethylthio)propanoic acid;

2-glycoloylamino-3-(ethylthio)propanoic acid;

2-glutaroylamino-3-(ethylthio)propanoic acid;

2-carboxy-3-(propylthio)propanoic acid;

2-acetyloxy-3-(propylthio)propanoic acid;

2-propionyloxy-3-(propylthio)propanoic acid;

2-butyryloxy-3-(propylthio)propanoic acid;

2-benzoyloxy-3-(propylthio)propanoic acid;

2-lactoyloxy-3-(propylthio)propanoic acid;

2-[2-carboxy-2-(hydroxy)propionyloxy]-3-(propylthio)propanoic acid;

2-[2-carboxy-1-(hydroxy)propionyloxy]-3-(propylthio)propanoic acid;

2-[2-carboxy-1,2-(dihydroxy)propionyloxy]-3-(propylthio)propanoic acid;

2-[hydroxy(phenyl)acetyl]oxy-3-(propylthio)propanoic acid;

2-[2,3-dicarboxy-2-(hydroxy)butyryloxy]-3-(propylthio)propanoic acid;

2-[2-carboxy-1-carboxymethyl-1-(hydroxy)propionyloxy]-3-(propylthio)propanoic acid;

2-(3-carboxyacryloyl)oxy-3-(propylthio)propanoic acid;

2-(2,4-pentadienoyloxy)-3-(propylthio)propanoic acid;

2-(2-carboxypropionyloxy)-3-(propylthio)propanoic acid;

2-[(4-carboxy)amyloxy]-3-(propylthio)propanoic acid;

2-glycoloyloxy-3-(propylthio)propanoic acid;

2-glutaroyloxy-3-(propylthio)propanoic acid;

2-formylamino-3-(propylthio)propanoic acid;

2-acetylamino-3-(propylthio)propanoic acid;

2-propionylamino-3-(propylthio)propanoic acid;

2-butyrylamino-3-(propylthio)propanoic acid;

2-benzoylamino-3-(propylthio)propanoic acid;

2-lactoylamino-3-(propylthio)propanoic acid;

2-[2-carboxy-2-(hydroxy)propionylamino]-3-(propylthio)propanoic acid;

2-[2-carboxy-1-(hydroxy)propionylamino]-3-(propylthio)propanoic acid;

2-[2-carboxy-1,2-(dihydroxy)propionylamino]-3-(propylthio)propanoic acid;

2-[hydroxy(phenyl)acetyl]amino-3-(propylthio)propanoic acid;

2-[2,3-dicarboxy-2-(hydroxy)butyrylamino]-3-(propylthio)propanoic acid;

2-[2-carboxy-1-carboxymethyl-1-(hydroxy)propionylamino]-3-(propylthio)propanoic acid;

2-(3-carboxyacryloyl)amino-3-(propylthio)propanoic acid;

2-(2,4-pentadienoylamino)-3-(propylthio)propanoic acid;

2-(2-carboxypropionylamino)-3-(propylthio)propanoic acid;

2-[(4-carboxy)amylamino]-3-(propylthio)propanoic acid;

2-glycoloylamino-3-(propylthio)propanoic acid;

2-glutaroylamino-3-(propylthio)propanoic acid;

2-carboxy-3-(butylthio)propanoic acid;

2-acetyloxy-3-(butylthio)propanoic acid;

2-propionyloxy-3-(butylthio)propanoic acid;

2-butyryloxy-3-(butylthio)propanoic acid;

2-benzoyloxy-3-(butylthio)propanoic acid;

2-lactoyloxy-3-(butylthio)propanoic acid;

2-[2-carboxy-2-(hydroxy)propionyloxy]-3-(butylthio)propanoic acid;

2-[2-carboxy-1-(hydroxy)propionyloxy]-3-(butylthio)propanoic acid;

2-[2-carboxy-1,2-(dihydroxy)propionyloxy]-3-(butylthio)propanoic acid;

2-[hydroxy(phenyl)acetyl]oxy-3-(butylthio)propanoic acid;

2-[2,3-dicarboxy-2-(hydroxy)butyryloxy]-3-(butylthio)propanoic acid;

2-[2-carboxy-1-carboxymethyl-1-(hydroxy)propionyloxy]-3-(butylthio)propanoic acid;

2-(3-carboxyacryloyl)oxy-3-(butylthio)propanoic acid;

2-(2,4-pentadienoyloxy)-3-(butylthio)propanoic acid;

2-(2-carboxypropionyloxy)-3-(butylthio)propanoic acid;

2-[(4-carboxy)amyloxy]-3-(butylthio)propanoic acid;

2-glycoloyloxy-3-(butylthio)propanoic acid;

2-glutaroyloxy-3-(butylthio)propanoic acid;

2-formylamino-3-(butylthio)propanoic acid;

2-acetylamino-3-(butylthio)propanoic acid;

2-propionylamino-3-(butylthio)propanoic acid;

2-butyrylamino-3-(butylthio)propanoic acid;

2-benzoylamino-3-(butylthio)propanoic acid;

2-lactoylamino-3-(butylthio)propanoic acid;

2-[2-carboxy-2-(hydroxy)propionylamino]-3-(butylthio)propanoic acid;

2-[2-carboxy-1-(hydroxy)propionylamino]-3-(butylthio)propanoic acid;

2-[2-carboxy-1,2-(dihydroxy)propionylamino]-3-(butylthio)propanoic acid;

2-[hydroxy(phenyl)acetyl]amino-3-(butylthio)propanoic acid;

2-[2,3-dicarboxy-2-(hydroxy)butyrylamino]-3-(butylthio)propanoic acid;

2-[2-carboxy-1-carboxymethyl-1-(hydroxy)propionylamino]-3-(butylthio)propanoic acid;

2-(3-carboxyacryloyl)amino-3-(butylthio)propanoic acid;

2-(2,4-pentadienoylamino)-3-(butylthio)propanoic acid;

2-(2-carboxypropionylamino)-3-(butylthio)propanoic acid;

2-[(4-carboxy)amylamino]-3-(butylthio)propanoic acid;

2-glycoloylamino-3-(butylthio)propanoic acid;

2-glutaroylamino-3-(butylthio)propanoic acid;

2-hydroxy-4-(methylthio)butanoic acid;

2-hydroxy-4-(ethylthio)butanoic acid;

2-hydroxy-4-(propylthio)butanoic acid;

2-hydroxy-4-(butylthio)butanoic acid;

2-amino-4-(methylthio)butanoic acid;

2-amino-4-(ethylthio)butanoic acid;

2-amino-4-(propylthio)butanoic acid;

2-amino-4-(butylthio)butanoic acid;

2-carboxy-4-(methylthio)butanoic acid;

2-acetyloxy-4-(methylthio)butanoic acid;

2-propionyloxy-4-(methylthio)butanoic acid;

2-butyryloxy-4-(methylthio)butanoic acid;

2-benzoyloxy-4-(methylthio)butanoic acid;

2-lactoyloxy-4-(methylthio)butanoic acid;

2-[2-carboxy-2-(hydroxy)propionyloxy]-4-(methylthio)butanoic acid;

2-[2-carboxy-1-(hydroxy)propionyloxy]-4-(methylthio)butanoic acid;

2-[2-carboxy-1,2-(dihydroxy)propionyloxy]-4-(methylthio)butanoic acid;

2-[hydroxy(phenyl)acetyl]oxy-4-(methylthio)butanoic acid;

2-[2,3-dicarboxy-2-(hydroxy)butyryloxy]-4-(methylthio)butanoic acid;

2-[2-carboxy-1-carboxymethyl-1-(hydroxy)propionyloxy]-4-(methylthio)butanoic acid;

2-(3-carboxyacryloyl)oxy-4-(methylthio)butanoic acid;

2-(2,4-pentadienoyloxy)-4-(methylthio)butanoic acid;

2-(2-carboxypropionyloxy)-4-(methylthio)butanoic acid;

2-[(4-carboxy)amyloxy]-4-(methylthio)butanoic acid;

2-glycoloyloxy-4-(methylthio)butanoic acid;

2-glutaroyloxy-4-(methylthio)butanoic acid;

2-formylamino-4-(methylthio)butanoic acid;

2-acetylamino-4-(methylthio)butanoic acid;

2-propionylamino-4-(methylthio)butanoic acid;

2-butyrylamino-4-(methylthio)butanoic acid;

2-benzoylamino-4-(methylthio)butanoic acid;

2-lactoylamino-4-(methylthio)butanoic acid;

2-[2-carboxy-2-(hydroxy)propionylamino]-4-(methylthio)butanoic acid;

2-[2-carboxy-1-(hydroxy)propionylamino]-4-(methylthio)butanoic acid;

2-[2-carboxy-1,2-(dihydroxy)propionylamino]-4-(methylthio)butanoic acid;

2-[hydroxy(phenyl)acetyl]amino-4-(methylthio)butanoic acid;

2-[2,3-dicarboxy-2-(hydroxy)butyrylamino]-4-(methylthio)butanoic acid;

2-[2-carboxy-1-carboxymethyl-1-(hydroxy)propionylamino]-4-(methylthio)butanoic acid;

2-(3-carboxyacryloyl)amino-4-(methylthio)butanoic acid;

2-(2,4-pentadienoylamino)-4-(methylthio)butanoic acid;

2-(2-carboxypropionylamino)-4-(methylthio)butanoic acid;

2-[(4-carboxy)amylamino]-4-(methylthio)butanoic acid;

2-glycoloylamino-4-(methylthio)butanoic acid;

2-glutaroylamino-4-(methylthio)butanoic acid;

2-carboxy-4-(ethylthio)butanoic acid;

2-acetyloxy-4-(ethylthio)butanoic acid;

2-propionyloxy-4-(ethylthio)butanoic acid;

2-butyryloxy-4-(ethylthio)butanoic acid;

2-benzoyloxy-4-(ethylthio)butanoic acid;

2-lactoyloxy-4-(ethylthio)butanoic acid;

2-[2-carboxy-2-(hydroxy)propionyloxy]-4-(ethylthio)butanoic acid;

2-[2-carboxy-1-(hydroxy)propionyloxy]-4-(ethylthio)butanoic acid;

2-[2-carboxy-1,2-(dihydroxy)propionyloxy]-4-(ethylthio)butanoic acid;

2-[hydroxy(phenyl)acetyl]oxy-4-(ethylthio)butanoic acid;

2-[2,3-dicarboxy-2-(hydroxy)butyryloxy]-4-(ethylthio)butanoic acid;

2-[2-carboxy-1-carboxymethyl-1-(hydroxy)propionyloxy]-4-(ethylthio)butanoic acid;

2-(3-carboxyacryloyl)oxy-4-(ethylthio)butanoic acid;

2-(2,4-pentadienoyloxy)-4-(ethylthio)butanoic acid;

2-(2-carboxypropionyloxy)-4-(ethylthio)butanoic acid;

2-[(4-carboxy)amyloxy]-4-(ethylthio)butanoic acid;

2-glycoloyloxy-4-(ethylthio)butanoic acid;

2-glutaroyloxy-4-(ethylthio)butanoic acid;

2-formylamino-4-(ethylthio)butanoic acid;

2-acetylamino-4-(ethylthio)butanoic acid;

2-propionylamino-4-(ethylthio)butanoic acid;

2-butyrylamino-4-(ethylthio)butanoic acid;

2-benzoylamino-4-(ethylthio)butanoic acid;

2-lactoylamino-4-(ethylthio)butanoic acid;

2-[2-carboxy-2-(hydroxy)propionylamino]-4-(ethylthio)butanoic acid;

2-[2-carboxy-1-(hydroxy)propionylamino]-4-(ethylthio)butanoic acid;

2-[2-carboxy-1,2-(dihydroxy)propionylamino]-4-(ethylthio)butanoic acid;

2-[hydroxy(phenyl)acetyl]amino-4-(ethylthio)butanoic acid;

2-[2,3-dicarboxy-2-(hydroxy)butyrylamino]-4-(ethylthio)butanoic acid;

2-[2-carboxy-1-carboxymethyl-1-(hydroxy)propionylamino]-4-(ethylthio)butanoic acid;

2-(3-carboxyacryloyl)amino-4-(ethylthio)butanoic acid;

2-(2,4-pentadienoylamino)-4-(ethylthio)butanoic acid;

2-(2-carboxypropionylamino)-4-(ethylthio)butanoic acid;

2-[(4-carboxy)amylamino]-4-(ethylthio)butanoic acid;

2-glycoloylamino-4-(ethylthio)butanoic acid;

2-glutaroylamino-4-(ethylthio)butanoic acid;

2-carboxy-4-(propylthio)butanoic acid;

2-acetyloxy-4-(propylthio)butanoic acid;

2-propionyloxy-4-(propylthio)butanoic acid;

2-butyryloxy-4-(propylthio)butanoic acid;

2-benzoyloxy-4-(propylthio)butanoic acid;

2-lactoyloxy-4-(propylthio)butanoic acid;

2-[2-carboxy-2-(hydroxy)propionyloxy]-4-(propylthio)butanoic acid;

2-[2-carboxy-1-(hydroxy)propionyloxy]-4-(propylthio)butanoic acid;

2-[2-carboxy-1,2-(dihydroxy)propionyloxy]-4-(propylthio)butanoic acid;

2-[hydroxy(phenyl)acetyl]oxy-4-(propylthio)butanoic acid;

2-[2,3-dicarboxy-2-(hydroxy)butyryloxy]-4-(propylthio)butanoic acid;

2-[2-carboxy-1-carboxymethyl-1-(hydroxy)propionyloxy]-4-(propylthio)butanoic acid;

2-(3-carboxyacryloyl)oxy-4-(propylthio)butanoic acid;

2-(2,4-pentadienoyloxy)-4-(propylthio)butanoic acid;

2-(2-carboxypropionyloxy)-4-(propylthio)butanoic acid;

2-[(4-carboxy)amyloxy]-4-(propylthio)butanoic acid;

2-glycoloyloxy-4-(propylthio)butanoic acid;

2-glutaroyloxy-4-(propylthio)butanoic acid;

2-formylamino-4-(propylthio)butanoic acid;

2-acetylamino-4-(propylthio)butanoic acid;

2-propionylamino-4-(propylthio)butanoic acid;

2-butyrylamino-4-(propylthio)butanoic acid;

2-benzoylamino-4-(propylthio)butanoic acid;

2-lactoylamino-4-(propylthio)butanoic acid;

2-[2-carboxy-2-(hydroxy)propionylamino]-4-(propylthio)butanoic acid;

2-[2-carboxy-1-(hydroxy)propionylamino]-4-(propylthio)butanoic acid;

2-[2-carboxy-1,2-(dihydroxy)propionylamino]-4-(propylthio)butanoic acid;

2-[hydroxy(phenyl)acetyl]amino-4-(propylthio)butanoic acid;

2-[2,3-dicarboxy-2-(hydroxy)butyrylamino]-4-(propylthio)butanoic acid;

2-[2-carboxy-1-carboxymethyl-1-(hydroxy)propionylamino]-4-(propylthio)butanoic acid;

2-(3-carboxyacryloyl)amino-4-(propylthio)butanoic acid;

2-(2,4-pentadienoylamino)-4-(propylthio)butanoic acid;

2-(2-carboxypropionylamino)-4-(propylthio)butanoic acid;

2-[(4-carboxy)amylamino]-4-(propylthio)butanoic acid;

2-glycoloylamino-4-(propylthio)butanoic acid;

2-glutaroylamino-4-(propylthio)butanoic acid;

2-carboxy-4-(butylthio)butanoic acid;

2-acetyloxy-4-(butylthio)butanoic acid;

2-propionyloxy-4-(butylthio)butanoic acid;

2-butyryloxy-4-(butylthio)butanoic acid;

2-benzoyloxy-4-(butylthio)butanoic acid;

2-lactoyloxy-4-(butylthio)butanoic acid;

2-[2-carboxy-2-(hydroxy)propionyloxy]-4-(butylthio)butanoic acid;

2-[2-carboxy-1-(hydroxy)propionyloxy]-4-(butylthio)butanoic acid;

2-[2-carboxy-1,2-(dihydroxy)propionyloxy]-4-(butylthio)butanoic acid;

2-[hydroxy(phenyl)acetyl]oxy-4-(butylthio)butanoic acid;

2-[2,3-dicarboxy-2-(hydroxy)butyryloxy]-4-(butylthio)butanoic acid;

2-[2-carboxy-1-carboxymethyl-1-(hydroxy)propionyloxy]-4-(butylthio)butanoic acid;

2-(3-carboxyacryloyl)oxy-4-(butylthio)butanoic acid;

2-(2,4-pentadienoyloxy)-4-(butylthio)butanoic acid;

2-(2-carboxypropionyloxy)-4-(butylthio)butanoic acid;

2-[(4-carboxy)amyloxy]-4-(butylthio)butanoic acid;

2-glycoloyloxy-4-(butylthio)butanoic acid;

2-glutaroyloxy-4-(butylthio)butanoic acid;

2-formylamino-4-(butylthio)butanoic acid;

2-acetylamino-4-(butylthio)butanoic acid;

2-propionylamino-4-(butylthio)butanoic acid;

2-butyrylamino-4-(butylthio)butanoic acid;

2-benzoylamino-4-(butylthio)butanoic acid;

2-lactoylamino-4-(butylthio)butanoic acid;

2-[2-carboxy-2-(hydroxy)propionylamino]-4-(butylthio)butanoic acid;

2-[2-carboxy-1-(hydroxy)propionylamino]-4-(butylthio)butanoic acid;

2-[2-carboxy-1,2-(dihydroxy)propionylamino]-4-(butylthio)butanoic acid;

2-[hydroxy(phenyl)acetyl]amino-4-(butylthio)butanoic acid;

2-12,3-dicarboxy-2-(hydroxy)butyrylamino]-4-(butylthio)butanoic acid;

2-[2-carboxy-1-carboxymethyl-1-(hydroxy)propionylamino]-4-(butylthio)butanoic acid;

2-(3-carboxyacryloyl)amino-4-(butylthio)butanoic acid;

2-(2,4-pentadienoylamino)-4-(butylthio)butanoic acid;

2-(2-carboxypropionylamino)-4-(butylthio)butanoic acid;

2-[(4-carboxy)amylamino]-4-(butylthio)butanoic acid;

2-glycoloylamino-4-(butylthio)butanoic acid; and

2-glutaroylamino-4-(butylthio)butanoic acid.

In a more preferred embodiment, the compound of Formula I is selected from the group of compounds wherein R¹ is methyl; n is 2; R² is hydroxy or —OCOR³; and R³ is a derivative of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, or glutaric acid. In an even more preferred embodiment, the compound of Formula I is selected from the group of compounds wherein R¹ is methyl; n is 2; R² is hydroxy or —OCOR³; and R³ is a derivative of formic acid, propionic acid, butyric acid, lactic acid, citric acid, or fumaric acid.

Representative salts of the compound of Formula I include the ammonium, magnesium, calcium, lithium, sodium, potassium, selenium, iron, copper, and zinc salts. In a preferred embodiment, the compound of Formula I is in the form of the calcium salt. Representative amides include methylamide, dimethylamide, ethylmethylamide, butylamide, dibutylamide, butylmethylamide, alkyl ester of N-acyl methionates (e.g., alkyl N-acetyl methionates. Representative esters include the methyl, ethyl, n-propyl, isopropyl, butyl esters, namely n-butyl, sec-butyl, isobutyl, and t-butyl esters, pentyl esters and hexyl esters, especially n-pentyl, isopentyl, n-hexyl and isohexyl esters.

In various preferred embodiment, the compound of Formula I is 2-hydroxy-4-(methylthio)butanoic acid (HMBA) or a salt, amide or ester thereof. In still more preferred embodiments, the compound of Formula I is HMBA.

Preferably, the concentration of the compound of Formula I in the feed compositions described herein is between about 0.01% and about 5%. In various preferred embodiments, the concentration is between 0.01% and about 4%; between 0.02% and about 3%; between 0.03% and about 2%; between 0.04% and about 1%; between about 0.05% and about 0.6%; and between about 0.06% and about 0.525%. In various particularly preferred embodiments, the concentration is about 0.075%; about 0.125%; about 0.15%; about 0.225%; about 0.25%; about 0.3%; about 0.375%; and about 0.5%.

In another embodiment of the present invention, the methods of inhibiting microbes in animal feed comprises treating said feed with a compound of Formula I and one or more organic acids. Preferably, the organic acid has a pKₐ<5.5. In one embodiment, the organic acid is a carboxyl-substituted hydrocarbon moiety. The hydrocarbon moiety may be further substituted by one or more substituents such as halogen; oxygen-containing groups such as alkoxy, aryloxy, hydroxy, protected hydroxy, keto, acyl, acyloxy; nitrogen-containing groups such as nitro, amino, amido, cyano; and sulfur-containing groups such as thiol, thioalkyl, and sulfonyl. In a preferred embodiment, said organic acids are selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or combinations thereof. In one embodiment, the organic acid is formic acid, propionic acid, butyric acid, lactic acid, or combinations thereof.

Preferably, the combined concentration of the compound of Formula I and the organic acid or mixture of organic acids in the food compositions described herein is between about 0.01% and about 5%. In various preferred embodiments, the combined concentration is between about 0.015% and about 4%; between about 0.02% and about 3%; between about 0.05% and about 2.5%; between about 0.075% and about 2%; between about 0.1% and about 1.5%; between about 0.15% and about 1%; between about 0.4% and about 0.9%; between about 0.5% and about 0.8%; between about 0.01% and about 5%; between about 0.01% and about 4.5%; between about 0.05% and about 4%; between about 0.08% and about 3%; between about 0.1% and about 2.5%; between about 0.01% and about 0.8%; between about 0.01% and about 0.5%; between about 0.05% and about 0.6%; and between about 0.06% and about 0.525%.

In various other preferred embodiments, the concentration of said compound of Formula I and said organic acid in the food compositions described herein is as follows:

| Concentration of the | Concentration of the |
|---|---|
| compound of Formula I | organic acid |
| between about 0.01% and | between about 0.01% and |
| about 0.5% | about 0.5% |
| between about 0.1% and | between about 0.1% and |
| about 0.4% | about 0.5% |
| about 0.125% | about 0.375% |
| about 0.225% | about 0.225% |
| about 0.25% | about 0.25% |
| about 0.375% | about 0.125% |
| about 0.3% | about 0.5% |

In a preferred embodiment of the present invention, the antimicrobial compositions comprises a compound of Formula I and one or more other acidulants. Such acidulants are typically strong acids, and are preferably mineral acids. Examples of such acidulants include phosphoric acid, phosphorous acid, sulfuric acid, hydrochloric acid, hydrobromic acid, and nitric acid. In one embodiment, the acidulant is phosphoric acid.

In a more preferred embodiment, the antimicrobial compositions comprise a compound of Formula I, one or more organic acids, as defined above, and one or more other acidulants as defined above.

Preferably, the pH of the feed is between about 3 and about 8. Even more preferably, the pH is between about 4 and about 7. Still more preferably, the pH is between about 4.5 and about 6.75. The pH may be measured by placing a known quantity of the feed and placing it in a known quantity of distilled water. The pH of the resulting solution, after sitting, may be measured by any standard means for measuring pH.

The following embodiments are particularly preferred for the addition of combinations of Alimet® and formic acid to feed (concentrations expressed in wt % of feed composition):

about 0.125% Alimet® and about 0.375% formic acid at pH from about 4.5 to about 6.75;

about 0.25% Alimet® and about 0.25% formic acid at pH from about 4.5 to about 6.75;

about 0.375% Alimet® and about 0.125% formic acid at pH from about 4.5 to about 6.75;

about 0.5% Alimet® at pH from about 4.5 to about 6.75;

about 0.3% Alimet® and about 0.5% formic acid at pH from about 4.5 to about 6.75.

In another preferred embodiment, the above-mentioned organic acid is a mixture of formic acid and propionic acid, wherein the formic acid comprises from about 95% to about 5% of the organic acid mixture and the propionic acid comprises from about 5% to about 95% of the organic mixture. Preferably, formic acid comprises from about 85% to about 15% of the organic acid mixture, and propionic acid comprises from about 15% to about 85% of the organic acid mixture. In another preferred embodiment, formic acid comprises from about 85% to about 65% of the organic acid mixture, and propionic acid comprises from about 15% to 35% of the organic acid mixture. In another preferred embodiment, formic acid comprises about 75% of the organic acid mixture, and propionic acid comprises about 25% of the organic acid mixture. This formic/propionic acid mixture can then be combined with the compound of Formula I according to the ratios described above.

In a preferred embodiment, the antimicrobial compositions comprise a compound of Formula I, preferably HMBA or a salt thereof, and a first organic acid, as defined herein. Preferably, the first organic acid is selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid. In another preferred embodiment, the antimicrobial compositions may further comprise one or more components selected from: a second organic acid, a third organic acid, and an acidulant. Preferably, the second organic acid and third organic acid are independently selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid. Preferably, the acidulant is selected from the group consisting of phosphoric acid, sulfuric acid, phosphorous acid, hydrochloric acid, hydrobromic acid, and nitric acid.

It has been discovered that certain antimicrobial compositions of the invention have an improved odor, when the compound of Formula I is Alimet®, compared to similar compositions without Alimet®. For example, blends comprising formic acid have a pungent odor. In such blends without Alimet®, this odor is more readily detectable than in the same blends containing Alimet®. Without being limited to a particular theory, it is believed that the Alimet® in the blends may lower the vapor pressure of the other organic acids in the blends. Alternatively, the Alimet® may mask the disagreeable odors.

In another preferred embodiment is provided a method for inhibiting bacteria in silage, said method comprising treating said silage with an anti-bacterial composition comprising a compound of Formula I. Preferably, the compound of Formula I is added to the silage at about 1 lb/ton to 80 lb/ton of fresh forage, more preferably at about 2 lb/ton to 50 lb/ton of fresh forage, more preferably about 3 lb/ton to 45 lb/ton of fresh forage, more preferably about 4 lb/ton to 40 lb/ton of fresh forage, more preferably about 5 lb/ton to 35 lb/ton of fresh forage, more preferably about 7 lb/ton to 30 lb/ton of fresh forage, more preferably about 9 lb/ton to 25 lb/ton of fresh forage, more preferably about 10 lb/ton to 20 lb/ton of fresh forage. Optionally, the compositions may further comprise an acidulant, as described herein.

In another preferred embodiment is provided a method for inhibiting bacteria in silage, said method comprising treating said silage with an anti-bacterial composition comprising a compound of Formula I and one or more organic acids as described above. Preferably, the compound of Formula I and other organic acid(s) are added to the silage at about 2 lb/ton to 125 lb/ton of fresh forage combined. In one embodiment, the compound of Formula I and other organic acid(s) are added to the silage at about 4 lb/ton to 100 lb/ton of fresh forage combined. In another embodiment, the compound of Formula I and other organic acid(s) are added to the silage at about 5 lb/ton to 90 lb/ton of fresh forage combined, more preferably about 7 lb/ton to 80 lb/ton of fresh forage combined, more preferably about 8 lb/ton to 70 lb/ton of fresh forage combined, more preferably about 9 lb/ton to 60 lb/ton of fresh forage combined, more preferably about 10 lb/ton to 55 lb/ton of fresh forage combined, more preferably about 12 lb/ton to 50 lb/ton of fresh forage combined, more preferably about 15 lb/ton to 30 lb/ton of fresh forage combined. Optionally, the compositions may further comprise an acidulant, as described herein.

In a preferred embodiment of the invention, the bacteria inhibited according to the methods of the present invention is from the family Enterobacteriaceae, Campylobacter or Lactobacillaceae. In another preferred embodiment, the bacteria is from the family Campylobacter or Lactobacillaceae. In another preferred embodiment, the bacteria is from the genus Lactobacillus or Campylobacter. In another preferred embodiment, the bacteria is L. plantarum or C. jejuni. In a particularly preferred embodiment, the bacteria is from the family Enterobacteriaceae. In an even more preferred embodiment, the bacteria is from the genus Salmonella or Escherichia. In a still more preferred embodiment, the bacteria is S. enteritidis or E. coli.

In yet another embodiment of the present invention, the above-mentioned compounds of Formula I may be used in the manufacture of a nutrient composition for inhibiting bacteria in animal feed. These nutrient compositions may further comprise one or more organic acids, as described above. Optionally, the compositions may further comprise an acidulant, as described herein.

In still yet another embodiment of the present invention, the above-mentioned compounds of Formula I may be used in a method for inhibiting bacteria in animal feed comprising computing the concentration in said feed of said compound of Formula I necessary to inhibit bacteria present in said animal feed, and applying said compound of Formula I to said feed in an amount sufficient to achieve said concentration.

In another embodiment of the present invention, the above-mentioned compounds of Formula I and above-mentioned organic acids may be used in a method of inhibiting bacteria in animal feed comprising computing the concentration in said feed of a compound of Formula I and another organic acid or mixture of organic acids necessary to inhibit bacteria present in said animal feed, and applying said compound of Formula I and said organic acid or mixture of organic acids to said feed in an amount sufficient to achieve said concentration.

In still yet another embodiment of the present invention, the above-mentioned compounds of Formula I may be used in a method of method for inhibiting mold in animal feed comprising directly or indirectly making information available for computing the concentration in said feed of a compound of Formula I necessary to inhibit bacteria present in said animal feed, and directly or indirectly making information available for applying said compound of Formula I to said feed in an amount sufficient to achieve said concentration.

In another embodiment of the present invention, the above-mentioned compounds of Formula I and above-mentioned organic acids may be used in a method of method for inhibiting mold in animal feed comprising directly or indirectly making information available for computing the concentration in said feed of said compound of Formula I and said organic acid or mixture of organic acids necessary to inhibit bacteria present in said animal feed, and directly or indirectly making information available for applying said compound of Formula I and said organic acid or mixture of organic acids to said feed in an amount sufficient to achieve said concentration.

In another embodiment of the present invention, the above-mentioned animal feeds may be heat-treated, either before or after administration of the above-mentioned compounds of Formula I and/or organic acids.

In yet another embodiment of the present invention, the above-mentioned compounds of Formula I may be used in a method of inhibiting bacteria in animal feed, the method comprising monitoring the concentration of methionine supplement in said feed composition, adding additional amounts of said methionine supplement as needed to achieve an anti-bacterially effective concentration of methionine supplement in said feed composition.

Treatment of the animal feed compositions with the compounds of Formula I and with the other organic acids disclosed herein may be done by mixing the compound of Formula I (and other organic acid, if present) with the other ingredients in the feed, such as the corn, soybean meal, meat meal premix, other feed supplements, etc., as the feed is being formulated. Alternatively, the compound of Formula I and optional other organic acid(s) may be applied to a pre-mixed or pre-pelleted feed. In either case, the compound of Formula I and optional organic acid(s) are preferably added as liquids, and uniformly disperse throughout the bulk of the feed composition when applied. When the compound of Formula I and another organic acid are both used in the methods of the present invention, preferably said compound of Formula I and said other organic acid or acids are mixed together before application to the animal feeds. This pre-mixed compound of Formula I/organic acid(s) blend can be applied to the animal feed ingredients during formulation of the feed compositions, or can be applied to pre-mixed or pre-pelleted feed.

In one embodiment of the present invention is presented a method of inhibiting mold in an animal feed composition, the method comprising applying a compound of Formula I to said feed composition, wherein said feed composition comprises corn and soy.

In another embodiment of the present invention, the methods of inhibiting mold in animal feed comprises treating said feed with an antifungally-effective amount of a compound of Formula I and one or more organic acids as described above.

In another preferred embodiment, the above-mentioned organic acid is a mixture of formic acid and propionic acid, wherein the formic acid comprises from about 95% to about 5% of the organic acid mixture and the propionic acid comprises from about 5% to about 95% of the organic mixture. Preferably, formic acid comprises from about 85% to about 15% of the organic acid mixture, and propionic acid comprises from about 15% to about 85% of the organic acid mixture. In another preferred embodiment, formic acid comprises from about 85% to about 65% of the organic acid mixture, and propionic acid comprises from about 15% to 35% of the organic acid mixture. This organic acid mixture can then be combined with the compound of Formula I according to the ratios described above.

In another embodiment of the present invention is presented a method of inhibiting mold in an animal feed composition, the method comprising applying a compound of Formula I and one or more organic acids to said feed composition, wherein said feed composition comprises corn and soy. In one embodiment, said organic acid is selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or combinations thereof. In another embodiment, the organic acid is formic acid, propionic acid, or combinations thereof.

In yet another embodiment of the present invention, the above-mentioned compounds of Formula I may be used in a method for delaying the formation of mold in an animal feed composition, the method comprising applying a compound of Formula I to said feed composition, wherein said feed composition comprises corn and soy.

In yet another embodiment of the present invention, the above-mentioned compounds of Formula I and above-mentioned organic acids may be used in a method for delaying the formation of mold in an animal feed composition, the method comprising applying a compound of Formula I and one or more organic acids to said feed composition, wherein said feed composition comprises corn and soy.

In yet another embodiment of the present invention, the above-mentioned compounds of Formula I may be used in a method of inhibiting the formation of mold in an animal feed composition, the method comprising applying a compound of Formula I to said feed composition, wherein said feed composition has a moisture content of abut 17% or less. Preferably, the moisture content is at least 0.01%. In another embodiment, the moisture content is at least 1%. In another embodiment, the moisture content is at least 5%. In another embodiment, the moisture content is at least 10%.

In yet another embodiment of the present invention, the above-mentioned compounds of Formula I and above-mentioned organic acids may be used in a method of inhibiting the formation of mold in an animal feed composition, the method comprising applying a compound of Formula I and one or more organic acids to said feed composition, wherein said feed composition has a moisture content of about 17% or less. Preferably, the moisture content is at least 0.01%. In another embodiment, the moisture content is at least 1%. In another embodiment, the moisture content is at least 5%. In another embodiment, the moisture content is at least 10%.

In yet another embodiment of the present invention is provided a method for inhibiting mold in silage, said method comprising treating said silage with an anti-fungal composition comprising a compound of Formula I. Preferably, the compound of Formula I is added to the silage at about 1 lb/ton to 40 lb/ton of fresh forage, more preferably about 5 lb/ton to 30 lb/ton of fresh forage, more preferably about 7 lb/ton to 25 lb/ton of fresh forage, more preferably about 10 lb/ton to 20 lb/ton of fresh forage.

In yet another embodiment of the present invention is provided a method for inhibiting mold in silage, said method comprising treating said silage with an anti-fungal composition comprising a compound of Formula I and one or more organic acids. Preferably, the compound of Formula I and other organic acid(s) are added to the silage at about 5 lb/ton to 50 lb/ton of fresh forage combined, more preferably about 8 lb/ton to 40 lb/ton of fresh forage combined, more preferably about 10 lb/ton to 30 lb/ton of fresh forage combined, more preferably about 15 lb/ton to 25 lb/ton of fresh forage combined.

In yet another embodiment of the present invention, the above-mentioned compounds of Formula I may be used in a method for inhibiting the growth of mold in an animal feed composition, the method comprising computing the concentration in said feed of a compound of Formula I necessary to inhibit the growth of mold in said feed composition; and applying said compound of Formula I to said feed composition in said concentration.

In yet another embodiment of the present invention, the above-mentioned compounds of Formula I and organic acids may be used in a method for inhibiting the growth of mold in an animal feed composition, the method comprising computing the concentration in said feed of a compound of Formula I and another organic acid or mixture of organic acids necessary to inhibit the growth of mold in said feed composition; and applying said compound of Formula I and said organic acid or mixture of organic acids to said feed composition in said concentration.

In yet another embodiment of the present invention, the above-mentioned compounds of Formula I may be used in a method for inhibiting the growth of mold in animal feed comprising computing the concentration in said feed of a compound of Formula I necessary to inhibit the growth of mold in said animal feed; and applying said compound of Formula I to said feed in said concentration.

In yet another embodiment of the present invention, the above-mentioned compounds of Formula I and organic acids may be used in a method for inhibiting the growth of mold in animal feed comprising computing the concentration in said feed of a compound of Formula I and another organic acid or mixture of organic acids necessary to inhibit the growth of mold in said animal feed; and applying said compound of Formula I and said organic acid or mixture of organic acids to said feed in said concentration.

In yet another embodiment of the present invention, the above-mentioned compounds of Formula I may be used in a method for improving the mold resistance of an animal feed composition, the method comprising discontinuing the use of DL-methionine as a feed supplement; computing the concentration in said feed of a compound of Formula I necessary to inhibit the growth of mold in said animal feed; and applying said compound of Formula I to said feed in said concentration.

In yet another embodiment of the present invention, the above-mentioned compounds of Formula I and said organic acids may be used in a method for improving the mold resistance of an animal feed composition, the method comprising discontinuing the use of DL-methionine as a feed supplement; computing the concentration in said feed of a compound of Formula I and another organic acid or mixture of organic acids necessary to inhibit the growth of mold in said animal feed; and applying said compound of Formula I and said organic acid or mixture of organic acids to said feed in said concentration.

In yet another embodiment of the present invention, the above-mentioned compounds of Formula I may be used in a method for inhibiting mold in animal feed directly or indirectly making information available for computing the concentration in said feed of said compound of Formula I necessary to inhibit mold present in said animal feed; and directly or indirectly making information available for applying said compound of Formula I to said feed in said concentration.

In yet another embodiment of the present invention, the above-mentioned compounds of Formula I and organic acids may be used in a method for inhibiting mold in animal feed directly or indirectly making information available for computing the concentration in said feed of said compound of Formula I and the concentration of said organic acid or mixture of organic acids necessary to inhibit mold present in said animal feed; and directly or indirectly making information available for applying said compound of Formula I and said organic acid or mixture of organic acids to said feed in said concentration.

In yet another embodiment of the present invention, the above-mentioned compounds of Formula I may be used in the manufacture of a nutrient composition for inhibiting mold in animal feed by treating said feed with said nutrient composition in an anti-mold effective amount. In another embodiment, the nutrient composition may also comprise one or more of the above-mentioned organic acids, or a mixture thereof.

In yet another embodiment of the present invention, the above-mentioned compounds of Formula I may be used in a method of inhibiting mold in an animal feed composition, the method comprising monitoring the concentration of methionine supplement in said feed composition, adding additional amounts of said methionine supplement as needed to achieve an anti-mold effective concentration of methionine supplement in said feed composition.

In yet another embodiment of the present invention, the above-mentioned compounds of Formula I may be used in a method of enhancing the palatability of animal food, the method comprising treating the food with a compound of Formula I in an amount sufficient to give a concentration of the compound of Formula I in the food of between about 0.01 wt. % and about 0.5 wt. %. Preferably, the food is food for canines, felines, or aquaculture. For dogs, the concentration of the compound of Formula I in the food is preferably between about 0.05% and about 0.15%; for cats, it is preferably between about 0.20% and 0.30%. For both dogs and cats, the compound of Formula I is preferably HMBA or DLM.

In a preferred embodiment, the compositions or combinations described herein comprise HMBA, or a salt, ester or amide thereof; and a first organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid. Even more preferably, the first organic acid is selected from the group consisting of formic acid, propionic acid, butyric acid, and lactic acid.

In various preferred embodiments, the compositions or combinations further comprise an acidulant selected from the group consisting of mineral acids, preferably selected from the group consisting of phosphoric acid, sulfuric acid, phosphorous acid, hydrochloric acid, hydrobromic acid, and nitric acid; a second organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid. In even more preferred embodiments, the first organic acid and second organic acid are independently selected from the group consisting of formic acid, propionic acid, butyric acid, and lactic acid; and/or a third organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid.

In a still more preferred embodiment, the compositions and combinations described herein comprise HMBA, formic acid, propionic acid, and phosphoric acid. Preferably, the content of HMBA is from about 5% to about 20% of the sum of the WRA, formic acid, propionic acid, and phosphoric acid content; the content of the formic acid is from about 65% to about 85% of said sum; the content of the propionic acid is from about 1% to about 15% of said sum; and the content of the phosphoric acid is from about 5% to about 20% of said sum. Even more preferably, the content of HMBA is about 10% of said sum; the content of the formic acid is about 75% of said sum; the content of the propionic acid is about 5% of said sum; and the content of the phosphoric acid is about 10% of said sum. Alternatively, the content of HMBA is from about 20% to about 40% of said sum, the content of the formic acid is from about 45% to about 65% of said sum, the content of the propionic acid is from about 1% to about 20% of said sum, and the content of the phosphoric acid is from about 1% to about 15% of said sum; even more preferably, the content of HMBA is about 30% of said sum, the content of the formic acid is about 55% of said sum, the content of the propionic acid is about 10% of said sum, and the content of the phosphoric acid is about 5% of said sum.

In another more preferred embodiment, the compositions and combinations described herein comprise HMBA, butyric acid, lactic acid, and phosphoric acid. Preferably, the content of HMBA is from about 20% to about 40% of the sum of the HMBA, butyric acid, lactic acid, and phosphoric acid content; the content of the butyric acid is from about 10% to about 30% of said sum; the content of the lactic acid is from about 10% to about 30% of said sum; and the content of the phosphoric acid is from about 20% to about 40% of said sum. Even more preferably, the content of HMBA is about 30% of said sum; the content of the butyric acid is about 20% of said sum; the content of the lactic acid is about 20% of said sum; and the content of the phosphoric acid is about 30% of said sum. Alternatively, the content of HMBA is from about 20% to about 40% of said sum of the 2-hydroxy-4-(methylthio)butanoic acid, butyric acid, lactic acid, and phosphoric acid content, the content of the butyric acid is from about 5% to about 25% of said sum, the content of the lactic acid is from about 10% to about 30% of said sum, and the content of the phosphoric acid is from about 25% to about 45% of said sum; more preferably, the content of HMBA is about 30% of said sum, the content of the butyric acid is about 15% of said sum, the content of the lactic acid is about 20% of said sum, and the content of the phosphoric acid is about 35% of said sum.

In yet another more preferred embodiment, the compositions and combinations described herein comprise HMBA, butyric acid, formic acid, lactic acid, and phosphoric acid. Preferably, the content of HMBA is from about 10% to about 30% of the sum of the HMBA, butyric acid, formic acid, lactic acid, and phosphoric acid content; the content of the butyric acid is from about 2% to about 22% of said sum; the content of the formic acid is from about 20% to about 40% of said sum; the content of the lactic acid is from about 8% to about 28% of said sum; and the content of the phosphoric acid is from about 10% to about 30% of said sum. Even more preferably, the content of HMBA is about 20% of said sum; the content of the butyric acid is about 12% of said sum; the content of the formic acid is about 30% of said sum; the content of the lactic acid is about 18% of said sum; and the content of the phosphoric acid is about 20% of said sum.

In yet another more preferred embodiment, the compositions and combinations described herein comprise HMBA, butyric acid, lactic acid, propionic acid, and phosphoric acid. Preferably, the content of HMBA is from about 10% to about 30% of the sum of the HMBA, butyric acid, lactic acid, propionic acid, and phosphoric acid content; the content of the butyric acid is from about 2% to about 22% of said sum; the content of the lactic acid is from about 8% to about 28% of said sum; the content of the propionic acid is from about 20% to about 40% of said sum; and the content of the phosphoric acid is from about 10% to about 30% of said sum. Even more preferably, the content of HMBA is about 20% of said sum; the content of the butyric acid is about 12% of said sum; the content of the lactic acid is about 18% of said sum; the content of the propionic acid is about 30% of said sum; and the content of the phosphoric acid is about 20% of said sum.

In yet another more preferred embodiment, the compositions and combinations described herein comprise HMBA, butyric acid, formic acid, propionic acid, and phosphoric acid. Preferably, the content of HMBA is from about 1% to about 20% of the sum of the HMBA, butyric acid, formic acid, propionic acid, and phosphoric acid content; the content of the butyric acid is from about 1% to about 15% of said sum; the content of the formic acid is from about 65% to about 85% of said sum; the content of the propionic acid is from about 1% to about 15% of said sum; and the content of the phosphoric acid is from about 1% to about 15% of said sum. Even more preferably, the content of HMBA is about 10% of said sum; the content of the butyric acid is about 5% of said sum; the content of the formic acid is about 75% of said sum; the content of the propionic acid is about 5% of said sum; and the content of the phosphoric acid is about 5% of said sum.

In yet another more preferred embodiment, the compositions and combinations described herein comprise HMBA, formic acid, and propionic acid. Preferably, the content of HMBA is from about 20% to about 40% of the sum of the HMBA, formic acid, and propionic acid content; the content of the formic acid is from about 40% to about 60% of said sum; and the content of the propionic acid is from about 10% to about 30% of said sum. Even more preferably, the content of HMBA is about 30% of said sum; the content of the formic acid is about 50% of said sum; and the content of the propionic acid is about 20% of said sum.

In yet another more preferred embodiment, compositions and combinations described herein comprise HMBA and phosphoric acid. Preferably, the content of the HMBA is from about 5% to about 50% of the sum of the HMBA and phosphoric acid content. In various more preferred embodiments, the content of the HMBA is about 5%, or about 10%, or about 15%, or about 20%, or about 25%, or about 30%, or about 35%, or about 40%, or about 45%, or about 50% of said sum.

In another embodiment of the present invention is provided an animal feed composition comprising a compound of Formula I as described herein, and an acidulant as described herein.

In another embodiment of the present invention is provided a method of inhibiting or killing microbes in a subject, comprising treating said subject with a composition or combination as described herein. In a preferred embodiment, the subject to be treated is water or food, preferably selected from the group consisting of human food, livestock food, pet food, or aquaculture food.

Animals for which the food, food ingredients and/or feed compositions described herein may be provided include humans, ruminants such as dairy cows, lactating dairy cows, dairy calves, beef cattle, sheep, and goats; aquaculture such as fish and crustaceans (including, but not limited to, salmon, shrimp, carp, tilapia and shell fish; livestock such as swine and horses; poultry such as chickens, turkeys, and hatchlings thereof; and companion animals such as dogs and cats.

The exact formulation of the above-mentioned animal feed composition is not critical to the present invention. Feed ingredients are selected according to the nutrient requirements of the particular animal for which the feed is intended; these requirements depend, inter alia, upon the age and stage of development of the animal, the sex of the animal, and other factors. Feed ingredients may be grouped into eight classes on the basis of their composition and their use in formulating diets: dry forages and roughages; pasture, range plants and forages fed fresh; silages; energy feeds; protein supplements; mineral supplements; vitamin supplements; and additives. See National Research Council (U.S.) Subcommittee on Feed Composition, United States-Canadian Tables of Feed Composition, 3d rev., National Academy Press, pp. 2, 145 (1982). These classes are, to a certain extent, arbitrary, as some feed ingredients could be classified in more than one class. Typically, a feed formulation will also depend upon the costs associated with each ingredient, with the least-expensive composition of ingredients which gives the needed nutrients being the preferred formulation.

Silage is a forage product that is produced from the harvest, storage and fermentation of green forage crops such as corn and grain sorghum plants. These crops are chopped, stems and all, before the grain is ready for harvest. The plant material is stored in silos, storage bags, bunkers or covered piles causing the material to ferment, thereby lowering the pH and preserving the plant material until it can be fed. The ensiled forage is subject to changes in pH, temperature, and oxygen levels.

As noted above, feed formulations depend in part upon the age and stage of development of the animal to be fed. Leeson and Summers (Nutrition of the Chicken, 4th ed., pp. 502-510, University Books (2001)) describe several representative poultry diets for pullets, layers, broilers and broiler breeders. For example, most chicken diets contain energy concentrates such as corn, oats, wheat, barley, or sorghum; protein sources such as soybean meal, other oilseed meals (e.g., peanut, sesame, safflower, sunflower, etc.), cottonseed meal, animal protein sources (meat and bone meal, dried whey, fish meal, etc.), grain legumes (e.g., dry beans, field peas, etc.), and alfalfa;

and vitamin and mineral supplements, if necessary (for instance, meat and bone meal is high in calcium and phosphorous, and thus these minerals do not need to be supplemented in a feed ration containing meat and bone meal). The relative amounts of the different ingredients in poultry feed depends in part upon the production stage of the bird. Starter rations are higher in protein, while grower and finisher feeds can be lower in protein since older birds require less protein. Model diets for swine and other animals are also available, and may be modified according to the particular needs of the animal(s) to be fed.

The term “inhibit” when used herein in phrases such as “inhibiting bacteria” means any one or more of (a) killing bacteria or mold; (b) any decrease in growth of the bacteria or mold, which may be measured in terms of colony counts; (c) any decrease in the concentration of bacteria or mold; or (d) the inability of bacteria or mold to grow on a particular selection medium. Each of these may be determined, for instance, by comparing the bacterial or fungal colony counts or concentration of bacteria or mold present in the absence of the application of the methods of the present invention with the bacterial or fungal colony counts or concentration of bacteria or mold after application of the methods of the present invention. Application of suitable bactericides or fungicides will show a ten-fold difference in colony counts.

Certain methods of the present invention call for computing, for instance, the concentration, of a compound of Formula I in an animal feed, necessary to inhibit bacteria or mold present in an animal feed, or the concentration of a compound of Formula I and another organic acid or acids necessary to inhibit bacteria or mold present in an animal feed. Provided herein are Examples 1-24, which illustrate amounts of compound of Formula I and/or other organic acids that are sufficient to inhibit bacteria or mold. Also provided hereinabove and hereinbelow are acceptable ranges of amounts of compound of Formula I and/or other organic acids, and ratios between the two, which are suitable for use the methods of the present invention. Other suitable concentrations, ranges and ratios can be determined as needed.

Treatment of the animal feed compositions with the compounds of Formula I and optionally with the other organic acids disclosed herein, or with the compounds of Formula I and optionally with other organic acid(s), may be done by mixing the compound of Formula I (and other organic acid, if present) with the other ingredients in the feed, such as the corn, soybean meal, other feed supplements, etc., as the feed is being formulated. Alternatively, the compound of Formula I and optional other organic acid(s) may be applied to a pre-mixed or pre-pelleted feed. In either case, the compound of Formula I and optional organic acid(s) are preferably added as liquids, and uniformly disperse throughout the bulk of the feed composition when applied. When the compound of Formula I and another organic acid are both used in the methods of the present invention, preferably said compound of Formula I and said other organic acid or acids are mixed together before application to the animal feeds. This pre-mixed compound of Formula I/organic acid(s) blend can be applied to the animal feed ingredients during formulation of the feed compositions, or can be applied to pre-mixed or pre-pelleted feed.

The term “cfu” stands for colony forming units.

The following examples illustrate the invention.

### EXAMPLE 1

The effects of increasing quantities of formic acid and/or Alimet® on the colony counts of four bacteria (E. coli, S. enteritidis, L. plantarum and C. jejuni) were studied. Varying amounts of formic acid or Alimet® were added individually to cultures of these bacteria at pH 4.5 or 6.75 and the cultures were incubated for a length of time, whereupon colony counts were performed.

The S. enteritidis culture for the in vitro study contained a mixture of S. enteritidis ID-Lelystad (nalidixic acid resistant strain) and S. enteritidis (97.07773 RIVM, isolated from poultry). The E. coli culture contained a mixture of E. coli 0149K91K88 (VA2000-08915, pig pathogen) and E. coli ATCC 25922. The L. plantarum culture studied was L. plantarum Bd 99.00553. The C. jejuni culture studied was C. jejuni C356, ex. ID-Lelystad.

S. enteritidis and E. coli from fresh overnight cultures in Brain Heart Infusion broth were incubated aerobically in phosphate buffered (0.11 M) salt solution (8.5 g/L NaCl) with peptone (1 g/L), except for S. enteritidis at pH 4.5. For this culture, medium 5 was used as the broth, and the culture was incubated aerobically for 4 hours at 37° C. Colony counts were performed according to standard operating procedures.

The fresh overnight culture of L. plantarum in brain heart infusion broth were used to inoculate medium 5. The test tubes were incubated under reduced oxygen atmosphere for 6 hours at 37° C. Colony counts were performed.

C. jejuni grown on Campylobacter blood-free selective agar was used for inoculation. Preston broth was incubated under reduced oxygen atmosphere for 6 hours at 37° C. Colony counts were performed.

Formic acid and Alimet® were added to the bacterial cultures in concentrations of 0.108 g/L, 0.30 g/L and 0.83 g/L. These dosages were chosen based on commercial use of Alimet® and an approximate 10-fold dilution in the proximal digestive tract.

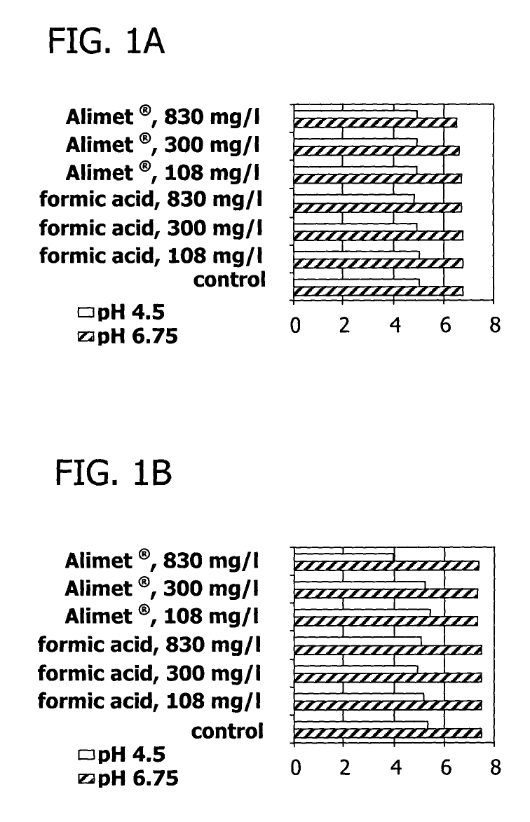

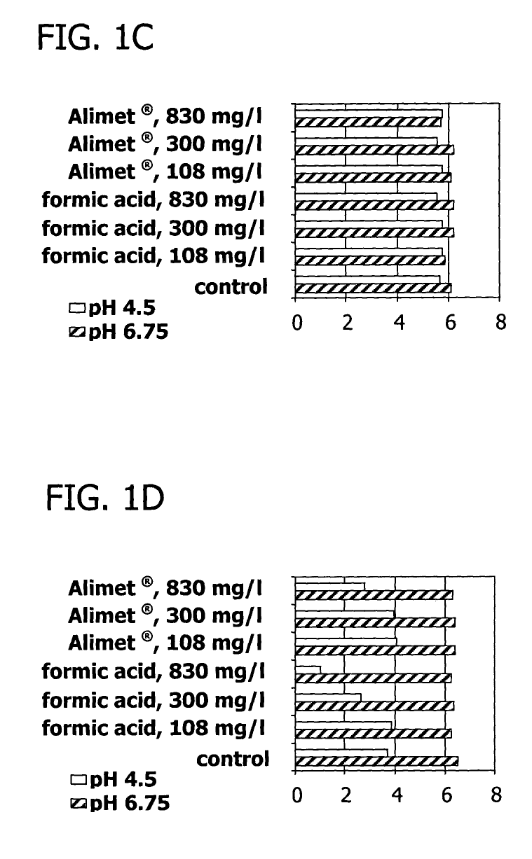

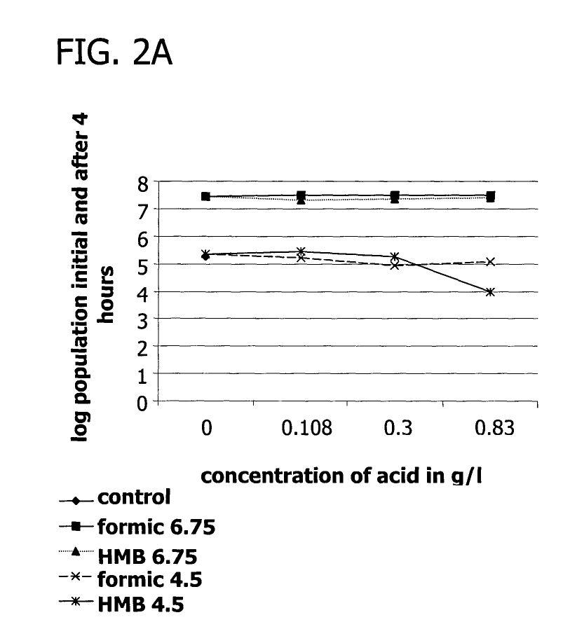

A summary of the results obtained with formic acid and Alimet® on S. enteritidis and E. coli is given in Tables 1 and 2, and on L. plantarum and C. jejuni in Tables 3 and 4; the results are illustrated in FIGS. 1-4.

TABLE 1
Effect of formic acid and Alimet ® on population of
S. enteritidis after 4 hours at pH 4.5 and 6.75
initial colony count: 5.23 log cfu/mL

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 5.03 | −0.20 | 6.62 | 1.42 |
| 0.108 | — | 4.92 | −0.31 | 6.71 | 1.51 |
| 0.30 | — | 4.96 | −0.27 | 6.63 | 1.43 |
| 0.83 | — | 4.93 | −0.30 | 6.53 | 1.33 |
| — | 0.108 | 5.04 | −0.19 | 6.79 | 1.59 |
| — | 0.30 | 4.96 | −0.27 | 6.77 | 1.57 |
| — | 0.83 | 4.86 | −0.38 | 6.72 | 1.52 |

TABLE 2
Effect of formic acid and Alimet ® on population of
E. coli after 4 hours at pH 4.5 and 6.75
initial colony count: 5.24 log cfu/mL

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 5.36 | 0.12 | 7.47 | 2.23 |
| 0.108 | — | 5.45 | 0.21 | 7.33 | 2.09 |
| 0.30 | — | 5.25 | 0.01 | 7.36 | 2.12 |
| 0.83 | — | 3.96 | −1.28 | 7.39 | 2.15 |
| — | 0.108 | 5.19 | −0.05 | 7.48 | 2.24 |
| — | 0.30 | 4.96 | −0.28 | 7.50 | 2.26 |
| — | 0.83 | 5.08 | −0.16 | 7.49 | 2.25 |

TABLE 3
Effect of formic acid and Alimet ® on population of
L. plantarum after 6 hours at pH 4.5 and 6.75
initial colony count: 5.04 log cfu/mL
Δ log = logₛₐₘₚₗₑ − logᵢₙᵢₜᵢₐₗ

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 5.67 | 0.63 | 6.10 | 1.06 |
| 0.108 | — | 5.67 | 0.63 | 6.09 | 1.05 |
| 0.30 | — | 5.57 | 0.53 | 6.20 | 1.16 |
| 0.83 | — | 5.74 | 0.70 | 5.70 | 0.66 |
| — | 0.108 | 5.75 | 0.71 | 5.88 | 0.84 |
| — | 0.30 | 5.74 | 0.70 | 6.23 | 1.19 |
| — | 0.83 | 5.56 | 0.52 | 6.19 | 1.15 |

TABLE 4
Effect of formic acid and Alimet ® on population of
C. jejuni after 6 hours at pH 4.5 and 6.75
initial colony count: 5.23 log cfu/mL

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 3.70 | −1.53 | 6.54 | 1.31 |
| 0.108 | — | 0.108 | 4.07 | −1.16 | 6.44 |
| 0.30 | — | 0.30 | 3.95 | −1.28 | 6.40 |
| 0.83 | — | 0.83 | 2.80 | −2.43 | 6.34 |
| — | 0.108 | 3.86 | −1.37 | 6.27 | 1.04 |
| — | 0.30 | 2.63 | −2.60 | 6.38 | 1.15 |
| — | 0.83 | <1.30 | <−3.93 | 6.25 | 1.02 |

S. enteritidis: Prior to inoculation, the S. enteritidis cultures had a colony count of 5.03 log cfu/mL at pH 4.5, and of 6.62 at pH 6.75. The results obtained at both pH values were similar for Alimet® and formic acid: at pH 4.5, neither had a significant effect on inhibiting the growth of S. enteritidis; at pH 6.75, no inhibition of S. enteritidis was observed.

E. coli: Prior to inoculation, at pH 4.5, the E. coli cultures had a colony count of 5.36 log cfu/mL; at pH 6.75, it was 7.47. 0.83 g/L Alimet® gave approximately a 1.3 log reduction of E. coli growth at pH 4.5, compared to the approximately 0.1 log reduction by the same concentration of formic acid at pH 4.5. Lower concentrations of both Alimet® and formic acid showed little or no inhibition. Neither Alimet® nor formic acid inhibited E. coli at pH 6.75.

L. plantarum: Prior to inoculation, at pH 4.5, the L. plantarum cultures had a colony count of 5.67 log cfu/mL; at pH 6.75, it was 6.10. Neither Alimet® nor formic acid inhibited L. plantarum at either pH studied.

C. jejuni: Prior to inoculation, at pH 4.5, the C. jejuni cultures had a colony count of 3.70 log cfu/mL; at pH 6.75, it was 6.54. All doses of Alimet® inhibited C. jejuni at pH 4.5. 0.83 g/L of Alimet® gave approximately a 2.4 log reduction of C. jejuni growth at this pH. Lower dosages of Alimet® (0.108 g/L and 0.30 g/L) gave approximately 1.1 and 1.2 log reductions, respectively. Formic acid demonstrated comparable inhibition. No antibacterial activity was shown against C. jejuni at pH 6.75 for any Alimet® or formic acid concentration studied.

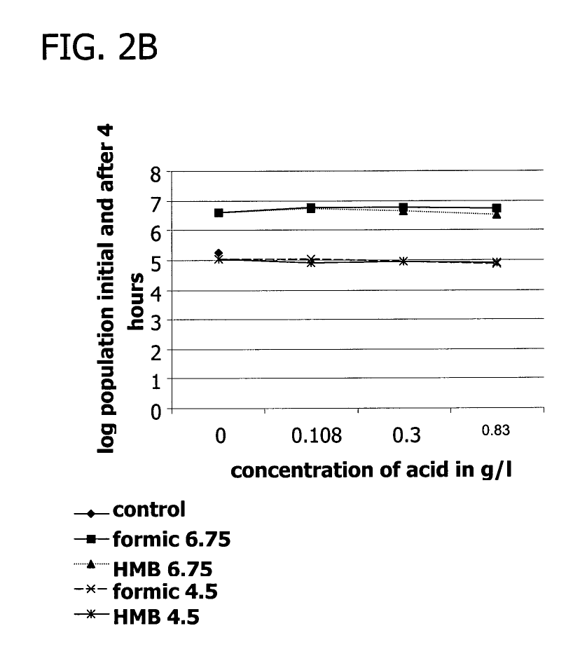

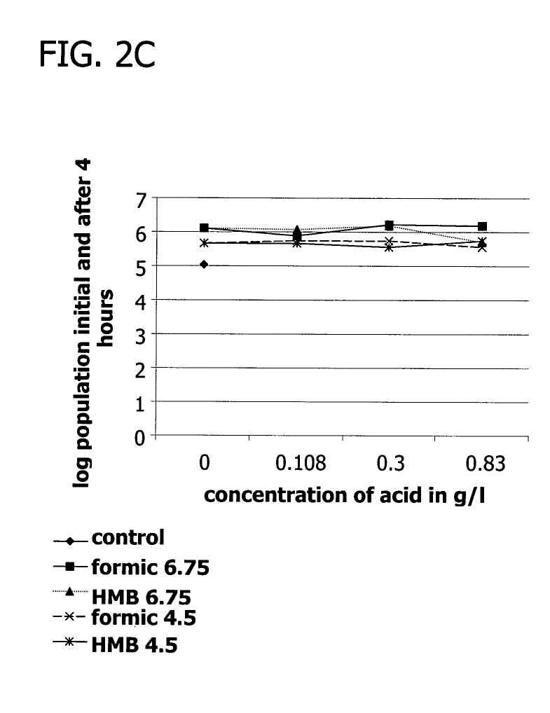

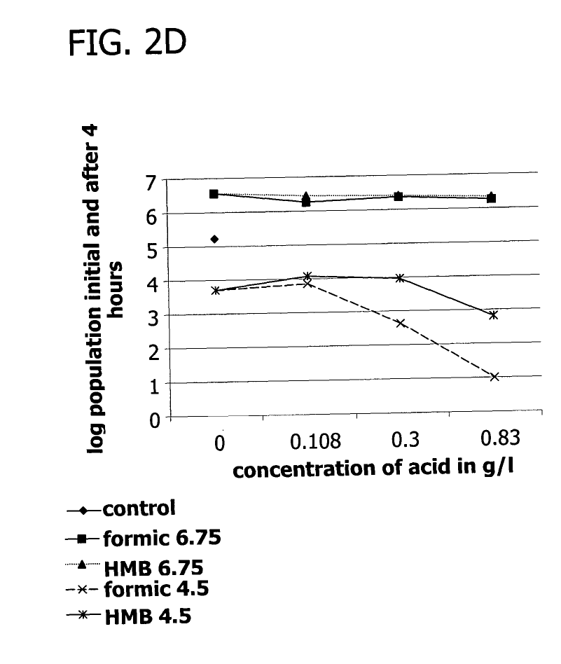

These results are demonstrated graphically in FIGS. 1A, 1B, 1C, and 1D. FIGS. 2A, 2B, 2C, and 2D demonstrate the pH dependent effects of formic acid and Alimet®. None of the four bacteria studied were inhibited by either formic acid or Alimet® at pH of 6.75; in fact, at this pH, the colony forming unit count of each bacteria increased, with the E. coli count increasing the most, and L. plantarum increasing the least.

### EXAMPLE 2

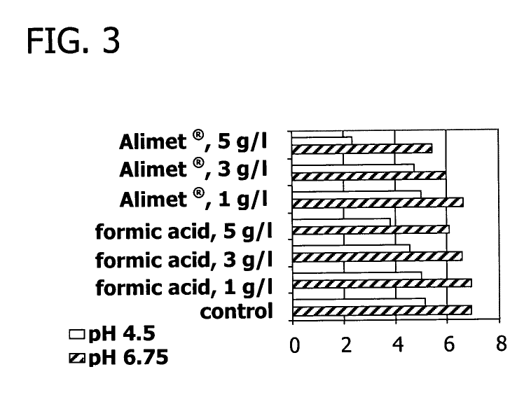

The effect of higher dosages of Alimet® and formic acid on the colony count of S. enteritidis cultures was studied, following the procedure described in Example 1. The results obtained are given in Table 5 and illustrated in FIG. 3.

TABLE 5
Effect of formic acid and Alimet ® on population of
S. enteritidis after 4 hours at pH 4.5 and 6.75
initial colony count: 5.23 log cfu/mL

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 5.15 | −0.09 | 6.92 | 1.69 |
| 1 | — | 5.02 | −0.21 | 6.61 | 1.38 |
| 3 | — | 4.76 | −0.48 | 5.97 | 0.74 |
| 5 | — | 2.37 | −2.86 | 5.43 | 0.20 |
| — | 1 | 5.01 | −0.22 | 6.92 | 1.69 |
| — | 3 | 4.55 | −0.68 | 6.58 | 1.35 |
| — | 5 | 3.83 | −1.41 | 6.10 | 0.87 |

Prior to inoculation, the S. enteritidis cultures had a log cfu/mL of 5.15 at pH 4.5, and of 6.92 at pH 6.75. At pH 6.75, the addition of 5 g/L formic acid or 3 g/L Alimet® gave approximately a 1 log cfu/mL growth inhibition. An addition of 5 g/L Alimet® stops the growth of S. enteritidis. At pH 4.5, 5 g/L Alimet® reduces the growth of S. enteritidis by approximately 2.8 log cfu/mL. Lower concentrations of Alimet® gives a smaller effect. Formic acid at 5 g/L reduces the growth of S. enteritidis by approximately 1.3 log cfu/mL. Thus, at the dose ranges studied, the antibacterial effect of Alimet® against S. enteritidis is greater than that of formic acid. These results are demonstrated graphically in FIG. 3.

### EXAMPLE 3

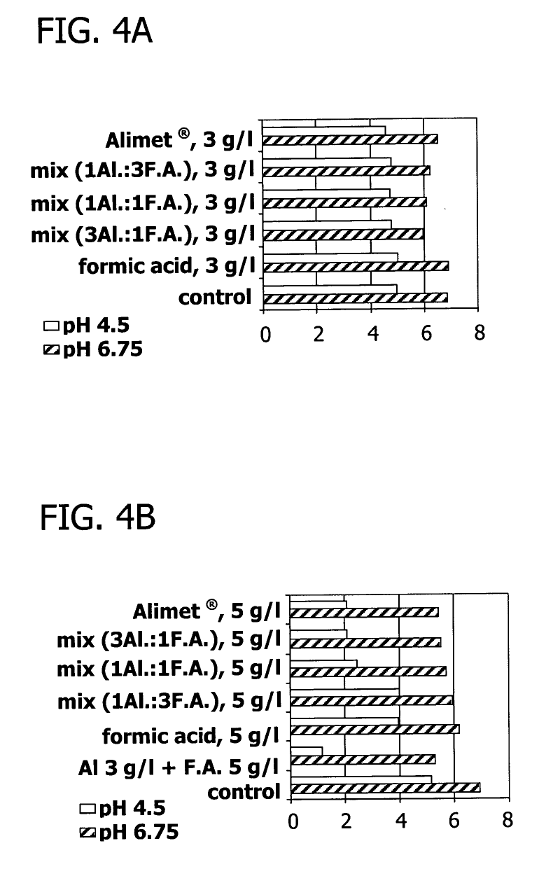

Combinations of Alimet® and formic acid were studied, following the procedure described in Example 1. The results obtained are given in Table 6 and illustrated in FIGS. 4A and 4B.

TABLE 6
Effect of formic acid and Alimet ® on population of
S. enteritidis after 4 hours at pH 4.5 and 6.75
initial colony count: 5.15 log cfu/mL)

| Acid (g/L) | | pH = 4.5 | | pH = 6.75 | |
|---|---|---|---|---|---|
| Alimet ® | Formic | log cfu/mL | Δ log | log cfu/mL | Δ log |
| — | — | 4.99 | −0.17 | 6.87 | 1.80 |
| 3 | — | 4.76 | −0.39 | 5.89 | 0.82 |
| 5 | — | 2.07 | −3.08 | 5.45 | 0.38 |
| — | 3 | 4.57 | −0.58 | 6.51 | 1.44 |
| — | 5 | 3.94 | −1.21 | 6.19 | 1.12 |
| 0.75 | 2.25 | 4.78 | −0.37 | 6.27 | 1.20 |
| 1.25 | 3.75 | 4.01 | −1.14 | 5.94 | 0.88 |
| 1.50 | 1.50 | 4.73 | −0.42 | 6.11 | 1.04 |
| 2.25 | 0.75 | 4.78 | −0.37 | 5.97 | 0.90 |
| 2.50 | 2.50 | 2.48 | −2.67 | 5.74 | 0.67 |
| 3 | 5 | 1.15 | −4.00 | 5.31 | 0.24 |
| 3.75 | 1.25 | 2.11 | −3.04 | 5.54 | 0.47 |

Combinations of Alimet® and formic acid having a combined concentration of 5 g/L inhibit growth of S. enteritidis to a greater extent than do combinations having a combined concentration of 3 g/L. Three 5 g/L combinations were prepared, having Alimet®-to-formic acid ratios of 1:3, 1:1, and 3:1.

At pH 4.5, treatment with 3 g/L of Alimet® alone gave an approximately 0.4 log cfu/mL reduction in S. enteritidis growth. Treatment at that pH with 5 g/L of formic acid gave an approximately 1.2 log cfu/mL reduction. Remarkably, treatment with a combination of 3 g/L Alimet® and 5 g/L formic acid gave a reduction of 4 log cfu/mL, which was higher than expected given the individual results with Alimet® and formic acid at those levels. The results obtained suggest that at pH 4.5, combinations of 2.5 g/L Alimet® and 2.5 g/L formic acid, and with 3 g/L Alimet® and 5 g/L formic acid may have a synergistic effect. The latter combination gives the best results of all tested combinations: at pH 4.5, this combination gives 4 log (almost complete) reduction of S. enteritidis.

### EXAMPLE 4

The effects of blends of organic acids (butyric, citric, formic, lactic, and propionic) and Alimet® on the colony counts of E. coli (ATCC 25922) grown in trypticase soy broth at 35° C. according to the manufacturer's instructions were studied. Blends of organic acid:Alimet® of 2:1 and 5:1 were studied, at a total concentration (organic acid+Alimet®) of 6 g/L.

The pH of the solutions were originally adjusted to pH 5 by addition of HCl and/or NaOH as needed. Activated E. coli culture solutions were transferred to fresh soy broth twice at 24-hour intervals prior to before addition of the organic acid:Alimet® blend. E. coli culture solutions were centrifuged; pellet produced was re-suspended with Butterfield buffer, and the resulting solutions were diluted to approximately 10⁷ CFU E. coli/mL.

Bottles were inoculated with 100 μL of prepared bacterial suspension and an organic acid:Alimet® blend. Samples were taken after five and 24 hours of incubation, serially diluted and spread-plated on trypticase soy agar, and incubated at 35° C. for 24 hours. Populations of E. coli are reported in Table 7 below.

TABLE 7
Effect of Alimet ®/acid blends on E. coli populations
in trypticase soy broth initial colony count: 4.97 log cfu/mL

| | | |
|---|---|---|
| Acid:Alimet ® | original | log cfu/mL |

| Acid | ratio | pH | t = 4 h | t = 24 h |
|---|---|---|---|---|
| Control | — | — | 7.18 | 9.22 |
| HCl | — | — | 7.85 | 8.34 |
| lactic | 5:1 | ca. 4.2 | 4.68 | 3.98 |
| | 2:1 | 4.1 | 4.87 | 4.45 |
| formic | 5:1 | 3.1 | 4.95 | <1 |
| | 2:1 | 3.56 | 4.95 | 1.00 |
| citric | 5:1 | 4.75 | 6.38 | 8.59 |
| | 2:1 | 4.59 | 5.90 | 8.66 |
| butyric | 5:1 | 4.62 | 4.77 | 3.70 |
| | 2:1 | 4.6 | 4.85 | 3.80 |
| propionic | 5:1 | 4.54 | 4.79 | 4.57 |
| | 2:1 | 4.53 | 4.83 | 4.53 |

Blends with formic acid were the most effective among the tested blends to control E. coli at both 5:1 and 2:1 blends of formic acid:Alimet®. Upon prolonged exposure (after 5 hours), both ratio give nearly complete reduction of E. coli. Blends of lactic, butyric and propionic acids with Alimet® suppressed the growth of E. coli, but did not reduce the bacterial population in 24 hours.

### EXAMPLE 5

The effects of hydrochloric acid, formic acid, lactic acid, or Alimet® on the colony counts of E. coli were studied. Amounts of formic acid, lactic acid, or Alimet® were added to cultures of E. coli, grown in a soy broth, at pH 4 or 7.3. The cultures were incubated, and colony counts performed at increasing times.

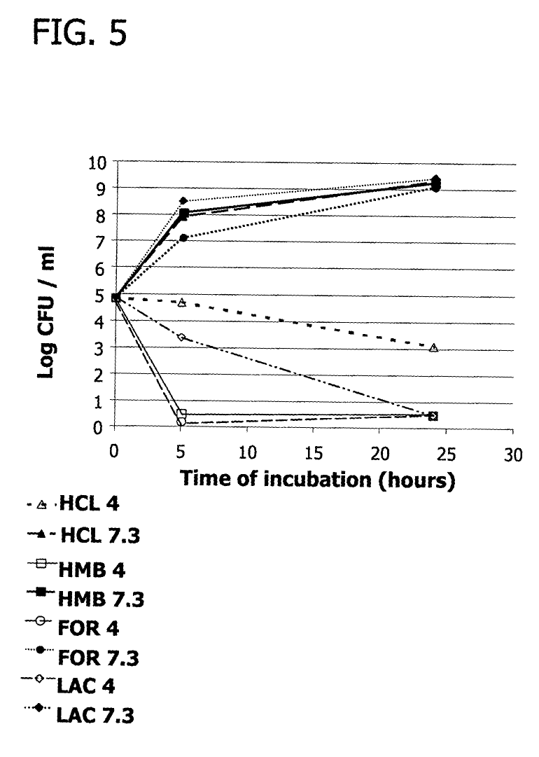

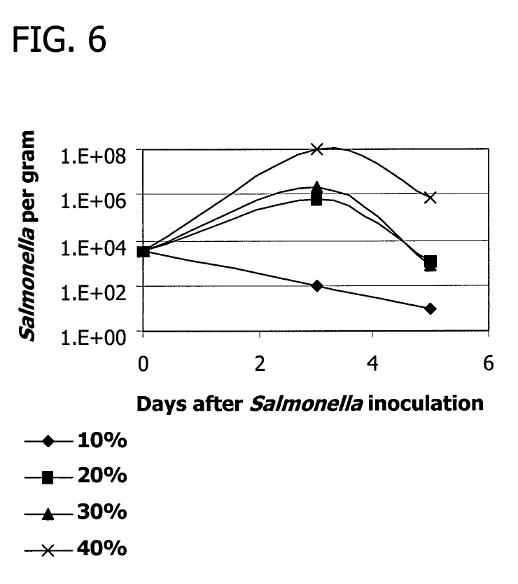

The results are illustrated in FIG. 5.

Formic and lactic acid, and Alimet®, decreased the colony counts of E. coli better than hydrochloric acid. As in Example 1, Alimet® showed a better reduction of E. coli colony counts as compared to formic acids, and showed comparable reduction compared to lactic acid.

### EXAMPLE 6

The effect of Alimet® on Salmonella in a meat meal premix was studied according to the protocol set forth by Smyser and Snoeyenbos (Poultry Sci. 58 (1979) 50-54). Meat meal premix (Papillon Ag Products, Inc., Easton, Md.) containing approximately 77% crude protein was used in the assays. Ten grams of premix test sample for each concentration of Alimet® studied were measured into a sterile tube (three replicates per sample). Sterile water (1 mL) was added to each tube to assure adequate moisture for salmonella multiplication. A final moisture level of 20% was achieved after inoculation. Each test sample was inoculated with 1.0 mL of a diluted TSB broth culture of nalidixic acid (NA) resistant Salmonella (approximately 10^{3.5} cells/g as determined from spread plate counts of the culture). The inoculated samples were mixed with a sterile tongue blade or equivalent tool and incubated at 37° C. for the duration of the trial.

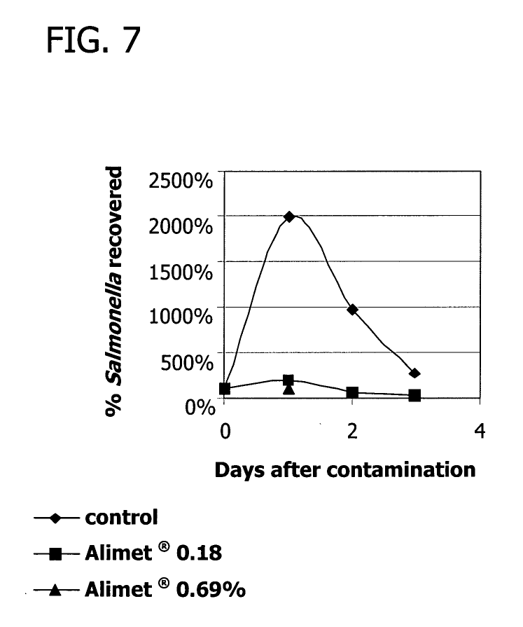

Salmonella counts were determined using brilliant green agar containing sodium nalidixate at days 1, 2 and 3. A 1 g test sample was taken from each tube and transferred to 9 mL sterile water. This test sample was incubated at 4° C. for approximately 4 hours, then agitated for 60-90 seconds. Each test sample was serially diluted in 1:10, 1:100 and 1:1000 proportions, and 100 μL of undiluted test sample, 1:10 diluted sample, 1:100 diluted sample, and 1:1000 diluted samples were plated on brilliant green agar plates containing sodium nalidixate. The percent recovery of Salmonella for different levels of Alimet® is reported in Table 8 and FIG. 7.

TABLE 8
Recovery of Salmonella in meat meal premix
with 20% moisture; 1:10 dilution

| Alimet ® | Alimet ® | | | | |
|---|---|---|---|---|---|
| conc. | conc. | % Salmonella recovered | | | |
| (reported) | (found) | day 0 | day 1 | day 2 | day 3 |
| control (0%) | | 100% | 1990% | 971% | 267% |
| 0.275% | 0.056% | 100% | 102% | 67% | <3%* |
| 0.18% | 0.140% | 100% | 190% | 65% | 11% |
| 0.25% | 0.188% | 100% | 476% | 139% | 25% |
| 0.36% | 0.192% | 100% | 114% | 62% | 4% |
| 0.40% | 0.220% | 100% | 343% | 267% | 53% |
| 0.69% | 0.631% | 100% | 5% | <3%* | <3%* |

*below detection limit

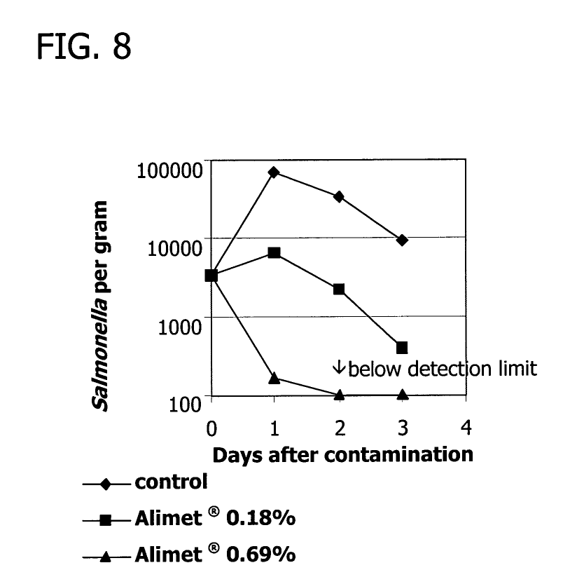

The control sample (no Alimet® added) showed an initial steep increase in Salmonella population one day after inoculation, indicating multiplication of the bacteria. This multiplication was followed by a gradual decline in the bacterial counts on days 2 and 3. Results from the highest level of Alimet® tested suggest that Alimet® is bactericidal for Salmonella in meat and bone meal. As FIG. 8 illustrates, Alimet® at this level gives results comparable to treatment with formic acid at 1.65% (15 kg/ton). See Liu, “Using Organic Acids to Control Salmonella in Poultry Production,” Kemin Industries (Asia) Pte Limited, Singapore, available at http://www.kemin.com.

Alimet® in the range of 0.14-0.22% likewise showed anti-Salmonella effects, and does not appear to be dose-dependent in this range. However, each sample was from different batches of MBM, which may be responsible for the lack of dose-response. The initial multiplication seen in the control was significantly reduced upon addition of Alimet® at these levels. The subsequent decline occurred faster for these lower levels of Alimet® compared to the control.

### EXAMPLE 7

Fungal growth in basal starter mash was studied in compositions supplemented with DLM or Alimet® as the methionine source. Total microbial growth was monitored by measuring carbon dioxide (CO₂) formation in sealed vessels at 28° C. over time. Measurement of CO₂ formation does not distinguish between bacterial and mold growth; however, the ability of mold to grow at much lower water activities, compared to bacteria, is well known, and both mold and bacteria play a part in feed degradation.

The technique of using a closed system and measuring CO₂ formation has been verified as an approximation of the conditions found in grain storage bins. See, e.g., Muir et al., Trans. ASAE, 28(5) 1673-1675 (1985), the contents of which are hereby incorporated by reference in their entirety.

A mash starter mash feed (formulation shown in Table 9, below) was subdivided into three groups: basal (control), 0.2% DLM, or 0.2% Alimet®. The feed studied had no commercial mold inhibitors added, and is representative of a typical broiler feed.

Initial moisture of the feed was 10.8%. After the addition of Alimet® or DLM, the moisture of the samples was adjusted by the addition of 2%, 4%, or 6% sterile distilled water to promote mold growth, achieving three moisture groups: 83.2% dry matter/12.8% moisture; 85.2% dry matter/14.8% moisture; and 87.2% dry matter/16.8% moisture.

For each study, four replicate samples of each moisture group were mixed, and 600 g of the mixture was placed into lL containers, sealed, and placed at 28° C. in a temperature-controlled room. Draeger Detector tubes (CO₂-measuring, obtained from Fisher Scientific) were used to measure the developed CO₂ at different days following vessel sealing (two measurements were made per week). Draeger Short-Term Detector Tubes are glass tubes filled with inert carrier and an indicating reagent. The reagent produces a colorimetric indication in the presence of a particular gas (CO₂). The concentration of gas is read directly from the discoloration on the tube's printed scale.

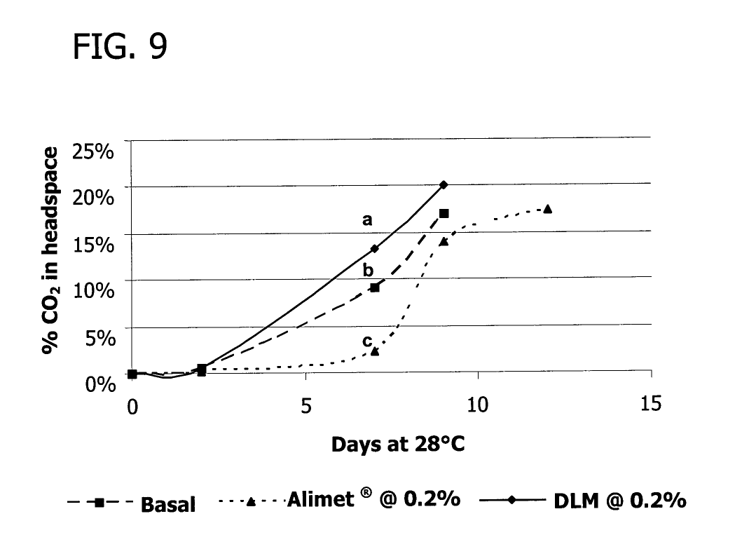

The specific mold species present were not identified. Statistical analysis was accomplished by using Duncan's multiple range test (SAS). Different letters on individual time points in FIGS. 9-11 indicate statistical differences of P<0.05.

TABLE 9
Basal starter mash formulation

| | % by weight |
|---|---|
| Ingredient | of total mix |
| Corn | 60.551 |
| Soybean meal | 32.254 |
| Fat, animal | 3.665 |
| Dical940224PhosfromD | 1.861 |
| (dicalcium phosphate) | |
| Limestone | 0.811 |
| Novus Vitamin/Mineral premix manufactured | 0.350 |
| by Trouw Nutrition (Highland, Illinois) | |
| Salt | 0.340 |
| L-lysine HCl 78% | 0.097 |
| Threonine | 0.051 |
| Santoquin-mix6 | 0.019 |
| Antioxidant preservative sold by Solutia | |
| Inc. (St. Louis, Missouri) | |
| Copper Sulfate | 0.003 |

As shown in FIG. 9, Alimet® effectively inhibited mold growth for up to seven days at the highest moisture level tested (83.2% dry matter/16.8% moisture), while DLM was the least effective, and, in fact, showed mold growth within two days. In fact, DLM-treated starter mash showed mold growth faster than basal mash (i.e., feed with no added methionine or methionine analog), and faster than the Alimet®-treated mash, for all moisture levels tested.

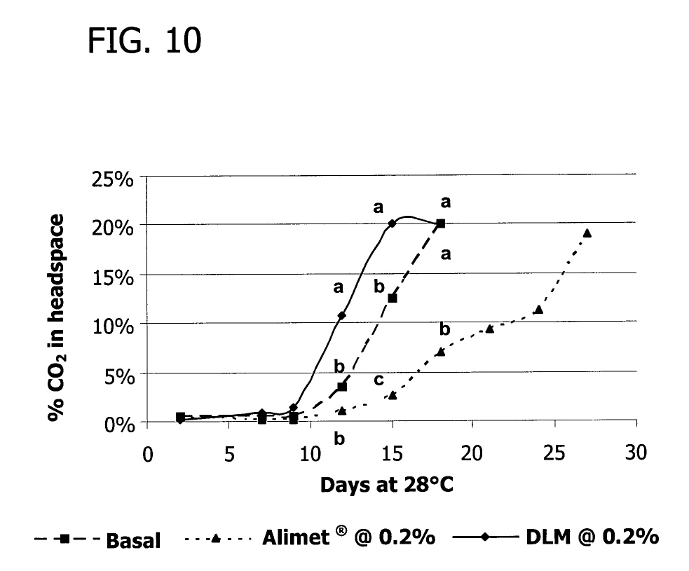

As demonstrated in FIG. 10, Alimet®-treated feed showed a slower rate of mold growth in 85.2% dry matter/14.8% moisture feed, than the DLM-treated feed.

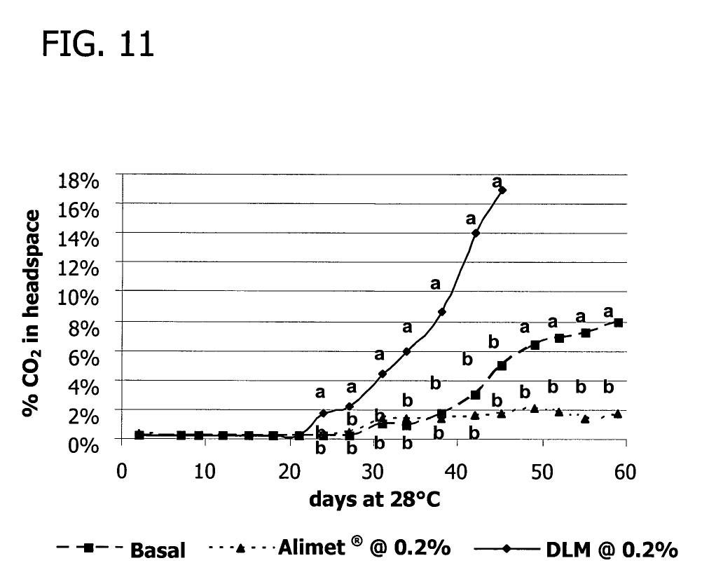

For feed having 87.2% dry matter/12.8% moisture, Alimet®-treated feed shows low mold levels for up to sixty days, while DLM-treated feed shows a sharp increase in mold growth after only twenty days (see FIG. 11).

FIGS. 9, 10, and 11 each illustrate that DLM treated mash is more likely to develop mold than either methionine-deficient feed or feed treated with Alimet®, and that Alimet® was more effective in inhibiting mold growth than compared to untreated feed, or feed supplemented with DLM.

### EXAMPLE 8

The experiment described above in Example 7 was repeated with blends of 2.0 lb/ton, 1.5 lb/ton, 1.0 lb/ton or 0.5 lb/ton of 65% propionic acid and either 2% Alimet® or 2% DLM. The blends were prepared according to the matrix outlined in Table 8, below. The 65% propionic acid was buffered with ammonium hydroxide to a pH of 5.5.

TABLE 10
Antifungal blends of Alimet ® or DLM with propionic acid

| | Alimet ® | Propionic | DLM |
|---|---|---|---|
| Trial No. | (%) | (lb/ton) | (%) |
| 1 | 0.2 | — | — |
| 2 | — | 2 | 0.2 |
| 3 | — | 1.5 | 0.2 |
| 4 | — | 1.0 | 0.2 |
| 5 | — | 0.5 | 0.2 |
| 6 | — | — | 0.2 |
| 7 | 0.2 | 2 | — |
| 8 | 0.2 | 1.5 | — |
| 9 | 0.2 | 1.0 | — |
| 10 | 0.2 | 0.5 | — |

Statistical analysis was accomplished by using Duncan's multiple range test (SAS). Different letters on individual time points in graphs indicate statistical differences of P<0.05.

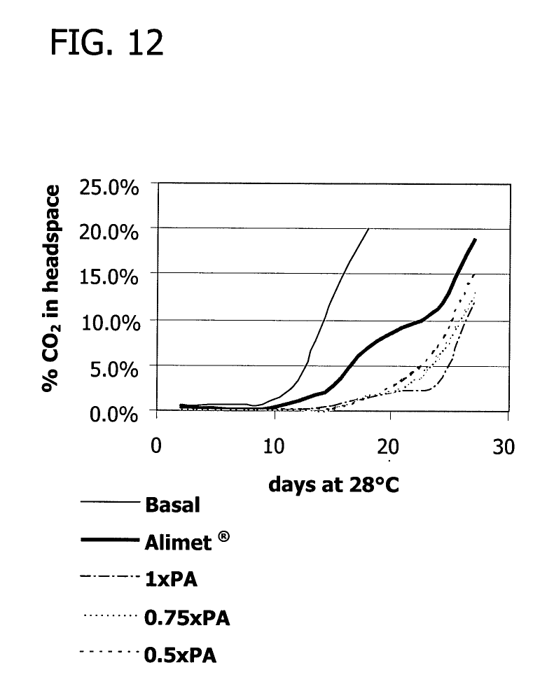

As shown in FIG. 12, basal diet having 85.2% dry matter and containing 0.2% Alimet® delayed the onset of mold growth by 5 days; the effect of 2.0 lb/ton, 1.5 lb/ton and 1.0 lb/ton 65% propionic acid is about 11 days, i.e, no significant difference was seen when propionic acid was added beyond 1 lb/ton.

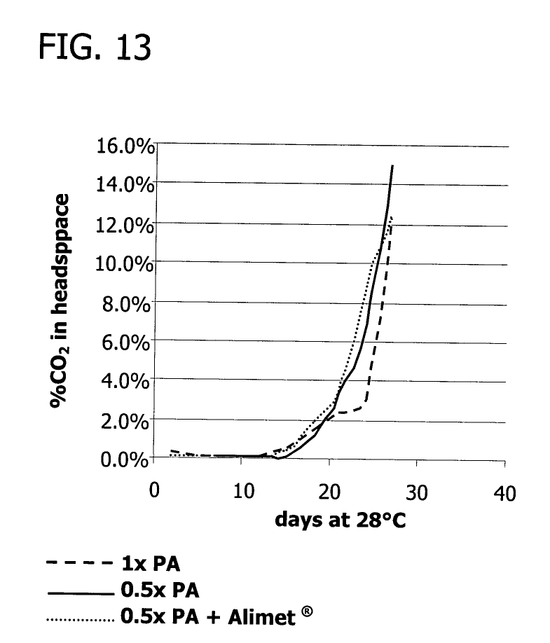

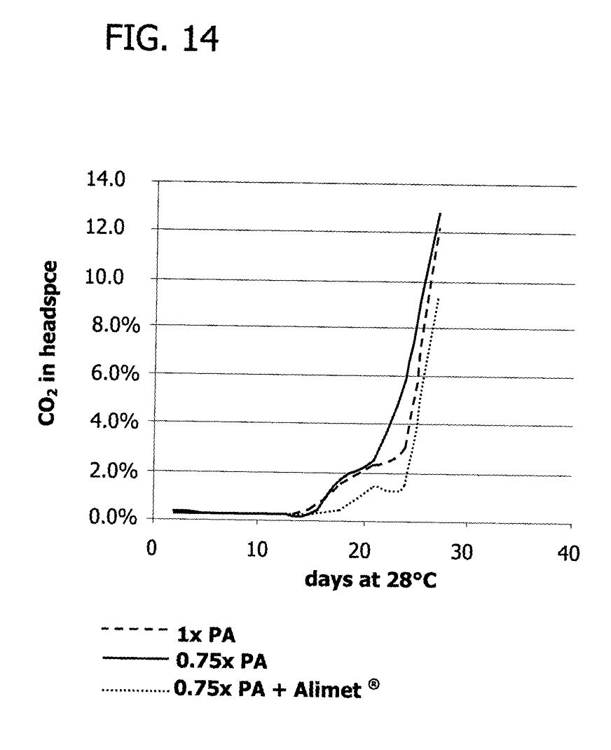

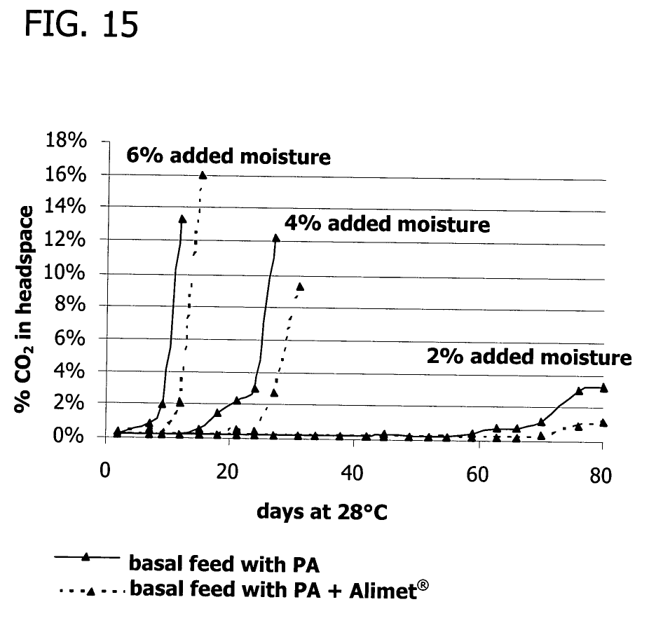

Combinations of Alimet® and propionic acid were compared to feed treated with propionic acid alone, and the results are shown in FIGS. 13-15. The results indicate that, for all moisture levels studied, basal feed containing Alimet® plus propionic acid showed improved mold inhibition compared to feed containing only propionic acid.

### EXAMPLE 9

The effect of formic, butyric and lactic acids on Salmonella populations in basal corn soy based broiler starter feed was studied to determine the levels of these acids required for complete bacteriacide.

Tests were carried out using 1 g feed (crumble ground) with 6% meat and bone meal (“MBM”). Aqueous 25% solutions of formic, butyric, and lactic acids were prepared. The acid solutions were added to the feed as indicated below; water (1 mL) and 150 mM HCl (1.8 mL) was added to bring the pH to 4.0. Naldixic Acid resistant Salmonella (provided by Dr. Stan Bailey, USDA/ARS, Athens, Ga.) (initial colony count=40,000 cfu/g) was added, and the feed solutions were incubated at 37° C. for 90 min. Each sample was diluted with 6 mL H₂O, plated, and counted the following day. Colony counts are reported in Table 11 below.

TABLE 11
Effect of formic, butyric or lactic acids on
Salmonella populations in feed
[Δ log = logₛₐₘₚₗₑ − log_{control}]

| Acid | g/L | vol. (μL) | final pH | log cfu/g | Δ log |
|---|---|---|---|---|---|
| control | | | 4.47 | 5.0 | — |
| formic | 2.5 | 10 | 4.28 | 4.3 | 0.7 |
| | 5.0 | 20 | 4.18 | 3.6 | 1.4 |
| | 7.5 | 30 | 4.1 | 1.0 | 4.0 |
| | 10 | 40 | 4 | 1.0 | 4.0 |
| butyric | 10 | 40 | 4.31 | 4.3 | 0.7 |
| | 30 | 120 | 4.17 | 1.0 | 4.0 |
| | 50 | 200 | 4.04 | 1.0 | 4.0 |
| lactic | 10 | 40 | 4.2 | 4.1 | 0.9 |
| | 30 | 120 | 3.92 | 1.0 | 4.0 |
| | 50 | 200 | 3.68 | 1.0 | 4.0 |

| | |
|---|---|
| control | 4.44 |

Complete bacteriacide was seen at the two highest doses tested for all three acids (7.5 and 10 g/L for formic acid; 30 and 50 g/L for butyric and lactic acids).

### EXAMPLE 10

Following the procedure set forth in Example 9, the effect of blends of formic, butyric and/or lactic acids, with and without Alimet®, on Salmonella counts in basal corn soy based broiler starter feed (described in Table 9, above, crumble feed with 6% MBM) was studied. The blends studied are described in Table 12, and the results obtained in the in vitro study are reported in Tables 13-15.

TABLE 12
Acid blend formulations (concentrations reported in g/L)

| | Formic | Lactic | Butyric | |
|---|---|---|---|---|
| Blend | Acid | Acid | Acid | Alimet ® |
| A1 | 5 | — | — | — |
| A2 | 4 | 4 | — | — |
| A3 | 4 | — | 4 | — |
| A4 | 3 | — | 8 | — |
| A5 | 3 | 4 | 4 | — |
| A6 | 3 | 8 | — | — |
| A7 | 2 | — | 12 | — |
| A8 | 2 | 4 | 8 | — |
| A9 | 2 | 8 | 4 | — |
| A10 | 2 | 12 | — | — |
| A11 | 1 | — | 16 | — |
| A12 | 1 | 4 | 12 | — |
| A13 | 1 | 8 | 8 | — |
| A14 | 1 | 12 | 4 | — |
| A15 | 1 | 16 | — | — |
| A16 | — | — | 20 | — |
| A17 | — | 4 | 16 | — |
| A18 | — | 8 | 12 | — |
| A19 | — | 12 | 8 | — |
| A20 | — | 16 | 4 | — |
| A21 | — | 20 | — | — |
| A22 | 5 | — | — | 1 |
| A23 | 4 | 4 | — | 1 |
| A24 | 4 | — | 4 | 1 |
| A25 | 3 | — | 8 | 1 |
| A26 | 3 | 4 | 4 | 1 |
| A27 | 3 | 8 | — | 1 |
| A28 | 2 | — | 12 | 1 |
| A29 | 2 | 4 | 8 | 1 |
| A30 | 2 | 8 | 4 | 1 |
| A31 | 2 | 12 | — | 1 |
| A32 | 1 | — | 16 | 1 |
| A33 | 1 | 4 | 12 | 1 |
| A34 | 1 | 8 | 8 | 1 |
| A35 | 1 | 12 | 4 | 1 |
| A36 | 1 | 16 | — | 1 |
| A37 | — | — | 20 | 1 |
| A38 | — | 4 | 16 | 1 |
| A39 | — | 8 | 12 | 1 |
| A40 | — | 12 | 8 | 1 |
| A41 | — | 16 | 4 | 1 |
| A42 | — | 20 | — | 1 |
| A43 | 5 | — | — | 2.27 |
| A44 | 4 | 4 | — | 2.27 |
| A45 | 4 | — | 4 | 2.27 |
| A46 | 3 | — | 8 | 2.27 |
| A47 | 3 | 4 | 4 | 2.27 |
| A48 | 3 | 8 | — | 2.27 |
| A49 | 2 | — | 12 | 2.27 |
| A50 | 2 | 4 | 8 | 2.27 |
| A51 | 2 | 8 | 4 | 2.27 |
| A52 | 2 | 12 | — | 2.27 |
| A53 | 1 | — | 16 | 2.27 |
| A54 | 1 | 4 | 12 | 2.27 |
| A55 | 1 | 8 | 8 | 2.27 |
| A56 | 1 | 12 | 4 | 2.27 |
| A57 | 1 | 16 | — | 2.27 |
| A58 | — | — | 20 | 2.27 |
| A59 | — | 4 | 16 | 2.27 |
| A60 | — | 8 | 12 | 2.27 |
| A61 | — | 12 | 8 | 2.27 |
| A62 | — | 16 | 4 | 2.27 |
| A63 | — | 20 | — | 2.27 |

TABLE 13
Effect of formic/butyric/lactic blends without Alimet ® on
Salmonella populations in feed

| | | | Δ log |
|---|---|---|---|
| Blend | Final pH | log cfu/g | reduction |
| A1 | 4.12 | 3.1 | 1.6 |
| A2 | 4.13 | 2.9 | 1.8 |
| A3 | 4.18 | 3.1 | 1.6 |
| A4 | 4.18 | 3.0 | 1.7 |
| A5 | 4.18 | 3.2 | 1.5 |
| A6 | 4.15 | 2.6 | 2.1 |
| A7 | 4.15 | 2.8 | 1.9 |
| A8 | 4.18 | 2.8 | 1.9 |
| A9 | 4.16 | 2.5 | 2.2 |
| A10 | 4.12 | 1.8 | 2.9 |
| A11 | 4.16 | 2.7 | 2.0 |
| A12 | 4.17 | 2.7 | 2.0 |
| A13 | 4.16 | 2.8 | 1.9 |
| A14 | 4.14 | 2.9 | 1.8 |
| A15 | 4.1 | 2.6 | 2.1 |
| A16 | 4.16 | 2.7 | 2.0 |
| A17 | 4.21 | 2.7 | 2.0 |
| A18 | 4.18 | 2.5 | 2.2 |
| A19 | 4.16 | 1.7 | 3.0 |
| A20 | 4.13 | 2.7 | 2.0 |
| A21 | 4.05 | 1.8 | 2.9 |
| control | 4.33 | 4.7 | — |

TABLE 14
Effect of formic/butyric/lactic blends with 1 g/L added
Alimet ® on Salmonella populations in feed

| | | | Δ log |
|---|---|---|---|
| Blend | Final pH | log cfu/g | reduction |
| A22 | 4.09 | 3.5 | 1.0 |
| A23 | 4.09 | 2.1 | 2.4 |
| A24 | 4.09 | 2.9 | 1.6 |
| A25 | 4.12 | 2.2 | 2.3 |
| A26 | 4.11 | 2.3 | 2.2 |
| A27 | 4.1 | 1.9 | 2.6 |
| A28 | 4.11 | 2.3 | 2.2 |
| A29 | 4.14 | 2.3 | 2.2 |
| A30 | 4.1 | 1.9 | 2.6 |
| A31 | 4.06 | 1.6 | 2.9 |
| A32 | 4.15 | 2.4 | 2.1 |
| A33 | 4.15 | 2.3 | 2.2 |
| A34 | 4.15 | 1.4 | 3.1 |
| A35 | 4.11 | 1.7 | 2.8 |
| A36 | 4.06 | 1.8 | 2.7 |
| A37 | 4.13 | 2.0 | 2.5 |
| A38 | 4.16 | 2.0 | 2.5 |
| A39 | 4.13 | 1.8 | 2.7 |
| A40 | 4.12 | 1.8 | 2.7 |
| A41 | 4.08 | 1.7 | 2.8 |
| A42 | 4.06 | 1.7 | 2.8 |
| control | 4.33 | 4.7 | 0.2 |
| control | 4.36 | 4.5 | — |

TABLE 15
Effect of formic/butyric/lactic blends with 2.27 g/L added
Alimet ® on Salmonella populations in feed

| | | | Δ log |
|---|---|---|---|
| Blend | Final pH | log cfu/g | reduction |
| A43 | 4.21 | 2.8 | 0.7 |
| A44 | 4.17 | 2.8 | 0.7 |
| A45 | 4.18 | 3.0 | 0.5 |
| A46 | 4.18 | 2.8 | 0.7 |
| A47 | 4.15 | 3.7 | −0.2 |
| A48 | 4.11 | 2.0 | 1.5 |
| A49 | 4.19 | 2.7 | 0.8 |
| A50 | 4.19 | 2.8 | 0.7 |
| A51 | 4.16 | 2.8 | 0.7 |
| A52 | 4.13 | 2.4 | 1.1 |
| A53 | 4.2 | 2.8 | 0.7 |
| A54 | 4.2 | 2.7 | 0.8 |
| A55 | 4.14 | 2.3 | 1.2 |
| A56 | 4.13 | 1.7 | 1.8 |
| A57 | 4.08 | 1.0 | 2.5 |
| A58 | 4.21 | 2.3 | 1.2 |
| A59 | 4.23 | 2.7 | 0.8 |
| A60 | 4.17 | 2.0 | 1.5 |
| A61 | 4.14 | 2.0 | 1.5 |
| A62 | 4.12 | 0.7 | 2.8 |
| A63 | 4.07 | 0.7 | 2.8 |
| control | 4.37 | 3.5 | 0 |
| control | 4.45 | 3.5 | — |

Addition of 1 g/L Alimet® to the blend gave improved results in sixteen of the blends tested.

The formulations of the blends used in Examples 11-13 are set forth in Table 16.

TABLE 16
Organic acid/Alimet ® blend formulations

| Blend | Alimet ® | Phos.¹ | Butyric | Formic | Lactic | Prop.² |
|---|---|---|---|---|---|---|
| A64 | 30 | 30 | 20 | — | 20 | — |
| A65 | 30 | — | — | 50 | — | 20 |
| A66 | 20 | 20 | 12 | 30 | 18 | — |
| A67 | 10 | 10 | — | 75 | — | 5 |
| A68 | 30 | 35 | 15 | — | 20 | — |
| A69 | 30 | 5 | — | 55 | — | 10 |
| A70 | 20 | 20 | 12 | — | 18 | 30 |
| A71 | 10 | 5 | 5 | 75 | — | 5 |
| A72 | — | — | — | 50 | — | 50 |
| A73 | — | — | — | 75 | — | 25 |

¹Phosphoric acid
²Propionic acid

### EXAMPLE 11

The effects of blends of organic acids on the colony counts of Salmonella in a corn soy based diet as set forth in Table 9, above, (DLM at 0.2%) were studied, following the procedure outlined in Example 9. The results are reported in Table 17.

TABLE 17
effect of acid blends on Salmonella
populations in corn soy based diet

| | | after 60 min. | | |
|---|---|---|---|---|
| | | | | Δ log |
| Blend | g/kg | cfu/g^{1,2} | log cfu/g | reduction |
| control | | 40,000 | 4.6 | — |
| A64 | 5 | 29,700 | 4.5 | 0.1 |
| | 10 | 2,300 | 3.4 | 1.2 |
| A65 | 5 | 3,100 | 3.5 | 1.1 |
| | 10 | 40 | 1.6 | 3.0 |
| A66 | 5 | 10,000 | 4.0 | 0.6 |
| | 10 | 40 | 1.6 | 3.0 |
| A67 | 5 | 3,000 | 3.5 | 1.1 |
| | 10 | 40 | 1.6 | 3.0 |

| | | | |
|---|---|---|---|
| control | 13,900 | 4.1 | — |

| | | after 90 min. | | | |
|---|---|---|---|---|---|
| | | | | Δ log | |
| Blend | g/kg | cfu/g^{1,2} | log cfu/g | reduction | final pH |
| control | | 46,000 | 4.7 | — | 4.68 |
| A64 | 5 | 19,300 | 4.3 | 0.4 | 4.45 |
| | 10 | 2,400 | 3.4 | 1.3 | 4.32 |
| A65 | 5 | 200 | 2.3 | 2.4 | 4.44 |
| | 10 | 40 | 1.6 | 3.1 | 4.25 |
| A66 | 5 | 13,500 | 4.1 | 0.6 | 4.37 |
| | 10 | 40 | 1.6 | 3.1 | 4.22 |
| A67 | 5 | 200 | 2.3 | 2.4 | 4.33 |
| | 10 | 40 | 1.6 | 3.1 | 4.02 |

| | | | | |
|---|---|---|---|---|
| control | 38,800 | 4.6 | — | 4.68 |

¹40 cfu/g (minimum detection level) reported when no Salmonella detected.
²Single reading for each treatment.

Three of the four blends tested showed complete bacteriacide at 10 g/kg (1%) application. Both the A65 and A67 blends showed significant bacteriacide at the lower application rate (0.5%)

### EXAMPLE 12

The effect of blends of organic acids on Salmonella counts were studied using model poultry and swine diets. Blends A70 and A71 were tested using the corn soy based diet set forth in Table 9, above, with DLM added at 0.2%. The model poultry diet was a corn soy based layer diet, no meat product; the effects of blends A68 and A69 were tested with this diet.

Blends were added to 1 g of feed sample. Salmonella (200 μL, 40,000 cfu) were added to each feed sample, and mixed. The samples were incubated at room temperature, then diluted 1:10 with water and plated on BG plate. Results are reported in Table 18.

TABLE 18
effect of acid blends on Salmonella
populations in corn soy based diet

| | | Final | after 1 h | | after 24 h | |
|---|---|---|---|---|---|---|
| blend | g/kg | pH | cfu/g | log cfu/g | cfu/g | log cfu/g |
| control | | 5.83 | 12,400 | 4.1 | 10,133 | 4.0 |
| (Diet #1) | | | | | | |
| A70 | 2 | 5.53 | 15,000 | 4.2 | 3,300 | 3.5 |
| | 5 | 5.32 | 3,900 | 3.6 | 1,200 | 3.1 |
| | 7.5 | 5.08 | 3,400 | 3.5 | 700 | 2.8 |
| | 10 | 4.94 | 1,500 | 3.2 | 100 | 2.0 |
| A71 | 2 | 5.42 | 9,600 | 4.0 | 2,300 | 3.4 |
| | 5 | 5.16 | 1,300 | 3.1 | 100 | 2.0 |
| | 7.5 | 4.84 | 200 | 2.3 | 100 | 2.0 |
| | 10 | 4.66 | 100 | 2.0 | 100 | 2.0 |
| control | | 5.92 | 15,300 | 4.2 | 6,300 | 3.8 |
| (Diet #2) | | | | | | |
| A68 | 2 | 5.70 | 10,400 | 4.0 | 5,600 | 3.7 |
| | 5 | 5.67 | 6,300 | 3.8 | 4,200 | 3.6 |
| | 7.5 | 5.54 | 9,900 | 4.0 | 2,600 | 3.4 |
| | 10 | 5.36 | 8,300 | 3.9 | 1,300 | 3.1 |
| A69 | 2 | 5.53 | 7,800 | 3.9 | 3,300 | 3.5 |
| | 5 | 5.29 | 3,600 | 3.6 | 1,200 | 3.1 |
| | 7.5 | 5.22 | 1,200 | 3.1 | 300 | 2.5 |
| | 10 | 5.08 | 1,000 | 3.0 | 100 | 2.0 |

The antibacterial effect of two organic acid/Alimet® blends were compared with blends containing formic and propionic acids, and with no Alimet®, following the procedure set forth in Example 12. Results after 90 minutes are reported in Table 19.

TABLE 19

| Blend | g/kg | cfu/g | log cfu/g | final pH |
|---|---|---|---|---|
| control | | 29,400 | 4.5 | 4.54 |
| A65 | 2 | 140 | 2.1 | 4.56 |
| | 5 | 80 | 1.9 | 4.45 |
| | 10 | 1 | 0 | 4.32 |
| A67 | 2 | 900 | 3.0 | 4.57 |
| | 5 | 1,100 | 3.0 | 4.49 |
| | 10 | 1 | 0 | 4.25 |
| A72 | 2 | 2,100 | 3.3 | 4.5 |
| | 5 | 90 | 2 | 4.39 |
| | 10 | 1 | 0 | 4.15 |
| A73 | 2 | 2,700 | 3.4 | 4.51 |
| | 5 | 600 | 2.8 | 4.4 |
| | 10 | 1 | 0 | 4.1 |

| | | | |
|---|---|---|---|
| control | 30,650 | 4.5 | 4.68 |

### EXAMPLE 14

The antibacterial effect of two different batches of Alimet® on a corn soy diet (see Table 9, above) were compared. The first batch was of an unknown age and the second batch was freshly prepared (less than two weeks old). The protocol set forth in Example 9 was used, and results are reported in Table 20.

TABLE 20
effect of two different batches of Alimet ® on Salmonella
populations in corn soy based diet after 90 min. incubation

| | g/kg | | | |
|---|---|---|---|---|
| Acid | added | cfu/g | log cfu/g | pH |
| control | | 6,850 | 3.8 | 4.80 |
| Alimet ® | 2.3 | 800 | 2.9 | 4.72 |
| batch 1 | 5.7 | 50 | 1.7 | 4.66 |
| | 8.5 | 120 | 2.1 | 4.58 |
| | 11.4 | 0 | — | 4.57 |
| Alimet ® | 2.3 | 3,900 | 3.6 | 4.68 |
| batch 2 | 5.7 | 300 | 2.5 | 4.54 |
| | 8.5 | 20 | 1.3 | 4.61 |
| | 11.4 | 20 | 1.3 | 4.57 |

| | | | |
|---|---|---|---|
| control | 14,850 | 4.2 | 4.80 |

The first batch of Alimet® showed slightly improved bactericidal effects at lower concentrations. Both batches were bactericidal at higher doses.

### EXAMPLE 15

The antibacterial effect of dry acids (fumaric, tartaric, and sorbic) alone and in combination with Alimet® blends were studied according to the protocol of Example 9. The formulations of the blends studied are reported in Table 21. The results after 90 minutes are reported in Table 22.

TABLE 21

acid concentration (g/kg)

| Blend | Alimet ® | Fumaric | Tartaric | Sorbic |
|---|---|---|---|---|
| A74 | 10 | 0 | 0 | 0 |
| A75 | 0 | 10 | 0 | 0 |
| A76 | 0 | 0 | 10 | 0 |
| A77 | 0 | 0 | 0 | 10 |
| A78 | 5 | 5 | 0 | 0 |
| A79 | 5 | 0 | 5 | 0 |
| A80 | 5 | 0 | 0 | 5 |

TABLE 22

| | | | Δ log |
|---|---|---|---|
| Blend | cfu/g | log cfu/g | reduction |
| control | 27900 | 4.4 | |
| A74 | 40 | 1.6 | 2.8 |
| A75 | 30 | 1.5 | 3.0 |
| A76 | 14450 | 4.2 | 0.3 |
| A77 | 1150 | 3.1 | 1.4 |
| A78 | 0 | — | 4.4 |
| A79 | 4600 | 3.7 | 0.8 |
| A80 | 170 | 2.2 | 2.2 |
| control | 13900 | 4.1 | |

### EXAMPLE 16

The effect of formic acid on Lactobacillus plantarum was studied. As demonstrated in Example 1, addition of Alimet® at pH 3.5 to a bacteria-containing broth showed a clear lethal effect on L. plantarum at doses of 3 and 5 g/l. Comparable concentrations of formic acid (technical quality 85%, ex Franklin Products) were also studied and compared against Alimet®.

Fresh overnight culture L. plantarum in Brain Heart Infusion broth is used to inoculate medium 5 at a log 4.1 cfu/mL at pH 3.5. The tubes are incubated under oxygen reduced atmosphere for 6 hours at 37° C. Colony counts are performed according standard procedures. All analyses were performed in duplicate, and the results are reported in Table 23.

TABLE 23
Effect of Alimet ® and formic acid on L. plantarum colony
counts in broth (after 6 hours)
initial log cfu/mL = 5.61
Δ log = log_{6 hours} − logᵢₙᵢₜᵢₐₗ

| Acid | g/kg | log cfu/mL | Δ log |
|---|---|---|---|
| control | | 5.67 | 0.06 |
| Alimet ® | 3 | 3.02 | −2.59 |
| | 5 | 0.83 | −4.78 |
| formic acid | 3 | 3.19 | −2.42 |
| | 5 | −0.30 | −5.91 |

### EXAMPLE 17

Blends of various acids with Alimet® were studied at pH 4.5. The effect of these blends on S. enteritidis colony counts in broth were studied, using the protocol set out in Example 1. Results are reported in Tables 24-26.

TABLE 24
Effects of blends of acids and Alimet ® on
S. enteritidis colony counts in broth

| Blend | Acid | Acid g/L | Alimet ® g/L | log cfu/mL |
|---|---|---|---|---|
| Control | | | | 5.04 |
| Control (Alimet ®) | | | | −0.30 |
| A81 | formic | 5 | 0 | 3.93 |
| A82 | | 4.5 | 0.5 | 0.74 |
| A83 | | 4 | 1 | 0.24 |
| A84 | | 3.75 | 1.25 | 0.15 |
| A85 | | 3.5 | 1.5 | −0.15 |
| A86 | | 2.5 | 2.5 | −0.30 |
| A87 | butyric | 5 | 0 | 4.44 |
| A88 | | 4.5 | 0.5 | 3.56 |
| A89 | | 4 | 1 | 1.70 |
| A90 | | 3.75 | 1.25 | 1.44 |
| A91 | | 3.5 | 1.5 | −0.15 |
| A92 | | 2.5 | 2.5 | −0.30 |
| A93 | citric | 5 | 0 | 4.74 |
| A94 | | 4.5 | 0.5 | 4.40 |
| A95 | | 4 | 1 | 4.34 |
| A96 | | 3.75 | 1.25 | 1.59 |
| A97 | | 3.5 | 1.5 | 1.19 |
| A98 | | 2.5 | 2.5 | 0.24 |

TABLE 25
Effects of blends of acids or formaldehyde and Alimet ® on
S. enteritidis colony counts in broth

| Blend | Acid | Acid g/L | Alimet ® g/L | log cfu/mL |
|---|---|---|---|---|
| Control | | | | 5.02 |
| Control (Alimet ®) | | | | 0.00 |
| A99 | fumaric | 5 | 0 | 4.68 |
| A100 | | 4.5 | 0.5 | 4.65 |
| A101 | | 4 | 1 | 4.43 |
| A102 | | 3.75 | 1.25 | 4.38 |
| A103 | | 3.5 | 1.5 | 4.36 |
| A104 | | 2.5 | 2.5 | 2.32 |
| A105 | lactic | 5 | 0 | 4.44 |
| A106 | | 4.5 | 0.5 | 4.48 |
| A107 | | 4 | 1 | 4.43 |
| A108 | | 3.75 | 1.25 | 4.51 |
| A109 | | 3.5 | 1.5 | 4.75 |
| A110 | | 2.5 | 2.5 | 4.55 |
| A111 | malic | 5 | 0 | 5.01 |
| A112 | | 4.5 | 0.5 | 4.71 |
| A113 | | 4 | 1 | 4.77 |
| A114 | | 3.75 | 1.25 | 4.91 |
| A115 | | 3.5 | 1.5 | 4.96 |
| A116 | | 2.5 | 2.5 | 4.89 |

TABLE 26
Effects of blends of acids or formaldehyde and Alimet ® on
S. enteritidis colony counts in broth

| Blend | Acid | Acid g/L Alimet ® g/L | | log cfu/mL |
|---|---|---|---|---|
| Control | | | | 4.95 |
| Control (Alimet ®) | | | | 0.63 |
| A117 | propionic | 5 | 0 | 4.34 |
| A118 | | 4.5 | 0.5 | 4.34 |
| A119 | | 4 | 1 | 4.10 |
| A120 | | 3.75 | 1.25 | 3.76 |
| A121 | | 3.5 | 1.5 | 3.30 |
| A122 | | 2.5 | 2.5 | 0.87 |
| A123 | phosphoric | 5 | 0 | 4.68 |
| A124 | | 4.5 | 0.5 | 4.67 |
| A125 | | 4 | 1 | 4.56 |
| A126 | | 3.75 | 1.25 | 4.62 |
| A127 | | 3.5 | 1.5 | 4.48 |
| A128 | | 2.5 | 2.5 | 3.30 |
| A129 | formaldehyde | 5 | 0 | −0.30 |
| A130 | | 4.5 | 0.5 | −0.30 |
| A131 | | 4 | 1 | −0.30 |
| A132 | | 3.75 | 1.25 | −0.30 |
| A133 | | 3.5 | 1.5 | −0.30 |
| A134 | | 2.5 | 2.5 | −0.30 |

Phosphoric, fumaric, lactic, malic and propionic acids do not show a significant inhibitory effect at 5 g/l. Blends of these acids with Alimet® gave similar results, except for the 50:50 blend of fumaric and Alimet®, which gave greater than 2 log reduction in colony counts compared to 5 g/L of fumaric alone, and the 50:50 blend of phosphoric and Alimet®, which gave an approximately 1.3 log reduction compared to 5 g/L of phosphoric alone.

Blends of formic acid and Alimet® performed more favorably than formic acid alone for all the blends studied. Similarly, blends of butyric acid and Alimet®, and citric acid with Alimet®, gave improved bactericidal effects with increasing proportion of Alimet® added.

### EXAMPLE 18

The antibacterial effect of acid blends was studied according to the protocol of Example 9. Phosphoric acid (75%) was obtained from Astaris (St. Louis, Mo.), lot #TK60. L-lactic acid (80%) was obtained from Purac America (Lincolnshire, Ill.), batch #015703-A. Butyric acid (99+%) was obtained from Aldrich Chemical Co. (Milwaukee, Wis.), batch #0.5110A. The formulations of the blends studied are reported in Table 27. The results are reported in Table 28.

TABLE 27

acid formulations (% of total)

| Blend | Alimet ® | Lactic | Phosphoric | Butyric |
|---|---|---|---|---|
| A135 | 0.33 | 0.67 | — | — |
| A136 | 0.317 | 0.633 | 0.05 | — |
| A137 | 0.267 | 0.533 | 0.20 | — |
| A138 | 0.25 | 0.50 | 0.25 | — |
| A139 | 0.33 | 0.33 | — | 0.33 |
| A140 | 0.317 | 0.317 | 0.05 | 0.317 |
| A141 | 0.267 | 0.267 | 0.20 | 0.267 |
| A142 | 0.25 | 0.25 | 0.25 | 0.25 |
| A143 | 0.33 | — | — | 0.67 |
| A144 | 0.317 | — | 0.05 | 0.633 |
| A145 | 0.267 | — | 0.20 | 0.533 |
| A146 | 0.25 | — | 0.25 | 0.50 |

TABLE 28

| Blend | cfu/g | log cfu/g | final pH |
|---|---|---|---|
| control | 7,900 | 3.9 | 4.86 |
| A135 | 20 | 1.3 | 4.49 |
| A136 | 20 | 1.3 | 4.39 |
| A137 | 340 | 2.5 | 4.52 |
| A138 | 160 | 2.2 | 4.44 |
| A139 | 80 | 1.9 | 4.50 |
| A140 | 20 | 1.3 | 4.49 |
| A141 | 160 | 2.2 | 4.42 |
| A142 | 160 | 2.2 | 4.43 |
| A143 | 160 | 2.2 | 4.48 |
| A144 | 40 | 1.6 | 4.48 |
| A145 | 240 | 2.4 | 4.53 |
| A146 | 20 | 1.3 | 4.42 |
| control | 22,100 | 4.3 | 4.73 |

### EXAMPLE 19

The effects of blend A71 at neutral pH were tested. The blend was added to 1 g of feed sample. Salmonella (40,000 cfu (65 ul)) was added to each 1 g sample, mixed, and incubated at room temperature. Following incubation, samples were diluted 1:10 with water and plated on a BG plate.

Two diets were studied, as shown in Table 29. Results are reported in Table 30.

TABLE 29

| | |
|---|---|
| ingredients | % of total |

Diet 1 (swine diet)

| | |
|---|---|
| corn | 51.60 |
| SBM, 48 | 30 |
| DairyLac 80 | 8.50 |
| Menhaden fish meal (Select) | 3.98 |
| Choice white grease | 3.00 |
| Dicalcium Phosphate | 1.24 |
| Limestone | 0.34 |
| Lysine | 0.26 |
| DL-Methionine | 0.13 |
| Threonine | 0.16 |
| Vitamins, TMs, Salt and Mecadox | 0.93 |
| Corn Starch | to 100 |

Diet 2 (broiler diet)

| | |
|---|---|
| corn | 60.551 |
| SBM | 32.254 |
| Fat, animal | 3.665 |
| Dicalcium Phosphate | 1.861 |
| Limestone | 0.811 |
| Vitamin/Mineral premix | 0.350 |
| Salt | 0.340 |
| L-lysine HCl 78% | 0.097 |
| Threonine | 0.051 |
| Satoquin-mix6 | 0.019 |
| Copper Sulfate | 0.003 |
| DL-methionine | 0.2 |

TABLE 30
Blend tested: A71

| | 1 hour | | 24 hours | | 48 hours | | final |
|---|---|---|---|---|---|---|---|
| g/kg | cfu/g | log | cfu/g | log | cfu/g | log | pH |

Diet 1

| | | | | | | | |
|---|---|---|---|---|---|---|---|
| control | 27,700 | 4.4 | 6,432 | 3.8 | 2,530 | 3.4 | 5.83 |
| 2 | 4,320 | 3.6 | 80 | 1.9 | 40 | 1.6 | 5.52 |
| 5 | 3,840 | 3.6 | 40 | 1.6 | 0 | — | 5.21 |
| 7.5 | 3,080 | 3.5 | 80 | 1.9 | 0 | — | 5.01 |
| 10 | 1,560 | 3.2 | 0 | — | 0 | — | 4.87 |

Diet 2

| | | | | | | | |
|---|---|---|---|---|---|---|---|
| control | 25,000 | 4.4 | 6,160 | 3.8 | 2,570 | 3.4 | 6.06 |
| 2 | 8,080 | 3.9 | 40 | 1.6 | 40 | 1.6 | 5.67 |
| 5 | 7,000 | 3.8 | 40 | 1.6 | 40 | 1.6 | 5.31 |
| 7.5 | 4,400 | 3.6 | 40 | 1.6 | 0 | — | 5.1 |
| 10 | 900 | 3.0 | 0 | — | 0 | — | 4.82 |

### EXAMPLE 20

Following the protocol of Example 19, the effects of blend A69 at neutral pH were tested. Formic acid (85%) was obtained from BASF Corporation (Mt. Olive, N.J.), product #019723, lot #87656216KO. Two diets were studied, as shown in Table 31. Results are reported in Table 32.

TABLE 31

| | |
|---|---|
| ingredients | % of total |

Diet 3

| | |
|---|---|
| soybean meal | 33.40 |
| corn | 32.85 |
| wheat hard red | 20.00 |
| organic peas meal | 5.00 |
| fat, animal | 4.80 |
| dicalcium phophate | 1.81 |
| limestone | 0.98 |
| salt | 0.43 |
| Vitamin/Mineral premix | 0.35 |
| Threonine | 0.10 |
| Avizyme1502 | 0.10 |
| Santoquin-mix6 | 0.02 |
| Coban 60 | 0.05 |
| copper sulfate | 0.00 |

Diet 4

| | |
|---|---|
| corn | 60.50 |
| soybean meal | 28.46 |
| limestone | 7.76 |
| Dicalcium Phosphate | 1.63 |
| animal fat | 1.00 |
| Vitamin/Mineral premix | 0.35 |
| salt | 0.26 |
| Santoquin-mix6 | 0.02 |
| copper sulfate | 0.00 |
| choline Cl-60% | 0.00 |

TABLE 32
blend tested: A69

| | 1 hour | | 24 hours | | 48 hours | | final |
|---|---|---|---|---|---|---|---|
| g/kg | cfu/g | log | cfu/g | log | cfu/g | log | pH |

Diet 3

| | | | | | | | |
|---|---|---|---|---|---|---|---|
| control | 68,000 | 4.8 | 2,470 | 3.4 | 880 | 2.9 | 6.05 |
| 2 | 5,600 | 3.7 | 120 | 2.1 | 40 | 1.6 | 5.67 |
| 5 | 8,900 | 3.9 | 40 | 1.6 | 40 | 1.6 | 5.46 |
| 7.5 | 4,600 | 3.7 | 40 | 1.6 | 80 | 1.9 | 5.28 |
| 10 | 10,600 | 4.0 | 40 | 1.6 | 0 | — | 5.13 |

Diet 4

| | | | | | | | |
|---|---|---|---|---|---|---|---|
| control | 68,000 | 4.8 | 2,630 | 3.4 | 1,380 | 3.1 | 5.99 |
| 2 | 8,600 | 3.9 | 0 | — | 0 | — | 5.8 |
| 5 | 900 | 3.0 | 40 | 1.6 | 0 | — | 5.55 |
| 7.5 | 2,600 | 3.4 | 200 | 2.3 | 0 | — | 5.37 |
| 10 | 2,700 | 3.4 | 0 | — | 40 | 1.6 | 5.12 |

### EXAMPLE 21

Following the protocol of Example 9, the effects of blends of Alimet®, lactic acid, formic acid, and/or butyric acid were studied. Blend formulations are set forth in Table 33. Up to five replicates were performed, and results are reported in Tables 34 and 35.

TABLE 33
acid formulations (g/kg)

| Blend | A¹ | L² | F³ | B⁴ |
|---|---|---|---|---|
| A147 | 0 | 0 | 0 | 10 |
| A148 | 2 | 0 | 0 | 8 |
| A149 | 7.5 | 0 | 0 | 2.5 |
| A150 | 3.3 | 0 | 0 | 6.7 |
| A151 | 2 | 2.67 | 0 | 5.33 |
| A152 | 7.5 | 0.8 | 0 | 1.67 |
| A153 | 0 | 0 | 3.3 | 6.7 |
| A154 | 2 | 0 | 2.67 | 5.3 |
| A155 | 7.5 | 0 | 0.8 | 1.67 |
| A156 | 0 | 6.7 | 0 | 3.3 |
| A157 | 2 | 5.3 | 0 | 2.67 |
| A158 | 7.5 | 1.67 | 0 | 0.8 |
| A159 | 0 | 3.3 | 3.3 | 3.3 |
| A160 | 2 | 2.67 | 2.67 | 2.67 |
| A161 | 7.5 | 0.8 | 0.8 | 0.8 |
| A162 | 0 | 0 | 6.7 | 3.3 |
| A163 | 2 | 0 | 5.3 | 2.67 |
| A164 | 7.5 | 0 | 1.67 | 0.8 |
| A165 | 0 | 10 | 0 | 0 |
| A166 | 2 | 8 | 0 | 0 |
| A167 | 7.5 | 2.5 | 0 | 0 |
| A168 | 0 | 6.7 | 3.3 | 0 |
| A169 | 2 | 5.3 | 2.67 | 0 |
| A170 | 7.5 | 1.67 | 0.8 | 0 |
| A171 | 0 | 3.3 | 6.7 | 0 |
| A172 | 2 | 2.67 | 5.3 | 0 |
| A173 | 7.5 | 0.8 | 1.67 | 0 |
| A174 | 0 | 0 | 10 | 0 |
| A175 | 2 | 0 | 8 | 0 |
| A176 | 7.5 | 0 | 2.5 | 0 |

¹Alimet ®
²Lactic acid
³Formic acid
⁴Butyric acid

TABLE 34

| Trial: | Average | | | 1 | 2 | 3 | 4 |
|---|---|---|---|---|---|---|---|
| Blend | cfu/g | log | Δ log | cfu/g | cfu/g | cfu/g | cfu/g |
| control | 49,200 | 4.7 | — | 58,200 | 43,400 | 46,000 | N/A |
| A147 | 18,800 | 4.3 | 0.4 | 22,200 | 15,400 | N/A | N/A |
| A148 | 15,700 | 4.2 | 0.5 | 19,800 | 11,600 | N/A | N/A |
| A149 | 70 | 1.8 | 2.8 | 80 | 200 | 0 | 0 |
| A150 | 12,300 | 4.1 | 0.6 | 1,200 | 23,400 | N/A | N/A |
| A151 | 6,000 | 3.8 | 0.9 | 8,200 | 3,800 | N/A | N/A |
| A152 | 60 | 1.8 | 2.9 | 40 | 40 | 120 | 40 |
| A153 | 1,600 | 3.2 | 1.5 | 3,200 | 0 | N/A | N/A |
| A154 | 20 | 1.3 | 3.4 | 40 | 40 | 0 | 0 |
| A155 | 10 | 1.0 | 3.7 | 40 | 0 | 0 | 0 |
| A156 | 11,300 | 4.1 | 0.6 | 6,000 | 16,600 | N/A | N/A |
| A157 | 2,900 | 3.5 | 1.2 | 1, 000 | 4,800 | N/A | N/A |
| A158 | 400 | 2.6 | 2.1 | 800 | 0 | N/A | N/A |
| A159 | 0 | — | 4.7 | 0 | 0 | 0 | 0 |
| A160 | 0 | — | 4.7 | 0 | 0 | 0 | 0 |
| A161 | 20 | 1.3 | 3.4 | 40 | 40 | 0 | 0 |
| control | 63,133 | 4.8 | — | 56,800 | 64,000 | 68,600 | N/A |

N/A = replicate not performed

TABLE 35

| Trial: | Average | | | 1 | 2 | 3 | 4 |
|---|---|---|---|---|---|---|---|
| Blend | cfu/g | log | Δ log | cfu/g | cfu/g | cfu/g | cfu/g |
| control | 25,666 | 4.4 | — | 23,200 | 25,800 | 28,000 | N/A |
| A162 | 0 | — | 4.4 | 0 | 0 | 0 | 0 |
| A163 | 0 | — | 4.4 | 0 | 0 | 0 | 0 |
| A164 | 0 | — | 4.4 | 0 | 0 | 0 | 0 |
| A165 | 5,100 | 3.7 | 0.7 | 4,000 | 6,200 | N/A | N/A |
| A166 | 150 | 2.2 | 2.2 | 120 | 120 | 120 | 240 |
| A167 | 30 | 1.5 | 2.9 | 40 | 80 | 0 | 0 |
| A168 | 0 | — | 4.4 | 0 | 0 | 0 | 0 |
| A169 | 0 | — | 4.4 | 0 | 0 | 0 | 0 |
| A170 | 0 | — | 4.4 | 0 | 0 | 0 | 0 |
| A171 | 0 | — | 4.4 | 0 | 0 | 0 | 0 |
| A172 | 0 | — | 4.4 | 0 | 0 | 0 | 0 |
| A173 | 0 | — | 4.4 | 0 | 0 | 0 | 0 |
| A174 | 0 | — | 4.4 | 0 | 0 | 0 | 0 |
| A175 | 0 | — | 4.4 | 0 | 0 | 0 | 0 |
| A176 | 0 | — | 4.4 | 0 | 0 | 0 | 0 |
| control | 20000 | 4.3 | — | 20200 | 19800 | N/A | N/A |

N/A = replicate not performed

### EXAMPLE 22

Following the protocol of Example 9, the effects of blends of Alimet®, lactic acid, propionic acid (99%, obtained from Sigma Chemical Co., St. Louis, Mo., lot P-1386), and/or butyric acid were studied. Blend formulations are set forth in Table 36. Up to five replicates were performed, and results are reported in Tables 37 and 38.

TABLE 36
acid formulations (g/kg)

| Blend | A¹ | L² | P³ | B⁴ |
|---|---|---|---|---|
| A177 | 0 | 0 | 0 | 10 |
| A178 | 2 | 0 | 0 | 8 |
| A179 | 7.5 | 0 | 0 | 2.5 |
| A180 | 0 | 3.3 | 0 | 6.7 |
| A181 | 2 | 2.67 | 0 | 5.3 |
| A182 | 7.5 | 0.8 | 0 | 1.67 |
| A183 | 0 | 0 | 3.3 | 6.7 |
| A184 | 2 | 0 | 2.67 | 5.3 |
| A185 | 7.5 | 0 | 0.8 | 1.67 |
| A186 | 0 | 6.7 | 0 | 3.3 |
| A187 | 2 | 5.3 | 0 | 2.67 |
| A188 | 7.5 | 1.67 | 0 | 0.8 |
| A189 | 0 | 3.3 | 3.3 | 3.3 |
| A190 | 2 | 2.67 | 2.67 | 2.67 |
| A191 | 7.5 | 0.8 | 0.8 | 0.8 |
| A192 | 0 | 0 | 6.7 | 3.3 |
| A193 | 2 | 0 | 5.3 | 2.67 |
| A194 | 7.5 | 0 | 1.67 | 0.8 |
| A195 | 0 | 10 | 0 | 0 |
| A196 | 2 | 8 | 0 | 0 |
| A197 | 7.5 | 2.5 | 0 | 0 |
| A198 | 0 | 6.7 | 3.3 | 0 |
| A199 | 2 | 5.3 | 2.67 | 0 |
| A200 | 7.5 | 1.67 | 0.8 | 0 |
| A201 | 0 | 3.3 | 6.7 | 0 |
| A202 | 2 | 2.67 | 5.3 | 0 |
| A203 | 7.5 | 0.8 | 1.67 | 0 |
| A204 | 0 | 0 | 10 | 0 |
| A205 | 2 | 0 | 8 | 0 |
| A206 | 7.5 | 0 | 2.5 | 0 |
| A207 | 10 | 0 | 0 | 0 |

¹Alimet ®
²Lactic acid
³Propionic acid
⁴Butyric acid

TABLE 37

| Trial: | Average | | | 1 | 2 | 3 |
|---|---|---|---|---|---|---|
| Blend | cfu/g | log | Δ log | cfu/g | cfu/g | cfu/g |
| control | 20,067 | 4.3 | | 17,200 | 18,800 | 24,200 |
| A177 | 6,333 | 3.8 | 0.5 | 5,400 | 7,000 | 6,600 |
| A178 | 500 | 2.7 | 1.6 | 400 | 600 | N/A |
| A179 | 400 | 2.6 | 1.7 | 200 | 600 | N/A |
| A180 | 1,400 | 3.1 | 1.2 | 1,400 | 1,400 | N/A |
| A181 | 1,500 | 3.2 | 1.1 | 2,000 | 1,000 | N/A |
| A182 | 100 | 2.0 | 2.3 | 200 | 0 | N/A |
| A183 | 6,000 | 3.8 | 0.5 | 9,600 | 2,400 | N/A |
| A184 | 6,900 | 3.8 | 0.5 | 8,800 | 5,000 | N/A |
| A185 | 1,300 | 3.1 | 1.2 | 1,400 | 1,200 | N/A |
| A186 | 4,100 | 3.6 | 0.7 | 5,400 | 2,800 | N/A |
| A187 | 2,400 | 3.4 | 0.9 | 600 | 4,200 | N/A |
| A188 | 400 | 2.6 | 1.7 | 200 | 600 | N/A |
| A189 | 4,700 | 3.7 | 0.6 | 1,000 | 8,400 | N/A |
| A190 | 7,300 | 3.9 | 0.4 | 7,800 | 6,800 | N/A |
| A191 | 300 | 2.5 | 1.8 | 600 | 0 | N/A |
| control | 18,733 | 4.3 | | 20,000 | 22,800 | 13400 |

N/A = replicate not performed

TABLE 38

| Trial: | Average | | | 1 | 2 | 3 | 4 |
|---|---|---|---|---|---|---|---|
| Blend | cfu/g | log | Δ log | cfu/g | cfu/g | cfu/g | cfu/g |
| control | 7,800 | 3.9 | — | 9,800 | 7,200 | 6,400 | N/A |
| A192 | 6,100 | 3.8 | 0.4 | 6,600 | 5,600 | N/A | N/A |
| A193 | 2,600 | 3.4 | 0.8 | 2,200 | 3,000 | N/A | N/A |
| A194 | 25 | 1.4 | 2.8 | 50 | 50 | 0 | 0 |
| A195 | 400 | 2.6 | 1.6 | 800 | 0 | N/A | N/A |
| A196 | 300 | 2.5 | 1.7 | 200 | 400 | N/A | N/A |
| A197 | 0 | — | 4.2 | 0 | 0 | 0 | 0 |
| A198 | 2,400 | 3.4 | 0.8 | 1,000 | 3,800 | N/A | N/A |
| A199 | 600 | 2.8 | 1.4 | 0 | 1,200 | N/A | N/A |
| A200 | 0 | — | 4.2 | 0 | 0 | 0 | 0 |
| A201 | 3,800 | 3.6 | 0.6 | 3,200 | 4,400 | N/A | N/A |
| A202 | 900 | 3.0 | 1.2 | 1,000 | 800 | N/A | N/A |
| A203 | 0 | — | 4.2 | 0 | 0 | 0 | 0 |
| A204 | 3,500 | 3.5 | 0.6 | 5,400 | 1,600 | N/A | N/A |
| A205 | 100 | 2.0 | 2.2 | 200 | 0 | N/A | N/A |
| A206 | 0 | — | 4.2 | 0 | 0 | 0 | 0 |
| A207 | 0 | — | 4.2 | 0 | 0 | 0 | 0 |
| control | 30,600 | 4.5 | — | 41,600 | 19,400 | 30,800 | N/A |

N/A = replicate not performed

### EXAMPLE 23

The ability of Alimet® and DLM to function as palatants for dog and cat food was studied. Alimet® and DLM were added into premium-type dog and cat food in the mixer to test the acceptance of the food compared to food lacking either supplement. The food used comprised good quality protein, and was high in CP and fat. A premium palantant was also added to the food. Alimet® or DLM was added into the mixer prior to extrusion. The formulation of the feline diet is described in Table 39, and of the canine diet in Table 40. The Alimet®/DLM supplementation levels, and intake ratios, are described in Table 41. The intake ratio describes the relative incidence of selecting one food over another.

For the canine study, twenty-one dogs were used (seven small, seven medium, and seven large dogs). For the feline study, twenty mature cats were used. The animals were given two choices of food, placed in separate bowls. Over a two-day period, the dogs were given access to the food for 30 minutes; cats had access for 22 hours. The food chosen and consumed first was observed.

TABLE 39
Feline Diet

| Ingredient | % of total diet |
|---|---|
| Corn | 14.7 |
| Poultry byproduct (low ash) | 20 |
| Soybean Meal | 12.5 |
| Corn gluten meal | 12.2 |
| Meat and Bone meal | 4 |
| Animal fat | 11.4 |
| Rice brewer's | 19.4 |
| Flavor | 2 |
| Fish meal | 2 |
| Dried eggs | 0.5 |
| Salt | 0.5 |
| KCl | 0.5 |
| Vitamins | 0.2 |
| Choline | 0.1 |
| Taurine | 0.1 |
| Tocopherol | 0.05 |
| Trace Minerals | 0.05 |

TABLE 40
Canine Diet

| Ingredient | % of total diet |
|---|---|
| Corn | 33.0 |
| Barley | 15.0 |
| Poultry byproduct (low ash) | 13.0 |
| Poultry byproduct | 12.0 |
| Soybean Meal | 9.0 |
| Animal fat | 8.0 |
| Rice brewer's | 5.0 |
| Flavor | 2.0 |
| Dried eggs | 1.0 |
| Salt | 0.5 |
| Limestone | 0.36 |
| Vitamins | 0.2 |
| Choline | 0.1 |
| Tocopherol | 0.05 |
| Trace Minerals | 0.05 |

TABLE 41

| Foods compared | Intake Ratio |
|---|---|
| Canine | |
| 0.05% Alimet vs. control | 2.07:1 |
| 0.10% Alimet vs. control | 5.58:1 |
| 0.15% Alimet control | 5.13:1 |
| 0.10% DLM vs. control | 5.32:1 |
| 0.15% DLM vs. control | 4.95:1 |
| 0.10% Alimet vs. 0.05% Alimet | 2.54:1 |
| 0.15% Alimet vs. 0.05% Alimet | 1.99:1 |
| 0.15% Alimet vs. 0.10% Alimet | 2.54:1 |
| 0.15% DLM vs. 0.10% DLM | 1.57:1 |
| 0.10% DLM vs. 0.10% Alimet | 1.05:1 |
| 0.15% DLM vs. 0.15% Alimet | 2.5:1 |
| Feline | |
| 0.20% Alimet ® vs. control | 1.67:1 |
| 0.25% Alimet ® vs. control | 1.91:1 |
| 0.30% Alimet ® vs. control | 1.85:1 |
| 0.25% DLM vs. control | 1.87:1 |
| 0.30% DLM vs. control | 1.63:1 |
| 0.25% Alimet ® vs. 0.20% Alimet ® | 1.30:1 |
| 0.30% Alimet ® vs. 0.20% Alimet ® | 1.26:1 |
| 0.30% Alimet ® vs. 0.25% Alimet ® | 1.16:1 |
| 0.30% DLM vs. 0.25% DLM | 1.04:1 |
| 0.25% DLM vs. 0.25% Alimet ® | 1.24:1 |
| 0.30% DLM vs. 0.30% Alimet ® | 1.47:1 |

### EXAMPLE 24

Acceptance of food containing Alimet® or DLM was studied to evaluate dietary consumption under no-choice conditions. Food was offered to the animals (18 dogs: 6 small, 6 medium, 6 large; 15 cats) for one week. The urine pH of six of the cats was also monitored.

The diets described in Tables 39 and 40 above were used. Diets were supplemented with Alimet® or DLM (0.1% for the canine study, 0.25% for the feline study). Additional urine pH tests were carried out with 0.3% Alimet®.

Results of the urine pH experiments are given in Tables 42 and 43. Results of the acceptance text are given in Table 44.

TABLE 42

| | Urine pH | | |
|---|---|---|---|
| Cat No. | Control | Alimet 0.25% | DL Met 0.25% |
| 452 | 6.35 | 6.45 | 6.39 |
| 453 | 6.44 | 6.2 | 6.38 |
| 457 | 6.29 | 6.56 | 6.39 |
| 460 | 6.25 | 6.03 | 6.36 |
| 465 | 6.22 | 6 | 6.1 |
| 475 | 6.56 | 6.44 | 6.36 |

TABLE 43

Urine pH

| Cat No. | Control | Alimet ® 0.3% |
|---|---|---|
| 450 | 6.20 | 6.03 |
| 453 | 6.44 | 6.10 |
| 465 | 6.04 | 5.53 |
| 468 | 6.40 | 5.73 |
| 469 | 6.68 | 6.31 |

TABLE 44

| | |
|---|---|
| Supplementation | Total grams consumed |

| | | |
|---|---|---|
| level | control | supplemented food |

| Canine | | | |
|---|---|---|---|
| Alimet ® | 0.05% | 5,046 | 10,420 |
| | 0.1% | 2,101 | 11,714 |
| | 0.15% | 2,419 | 12,978 |
| DLM | 0.1% | 2,114 | 11,244 |
| | 0.15% | 2,734 | 13,542 |
| Feline | | | |
| Alimet ® | 0.2% | 1,111 | 1,853 |
| | 0.25% | 955 | 1,827 |
| | 0.3% | 1,003 | 1,858 |
| DLM | 0.25% | 985 | 1,842 |
| | 0.3% | 1,078 | 1,754 |

In view of the above, it will be seen that the several objects of the invention are achieved and other advantageous results attained.

As various changes could be made in the above feed rations and methods without departing from the scope of the invention, it is intended that all matter contained in the above description and shown in the accompanying drawing shall be interpreted as illustrative and not in a limiting sense.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

Unless otherwise specified, amounts expressed as percentages are in percent by weight.

What is claimed is:

1. A composition comprising a compound of Formula I and one or more other organic acids, wherein said compound of Formula I has the following structure: 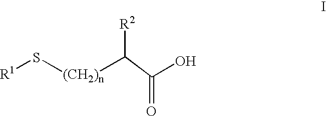
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient.

2. The composition of claim 1 wherein said organic acid derivative is derived from an organic acid having at least one carboxyl moiety and having a pKₐ of less than about 5.5.

3. The composition of claim 2 wherein said organic acid derivative is derived from an organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or mixtures thereof.

4. The composition of claim 3 wherein said other organic acid is an organic acid having at least one carboxyl moiety and having a pKₐ of less than about 5.5.

5. The composition of claim 4 wherein said other organic acid is selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or mixtures thereof.

6. The composition of claim 5 wherein said compound of Formula I is 2-hydroxy-4-(methylthio)butanoic acid, or a salt, ester or amide thereof.

7. The composition of claim 6 wherein said other organic acid is selected from the group consisting of formic acid, acetic acid, propionic acid, butyric acid, lactic acid, citric acid, and fumaric acid.

8. The composition of claim 7 wherein the combined concentration of said compound of Formula I and said other organic acid in said composition is between about 0.1 g/kg and about 50 g/kg.

9. The composition of claim 8 wherein said combined concentration is between about 0.8 g/kg and about 30 g/kg.

10. The composition of claim 8 wherein said combined concentration is between about 1 g/kg and about 25 g/kg.

11. The composition of claim 8 wherein said combined concentration is between about 1 g/kg and about 10 g/kg.

12. The composition of claim 1 comprising 2-hydroxy-4-(methylthio)butanoic acid, or a salt, ester or amide thereof; and
a first organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid.

13. The composition of claim 12 wherein said first organic acid is selected from the group consisting of formic acid, propionic acid, butyric acid, and lactic acid.

14. The composition of claim 12 further comprising an acidulant selected from the group consisting of mineral acids.

15. The composition of claim 14 wherein said acidulant is selected from the group consisting of phosphoric acid, sulfuric acid, phosphorous acid, hydrochloric acid, hydrobromic acid, and nitric acid.

16. The composition of claim 15 further comprising a second organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid.

17. The composition of claim 16 wherein said first organic acid and second organic acid are independently selected from the group consisting of formic acid, propionic acid, butyric acid, and lactic acid.

18. The composition of claim 17 wherein said first organic acid is formic acid, said second organic acid is propionic acid, and said acidulant is phosphoric acid.

19. The composition of claim 18 wherein the content of 2-hydroxy-4-(methylthio)butanoic acid is from about 5% to about 20% of the sum of the 2-hydroxy-4-(methylthio)butanoic acid, formic acid, propionic acid, and phosphoric acid content;
the content of the formic acid is from about 65% to about 85% of said sum;
the content of the propionic acid is from about 1% to about 15% of said sum; and
the content of the phosphoric acid is from about 5% to about 20% of said sum.

20. The composition of claim 19 wherein the content of 2-hydroxy-4-(methylthio)butanoic acid is about 10% of said sum;
the content of the formic acid is about 75% of said sum;
the content of the propionic acid is about 5% of said sum; and
the content of the phosphoric acid is about 10% of said sum.

21. The composition of claim 18 wherein the content of 2-hydroxy-4-(methylthio)butanoic acid is from about 20% to about 40% of the sum of the 2-hydroxy-4-(methylthio)butanoic acid, formic acid, propionic acid, and phosphoric acid content;
the content of the formic acid is from about 45% to about 65% of said sum;
the content of the propionic acid is from about 1% to about 20% of said sum; and
the content of the phosphoric acid is from about 1% to about 15% of said sum.

22. The composition of claim 21 wherein the content of 2-hydroxy-4-(methylthio)butanoic acid is about 30% of said sum;
the content of the formic acid is about 55% of said sum;
the content of the propionic acid is about 10% of said sum; and
the content of the phosphoric acid is about 5% of said sum.

23. The composition of claim 17 wherein said first organic acid is butyric acid, said second organic acid is lactic acid, and said acidulant is phosphoric acid.

24. The composition of claim 23 wherein the content of 2-hydroxy-4-(methylthio)butanoic acid is from about 20% to about 40% of the sum of the 2-hydroxy-4-(methylthio)butanoic acid, butyric acid, lactic acid, and phosphoric acid content;
the content of the butyric acid is from about 10% to about 30% of said sum;
the content of the lactic acid is from about 10% to about 30% of said sum; and
the content of the phosphoric acid is from about 20% to about 40% of said sum.

25. The composition of claim 24 wherein the content of 2-hydroxy-4-(methylthio)butanoic acid is about 30% of said sum;
the content of the butyric acid is about 20% of said sum;
the content of the lactic acid is about 20% of said sum; and
the content of the phosphoric acid is about 30% of said sum.

26. The composition of claim 23 wherein the content of 2-hydroxy-4-(methylthio)butanoic acid is from about 20% to about 40% of the sum of the 2-hydroxy-4-(methylthio)butanoic acid, butyric acid, lactic acid, and phosphoric acid content;
the content of the butyric acid is from about 5% to about 25% of said sum;
the content of the lactic acid is from about 10% to about 30% of said sum; and
the content of the phosphoric acid is from about 25% to about 45% of said sum.

27. The composition of claim 26 wherein the content of 2-hydroxy-4-(methylthio)butanoic acid is about 30% of said sum;
the content of the butyric acid is about 15% of said sum;
the content of the lactic acid is about 20% of said sum; and
the content of the phosphoric acid is about 35% of said sum.

28. The composition of claim 16 further comprising a third organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid.

29. The composition of claim 28 wherein said first organic acid is butyric acid, said second organic acid is formic acid, said third organic acid is lactic acid, and said acidulant is phosphoric acid.

30. The composition of claim 29 wherein the content of 2-hydroxy-4-(methylthio)butanoic acid is from about 10% to about 30% of the sum of the 2-hydroxy-4-(methylthio)butanoic acid, butyric acid, formic acid, lactic acid, and phosphoric acid content;
the content of the butyric acid is from about 2% to about 22% of said sum;
the content of the formic acid is from about 20% to about 40% of said sum;
the content of the lactic acid is from about 8% to about 28% of said sum; and
the content of the phosphoric acid is from about 10% to about 30% of said sum.

31. The composition of claim 30 wherein the content of 2-hydroxy-4-(methylthio)butanoic acid is about 20% of said sum;
the content of the butyric acid is about 12% of said sum;
the content of the formic acid is about 30% of said sum;
the content of the lactic acid is about 18% of said sum; and
the content of the phosphoric acid is about 20% of said sum.

32. The composition of claim 28 wherein said first organic acid is butyric acid, said second organic acid is lactic acid, said third organic acid is propionic acid, and said acidulant is phosphoric acid.

33. The composition of claim 32 wherein the content of 2-hydroxy-4-(methylthio)butanoic acid is from about 10% to about 30% of the sum of the 2-hydroxy-4-(methylthio)butanoic acid, butyric acid, lactic acid, propionic acid, and phosphoric acid content;
the content of the butyric acid is from about 2% to about 22% of said sum;
the content of the lactic acid is from about 8% to about 28% of said sum;
the content of the propionic acid is from about 20% to about 40% of said sum; and
the content of the phosphoric acid is from about 10% to about 30% of said sum.

34. The composition of claim 33 wherein the content of 2-hydroxy-4-(methylthio)butanoic acid is about 20% of said sum;
the content of the butyric acid is about 12% of said sum;
the content of the lactic acid is about 18% of said sum;
the content of the propionic acid is about 30% of said sum; and
the content of the phosphoric acid is about 20% of said sum.

35. The composition of claim 28 wherein said first organic acid is butyric acid, said second organic acid is formic acid, said third organic acid is propionic acid, and said acidulant is phosphoric acid.

36. The composition of claim 35 wherein the content of 2-hydroxy-4-(methylthio)butanoic acid is from about 1% to about 20% of the sum of the 2-hydroxy-4-(methylthio)butanoic acid, butyric acid, formic acid, propionic acid, and phosphoric acid content;
the content of the butyric acid is from about 1% to about 15% of said sum;
the content of the formic acid is from about 65% to about 85% of said sum;
the content of the propionic acid is from about 1% to about 15% of said sum; and
the content of the phosphoric acid is from about 1% to about 15% of said sum.

37. The composition of claim 36 wherein the content of 2-hydroxy-4-(methylthio)butanoic acid is about 10% of said sum;
the content of the butyric acid is about 5% of said sum;
the content of the formic acid is about 75% of said sum;
the content of the propionic acid is about 5% of said sum; and
the content of the phosphoric acid is about 5% of said sum.

38. The composition of claim 12 further comprising a second organic acid, wherein said first organic acid and said second organic acid are independently selected from the group consisting of formic acid, propionic acid, butyric acid, and lactic acid.

39. The composition of claim 38 wherein said first organic acid is formic acid and said second organic acid is propionic acid.

40. The composition of claim 39 wherein the content of 2-hydroxy-4-(methylthio)butanoic acid is from about 20% to about 40% of the sum of the 2-hydroxy-4-(methylthio)butanoic acid, formic acid, and propionic acid content;
the content of the formic acid is from about 40% to about 60% of said sum; and
the content of the propionic acid is from about 10% to about 30% of said sum.

41. The composition of claim 40 wherein the content of 2-hydroxy-4-(methylthio)butanoic acid is about 30% of said sum;
the content of the formic acid is about 50% of said sum; and
the content of the propionic acid is about 20% of said sum.

42. A composition comprising a compound of Formula I and an acidulant, wherein said compound of Formula I has the following structure: 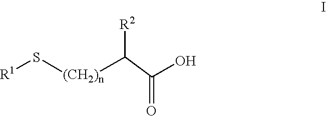
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient;
wherein said acidulant is selected from the group consisting of mineral acids.

43. The composition of claim 42 wherein said organic acid derivative is derived from an organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or mixtures thereof.

44. The composition of claim 43 wherein said acidulant is selected from the group consisting of phosphoric acid, sulfuric acid, phosphorous acid, hydrochloric acid, hydrobromic acid, and nitric acid.

45. The composition of claim 44 wherein the content of the 2-hydroxy-4-(methylthio)butanoic acid is from about 5% to about 50% of the sum of the 2-hydroxy-4-(methylthio)butanoic acid and acidulant content.

46. The composition of claim 45 wherein the content of the 2-hydroxy-4-(methylthio)butanoic acid is about 5% of said sum.

47. The composition of claim 45 wherein the content of the 2-hydroxy-4-(methylthio)butanoic acid is about 25% of said sum.

48. The composition of claim 45 wherein the content of the 2-hydroxy-4-(methylthio)butanoic acid is about 45% of said sum.

49. A combination comprising a compound of Formula I and one or more other organic acids, wherein said compound of Formula I has the following structure: 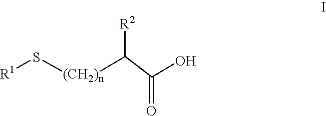
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient.

50. The combination of claim 49 wherein said organic acid derivative is derived from an organic acid having at least one carboxyl moiety and having a pKₐ of less than about 5.5.

51. The combination of claim 50 wherein said organic acid derivative is derived from an organic acid selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or combinations thereof.

52. The combination of claim 51 wherein said other organic acid is an organic acid having at least one carboxyl moiety and having a pKₐ of less than about 5.5.

53. The combination of claim 52 wherein said other organic acid is selected from the group consisting of formic acid, acetic acid, propionic acid, butyric acid, benzoic acid, lactic acid, malic acid, tartaric acid, mandelic acid, citric acid, fumaric acid, sorbic acid, boric acid, succinic acid, adipic acid, glycolic acid, and glutaric acid, or combinations thereof.

54. The combination of claim 53 wherein said compound of Formula I is 2-hydroxy-4-(methylthio)butanoic acid, or a salt, ester or amide thereof.

55. The combination of claim 49 comprising
2-hydroxy-4-(methylthio)butanoic acid, or a salt, ester or amide thereof; and
a first organic acid selected from the group consisting of formic acid, propionic acid, butyric acid, and lactic acid.

56. The combination of claim 55 further comprising an acidulant selected from the group consisting of phosphoric acid, sulfuric acid, phosphorous acid, hydrochloric acid, hydrobromic acid, and nitric acid.

57. The combination of claim 56 further comprising a second organic acid selected from the group consisting of formic acid, propionic acid, butyric acid, and lactic acid.

58. The combination of claim 57 wherein said first organic acid is formic acid, said second organic acid is propionic acid, and said acidulant is phosphoric acid.

59. The combination of claim 57 wherein said first organic acid is butyric acid, said second organic acid is lactic acid, and said acidulant is phosphoric acid.

60. The combination of claim 57 further comprising a third organic acid selected from the group consisting of formic acid, propionic acid, butyric acid, and lactic acid.

61. The combination of claim 60 wherein said first organic acid is butyric acid, said second organic acid is formic acid, said third organic acid is lactic acid, and said acidulant is phosphoric acid.

62. The combination of claim 60 wherein said first organic acid is butyric acid, said second organic acid is lactic acid, said third organic acid is propionic acid, and said acidulant is phosphoric acid.

63. The combination of claim 60 wherein said first organic acid is butyric acid, said second organic acid is formic acid, said third organic acid is propionic acid, and said acidulant is phosphoric acid.

64. The combination of claim 55 further comprising a second organic acid selected from the group consisting of formic acid, propionic acid, butyric acid, and lactic acid.

65. The combination of claim 64 wherein said first organic acid is formic acid and said second organic acid is propionic acid.

66. A combination comprising a compound of Formula I and an acidulant, wherein said compound of Formula I has the following structure: 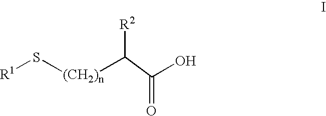
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient;
wherein said acidulant is selected from the group consisting of phosphoric acid, sulfuric acid, phosphorous acid, hydrochloric acid, hydrobromic acid, and nitric acid.

67. The combination of claim 66 wherein said organic acid derivative is derived from an organic acid selected from the group consisting of formic acid, propionic acid, butyric acid, and lactic acid.

68. The combination of claim 67 wherein the content of the compound of Formula I is from about 5% to about 50% of the sum of the compound of Formula I and acidulant content.

69. An animal feed composition comprising a compound of Formula I and one or more other organic acids, wherein said compound of Formula I has the following structure: 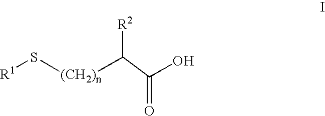
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient.

70. The composition of claim 69 comprising 2-hydroxy-4-(methylthio)butanoic acid, or a salt, ester or amide thereof; and
a first organic acid selected from the group consisting of formic acid, propionic acid, butyric acid, and lactic acid.

71. The composition of claim 70 further comprising an acidulant selected from the group consisting of phosphoric acid, sulfuric acid, phosphorous acid, hydrochloric acid, hydrobromic acid, and nitric acid.

72. The composition of claim 71 further comprising a second organic acid selected from the group consisting of formic acid, propionic acid, butyric acid, and lactic acid.

73. The composition of claim 70 further comprising a second organic acid selected from the group consisting of formic acid, propionic acid, butyric acid, and lactic acid.

74. An animal feed composition comprising a compound of Formula I and an acidulant, wherein said compound of Formula I has the following structure: 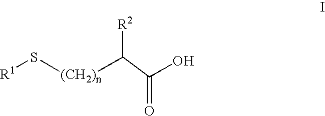
wherein R¹ is an alkyl group having from one to four carbon atoms;
n is an integer from 0 to 2;
R² is selected from the group consisting of hydroxy, amino, —OCOR³, or —NHCOR³; and
R³ is an organic acid derivative;
or a salt thereof;
and an acceptable diluent, adjuvant or excipient;
wherein said acidulant is selected from the group consisting of phosphoric acid, sulfuric acid, phosphorous acid, hydrochloric acid, hydrobromic acid, and nitric acid.

75. A method of inhibiting or killing microbes in a subject, comprising treating said subject with a composition according to claim 1.

76. The method of claim 75 wherein said subject is water or food.

77. The method of claim 76 wherein said food is selected from the group consisting of human food, livestock food, pet food, or aquaculture food.

78. The method of claim 77 wherein said composition is mixed with the food as it is formulated.

79. The method of claim 78 wherein said composition is applied to a pre-mixed or pre-pelleted feed.

80. The method of claim 79 wherein said composition, subsequent to treating said food, is uniformly dispersed throughout said food.

81. The method of claim 76 wherein said food comprises a meat or bone meal.

82. The method of claim 76 wherein said food is dry food.

83. The method of claim 76 wherein said food is liquid food.

84. The method of claim 76 wherein said food is a combination of dry feed and liquid food.

85. The method of claim 76 wherein said food is fed to an animal.

86. The method of claim 85 wherein said animal is a ruminant animal.

87. The method of claim 86 wherein said ruminant animal is selected from the group consisting of dairy cows, lactating dairy cows, dairy calves, beef cattle, sheep, and goats.

88. The method of claim 85 wherein said animal is an aquaculture.

89. The method of claim 88 wherein said aquaculture is fish or crustaceans.

90. The method of claim 85 wherein said animal is livestock.

91. The method of claim 90 wherein said livestock is swine or horses.

92. The method of claim 85 wherein said animal is poultry.

93. The method of claim 92 wherein said poultry is selected from the group consisting of chickens, turkeys, and hatchlings thereof.

94. The method of claim 85 wherein said animal is a companion animal.

95. The method of claim 94 wherein said companion animal is a dog or a cat.

96. The method of claim 75 wherein said microbe is a bacterium.

97. The method of claim 75 wherein said microbe is a mold.

98. A method of killing mold in food or water comprising corn and soy, the method comprising applying to said food or water a composition according to claim 1.

99. A method of killing mold in food having a moisture content of from 0-17%, the method comprising applying to said food a composition according to claim 1.

100. The method of claim 99 wherein said moisture content is at least 0.01% by weight of the food.

101. The method of claim 99 wherein said moisture content is at least 1% by weight of the food.

102. The method of claim 99 wherein said moisture content is at least 5% by weight of the food.

103. The method of claim 99 wherein said moisture content is at least 10% by weight of the food.

104. The method of claim 75 wherein said animal food is heat-treated, either before or after application of said composition.

105. A method for enhancing the palatability of animal food, the method comprising treating the food with a compound of Formula I: 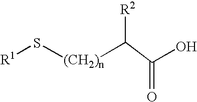
wherein R¹ is an alkyl group having from one to four carbon atoms;
n is an integer from 0 to 2;
R² is selected from the group consisting of hydroxy, amino, —OCOR³, or —NHCOR³;
and wherein R³ is an organic acid derivative;
or a salt thereof.
wherein the concentration of the compound of Formula I in the food is between about 0.01 wt. % and about 0.5 wt. %.

106. The method of claim 105 wherein said compound of Formula I is 2-hydroxy-4-(methylthio)butanoic acid or d,1-methionine, or a salt, ester or amide thereof.

107. The method of claim 106 wherein said concentration is between about 0.05 wt. % and about 0.3 wt. %.

108. The method of claim 107 wherein said food is canine, feline, or aquaculture food.

109. The method of claim 108 wherein said food is canine food and said concentration is about 0.10 wt. %.

110. The method of claim 108 wherein said food is feline food and said concentration is about 0.25 wt. %.

111. The composition of claim 72 further comprising a third organic acid selected from the group consisting of formic acid, propionic acid, butyric acid, and lactic acid.

112. The composition of claim 73 further comprising a second organic acid selected from the group consisting of formic acid, propionic acid, butyric acid, and lactic acid.
